(19) United States
(12) Patent Application Publication
(10) Pub. No.: US 2023/0063748 A1
(43) Pub. Date: Mar. 2, 2023

(54) IMIDAZOPYRIDINE-BASED ARYLAMINE COMPOUND AND USE THEREOF

(71) Applicant: GUANGDONG AGLAIA OPTOELECTRONIC MATERIALS CO., LTD, Foshan, Guangdong (CN)

(72) Inventors: Shaofu CHEN, Foshan (CN); Liangliang YAN, Foshan (CN); Lei DAI, Foshan (CN); Lifei CAI, Foshan (CN)

(73) Assignee: GUANGDONG AGLAIA OPTOELECTRONIC MATERIALS CO., LTD, Foshan, Guangdong (CN)

(21) Appl. No.: 17/777,027

(22) PCT Filed: Sep. 19, 2020
(86) PCT No.: PCT/CN2020/116333

§ 371 Date: May 14, 2022

(30) Foreign Application Priority Data
Dec. 11, 2019 (CN) 201911268938.0

(51) Int. Cl. C07D 403/14; C07D 405/14; C07D 409/14; C07D 519/00; C07D 471/04

(52) U.S. Cl. CPC C07D 403/14; C07D 405/14; C07D 409/14; C07D 519/00; C07D 471/04

(57) ABSTRACT

The present invention relates to an imidazopyridine-based arylamine compound and an application thereof. The compound has a structure as shown in Formula I. The compound of the present invention has the advantages such as, a low sublimation temperature, a good thermal stability, a high refractive index, and a small refractive index difference in the visible light region, and can be used as a light extraction layer material for use in an organic light-emitting device.

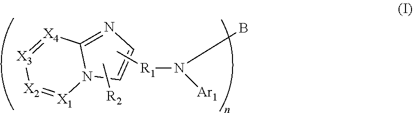

### TECHNICAL FIELD

The present invention relates to the technical field of organic light-emitting materials, and in particular to an imidazopyridine-based arylamine compound and an application thereof in organic light-emitting devices.

### BACKGROUND

At present, the organic light-emitting device (OLED), as a new generation of display technology, has achieved more and more attention in the aspect of display and lighting technology and has wide application prospect. But, compared with the demands for market application, the luminous efficiency, driving voltage, service life and other properties of the OLED device still need to be strengthened and improved.

Generally, the basic structure of OLED device is to sandwich various functions of organic functional material films between metal electrodes, just like a sandwiched structure. Driven by electric current, holes and electrons are injected from cathode and anode, and then compounded on a light-emitting layer after moving a certain distance, then released in a form of light or heat, thus producing the light emission of OLED. However, organic functional materials are core components of the OLED. The material heat stability, photochemical stability, electrochemical stability, quantum yield, film-forming stability, crystallinity, color saturation and the like are the major indicators to influence the performances of the device.

On the one hand, how to narrow the huge gap between internal and external quantum efficiency of an OLED, and how to reduce the total emission effect in device and to improve the optical coupling extraction ratio have aroused people's extensive attention. Materials of the current light extraction layer have a relatively low refractive index, especially in red light wave band; and usually, the refractive index is less than 1.85, a few are greater than 1.90, and the fewer are greater than 2.0. In addition, the current light extraction materials have larger differences of refractive index in the regions of red, green and blue light wave bands, resulting in larger differences in the optimum thickness of the three colors of light, which is thus incapable of fully reflecting the properties of light extraction materials. For top emitters, the larger the refractive index of the light extraction layer material is, the higher the corresponding external quantum efficiency is, and the higher the device luminous efficiency is. Therefore, it is rather important to develop a light extraction layer material with high refractive index. CN103828485 and TW201506128 have disclosed a light extraction layer material with polybiphenyl diamine as a core, but the refractive index is still lightly low, especially, the refractive index needs to be further improved in the aspect of red light.

### SUMMARY

Directed to the shortcomings in the field above, the present invention provides an imidazopyridine-based arylamine compound; such kind of compound has the advantages such as, a low sublimation temperature, a good thermal stability, a high refractive index, and a small refractive index difference in the visible light region and thus, can be applied to an organic light-emitting device.

An imidazopyridine-based arylamine compound, has a structural formula as shown in Formula I:

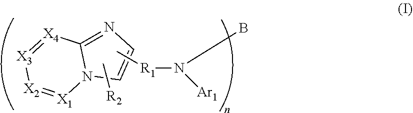

where n is 1 or 2;

X1, X2, X3 and X4 independently represent CR₀ or N; R₀ is independently selected from hydrogen, deuterium, halogen, C1-C8 alkyl, C1-C8 heteroalkyl, aralkyl, amino, silicyl, substituted or unsubstituted C6-C60 aryl, substituted or unsubstituted C1-C60 heteroaryl, nitrile, and isonitrile; and adjacent R₀ are capable of being bonded to form a fused ring;

R₁ is a single bond, C1-C30 alkylene, C1-C30 heteroalkylene, C3-C30 cycloalkylene, substituted or unsubstituted C6-C30 arylene, and substituted or unsubstituted C2-C28 heteroarylene;

R₂ is independently selected from hydrogen, deuterium, halogen, C1-C30 alkyl, C1-C30 heteroalkyl, C3-C30 cycloalkyl, C1-C30 alkoxy, C6-C60 aryloxy, amino, silicyl, nitrile, isonitrile, phosphino, substituted or unsubstituted C6-C60 aryl, and substituted or unsubstituted C1-C60 heteroaryl;

Ar₁ is substituted or unsubstituted C6-C60 aryl, substituted or unsubstituted C6-C60 heteroaryl, C3-C60 cycloalkyl, and substituted or unsubstituted C6-C60 arylamino;

B is substituted or unsubstituted C6-C60 aryl or arylene, substituted or unsubstituted C6-C60 heteroaryl or heteroarylene, substituted or unsubstituted C3-C60 cycloalkyl or cycloalkylene, and substituted or unsubstituted C6-C60 arylamino or arylimino;

and where one or more carbon atoms in heteroalkyl or heteroaryl are replaced by at least one heteroatom selected from O, S, N, Se, Si and Ge; the substitution refers to a substitution by deuterium, halogen, C1-C30 alkyl, phenyl, naphthyl or biphenyl.

Preferably, the structural formula is as shown in Formula II below:

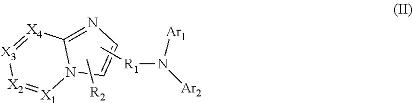

where R₁ is a single bond, C1-C10 alkylene, C1-C10 heteroalkylene, C3-C10 cycloalkylene, substituted or unsubstituted C6-C30 arylene, and substituted or unsubstituted C2-C28 heteroarylene;

R₂ is independently selected from hydrogen, deuterium, halogen, C1-C10 alkyl, C1-C10 heteroalkyl, C1-C10 alkoxy, C3-C30 cycloalkyl, C6-C30 aryloxy, amino, silicyl, nitrile, isonitrile, phosphino, substituted or unsubstituted C6-C30 aryl, and substituted or unsubstituted C1-C30 heteroaryl;

Ar₁ and Ar₂ are substituted or unsubstituted C6-C30 aryl, substituted or unsubstituted C6-C30 heteroaryl, substituted or unsubstituted monocyclic or polycyclic C3-C30 aliphatic ring or aromatic ring, and substituted or unsubstituted C6-C30 arylamino;

and where one or more carbon atoms in heteroalkyl or heteroaryl are replaced by at least one heteroatom selected from O, S, N, and Si; the substitution refers to a substitution by deuterium, halogen, C1-C8 alkyl, phenyl, naphthyl or biphenyl.

Preferably, R₁ is C1-C8 alkyl substituted or unsubstituted C6-C20 arylene, C1-C8 alkyl substituted or unsubstituted C2-C18 heteroarylene; R₂ is C1-C8 alkyl, C1-C8 alkyl substituted or unsubstituted C6-C20 aryl, C1-C8 alkyl substituted or unsubstituted C1-C20 heteroaryl; Ar₁ and Ar₂ are substituted or unsubstituted C6-C20 aryl, substituted or unsubstituted C6-C20 heteroaryl, substituted or unsubstituted monocyclic or polycyclic C3-C20 aliphatic ring or aromatic ring, substituted or unsubstituted C6-C20 arylamino; where one or more carbon atoms in heteroalkyl or heteroaryl are replaced by at least one heteroatom selected from O, S and N; the substitution refers to a substitution by deuterium, C1-C8 alkyl, phenyl, naphthyl or biphenyl.

More preferably, R₁ is C1-C4 substituted or unsubstituted C6-C10 arylene, C1-C4 alkyl substituted or unsubstituted C2-C8 heteroarylene; Ar₁ and R₂ are C1-C4 alkyl substituted or unsubstituted C6-C10 aryl, C1-C4 alkyl substituted or unsubstituted C1-C8 heteroaryl; wherein Ar₂ is substituted or unsubstituted C6-C20 aryl, substituted or unsubstituted C6-C20 heteroaryl, substituted or unsubstituted monocyclic or polycyclic C3-C20 aliphatic ring or aromatic ring, substituted or unsubstituted C6-C20 arylamino; where one or more carbon atoms in heteroalkyl or heteroaryl are replaced by at least one heteroatom selected from O, S and N; the substitution refers to a substitution by deuterium, C1-C4 alkyl, phenyl, naphthyl or biphenyl.

As a preferred compound, at least one of Ar₁ or Ar₂ contains the following structural formula (III), where R₁ is a single bond, C1-C8 alkylene, C1-C8 heteroalkylene, C3-C8 cycloalkylene, C1-C8 alkyl substituted or unsubstituted C6-C30 arylene, C1-C8 alkyl substituted or unsubstituted C2-C28 heteroarylene; where R₂ is hydrogen, deuterium, halogen, C1-C8 alkyl, C1-C8 heteroalkyl, C3-C8 cycloalkyl, C1-C8 alkyl substituted or unsubstituted C6-C30 aryl, and C1-C8 alkyl substituted or unsubstituted C1-C30 heteroaryl.

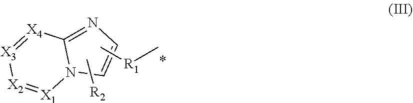

A preferred compound may be a structure as shown in Formula (IV) below, where R₁ is a single bond, C1-C8 alkylene, C1-C8 heteroalkylene, C3-C8 cycloalkylene, C1-C8 alkyl substituted or unsubstituted C6-C30 arylene, C1-C8 alkyl substituted or unsubstituted C2-C28 heteroarylene; wherein R₂ is hydrogen, deuterium, halogen, C1-C8 alkyl, C1-C8 heteroalkyl, C3-C8 cycloalkyl, C1-C8 alkyl substituted or unsubstituted C6-C30 aryl, and C1-C8 alkyl substituted or unsubstituted C1-C30 heteroaryl; and where Ar₁ is substituted or unsubstituted C6-C30 aryl, substituted or unsubstituted C6-C30 heteroaryl, substituted or unsubstituted monocyclic or polycyclic C3-C30 aliphatic ring or aromatic ring, and A is substituted or unsubstituted C6-C30 arylene, and substituted or unsubstituted C6-C30 heteroarylene; and where one or more carbon atoms in heteroalkyl or heteroaryl are replaced by at least one heteroatom selected from O, S, N and Si; and the substitution refers to a substitution by deuterium, halogen, C1-C8 alkyl, phenyl, naphthyl or biphenyl.

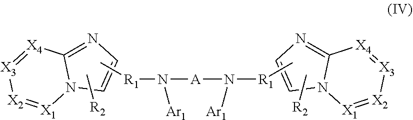

Preferably, R₁ is C1-C8 alkyl substituted or unsubstituted C6-C20 arylene, C1-C8 alkyl substituted or unsubstituted C2-C18 heteroarylene; R₂ is C1-C8 alkyl, C1-C8 alkyl substituted or unsubstituted C6-C20 aryl, C1-C8 alkyl substituted or unsubstituted C1-C20 heteroaryl; Ar₁ is substituted or unsubstituted C6-C20 aryl, substituted or unsubstituted C6-C20 heteroaryl, substituted or unsubstituted monocyclic or polycyclic C3-C20 aliphatic ring or aromatic ring; A is substituted or unsubstituted C6-C20 arylene, and substituted or unsubstituted C6-C20 heteroarylene; where one or more carbon atoms in heteroalkyl or heteroaryl are replaced by at least one heteroatom selected from O, S and N; the substitution refers to a substitution by deuterium, C1-C8 alkyl, phenyl, naphthyl or biphenyl.

Preferably, where X1, X2, X3 and X4 independently represent CR₀; and R₀ is independently selected from hydrogen, and C1-C8 alkyl.

A preferred compound is the following compound:

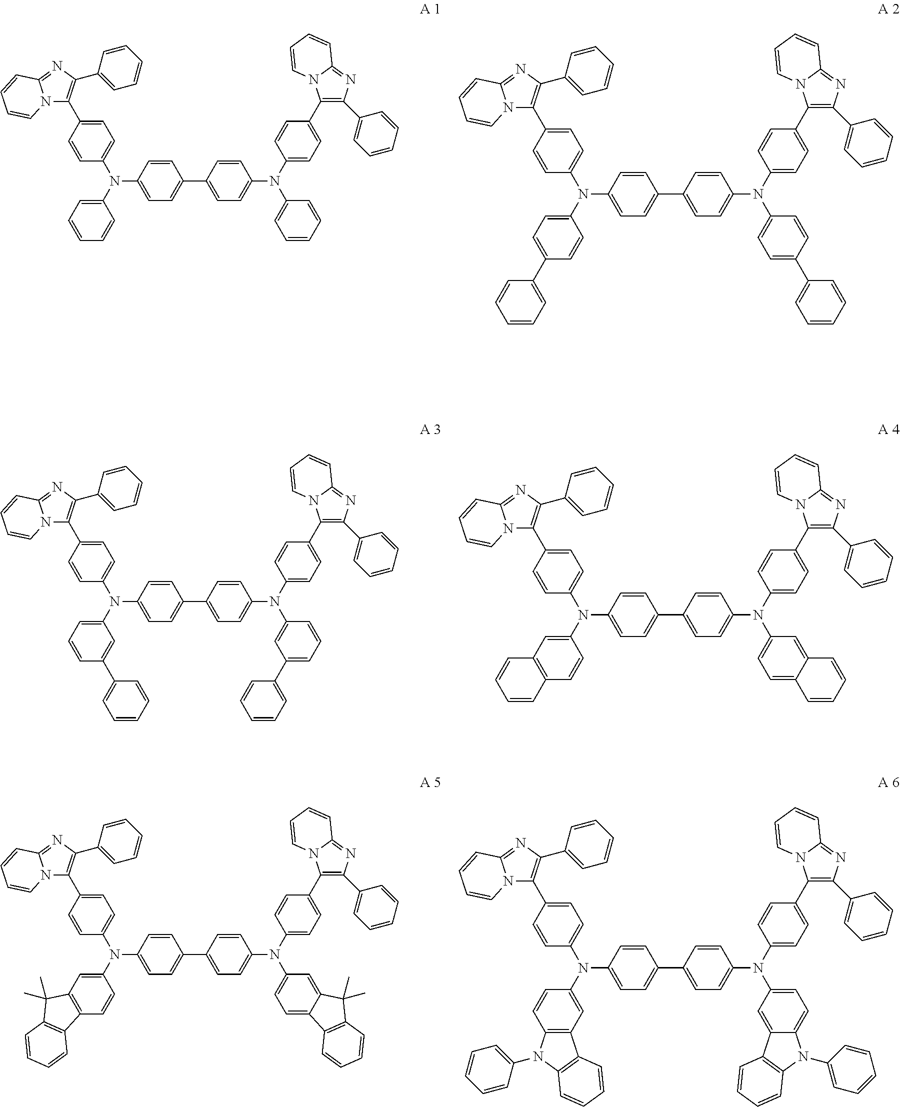

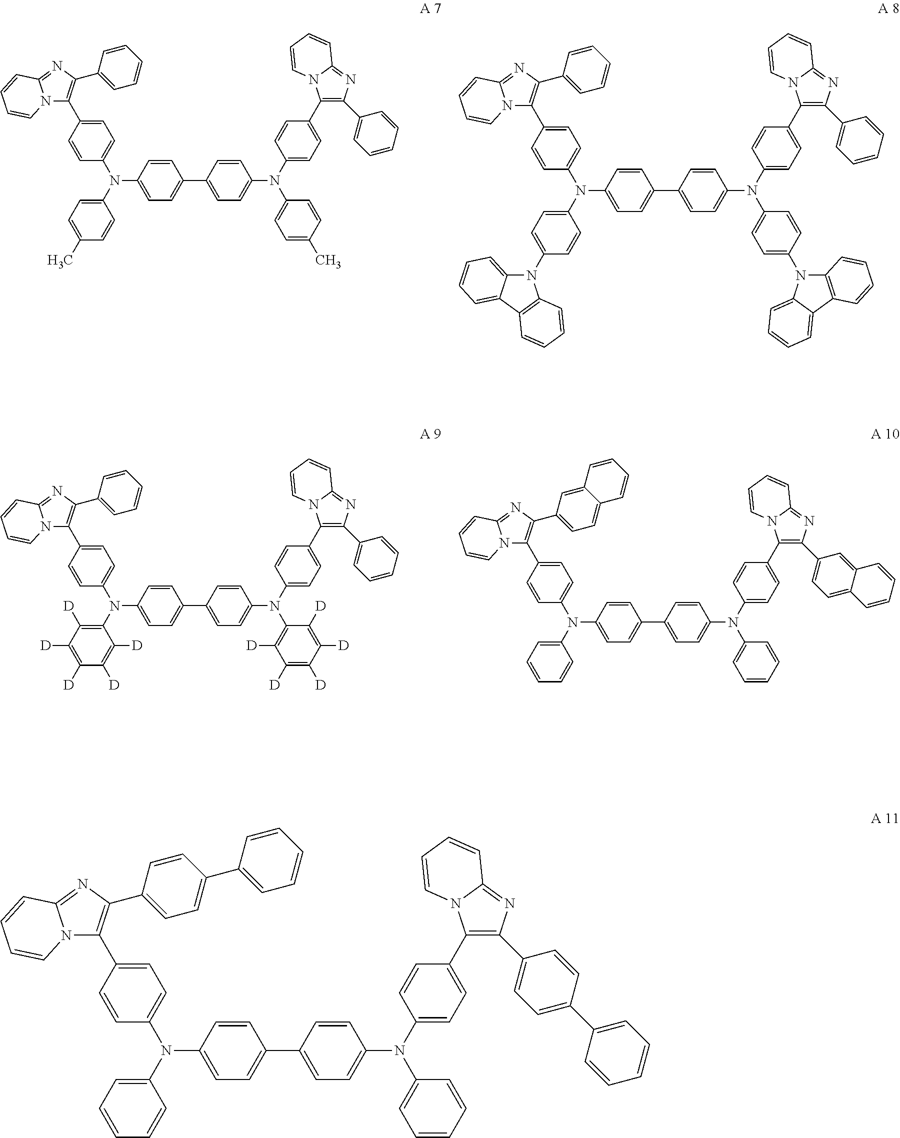

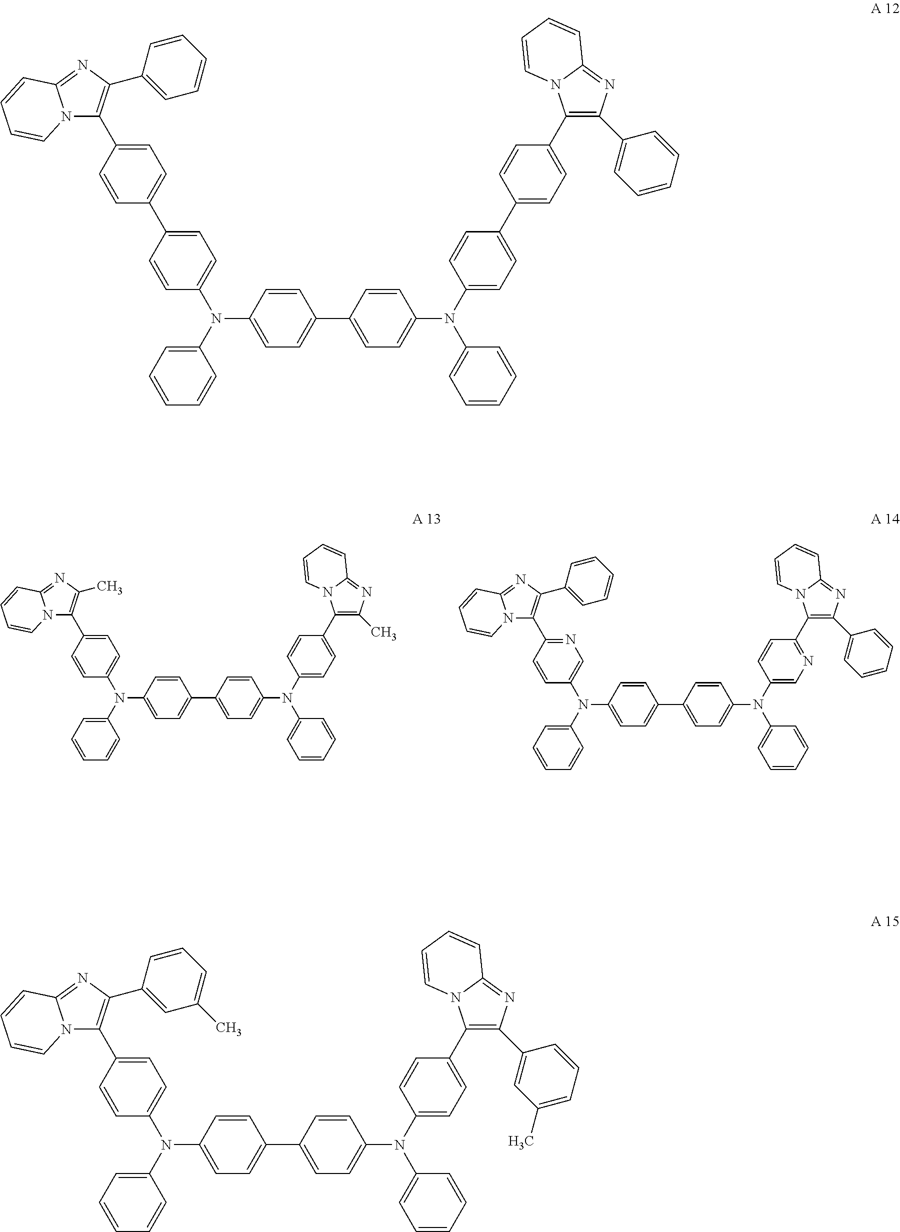 [SMILES: c1ccc(-c2nc3ccccn3c2-c2ccc(-c3ccc(N(c4ccccc4)c4ccc(-c5ccc(N(c6ccccc6)c6ccc(-c7ccc(-c8c(-c9ccccc9)nc9ccccn89)cc7)cc6)cc5)cc4)cc3)cc2)cc1 ; Cc1nc2ccccn2c1-c1ccc(N(c2ccccc2)c2ccc(-c3ccc(N(c4ccccc4)c4ccc(-c5c(C)nc6ccccn56)cc4)cc3)cc2)cc1 ; c1ccc(-c2nc3ccccn3c2-c2ccc(N(c3ccccc3)c3ccc(-c4ccc(N(c5ccccc5)c5ccc(-c6c(-c7ccccc7)nc7ccccn67)nc5)cc4)cc3)cn2)cc1 ; Cc1cccc(-c2nc3ccccn3c2-c2ccc(N(c3ccccc3)c3ccc(-c4ccc(N(c5ccccc5)c5ccc(-c6c(-c7cccc(C)c7)nc7ccccn67)cc5)cc4)cc3)cc2)c1]

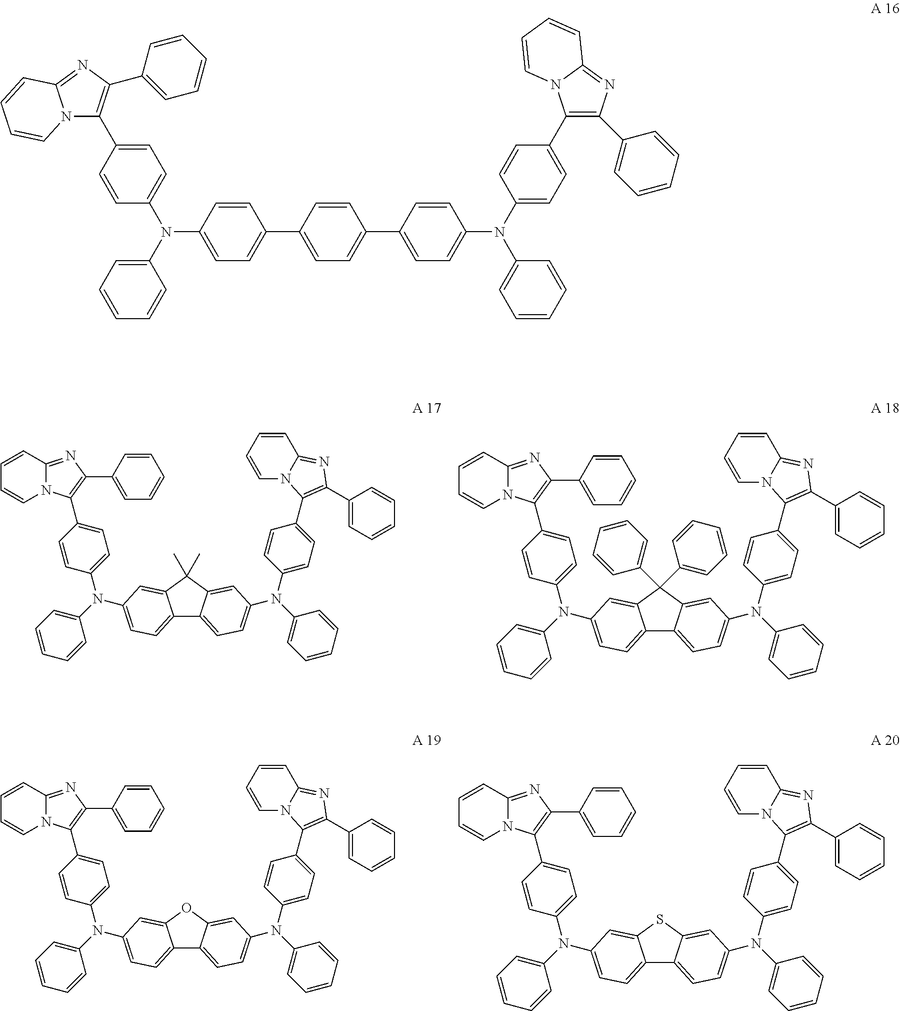

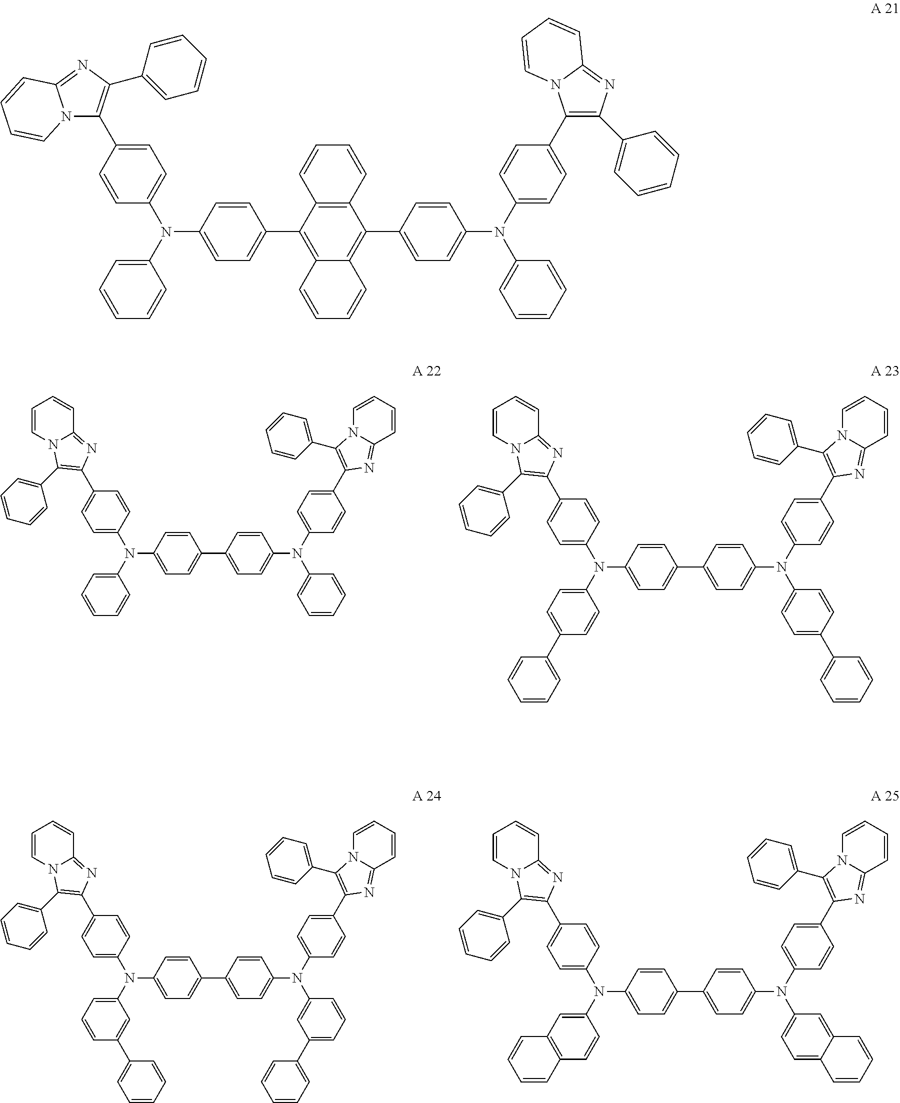

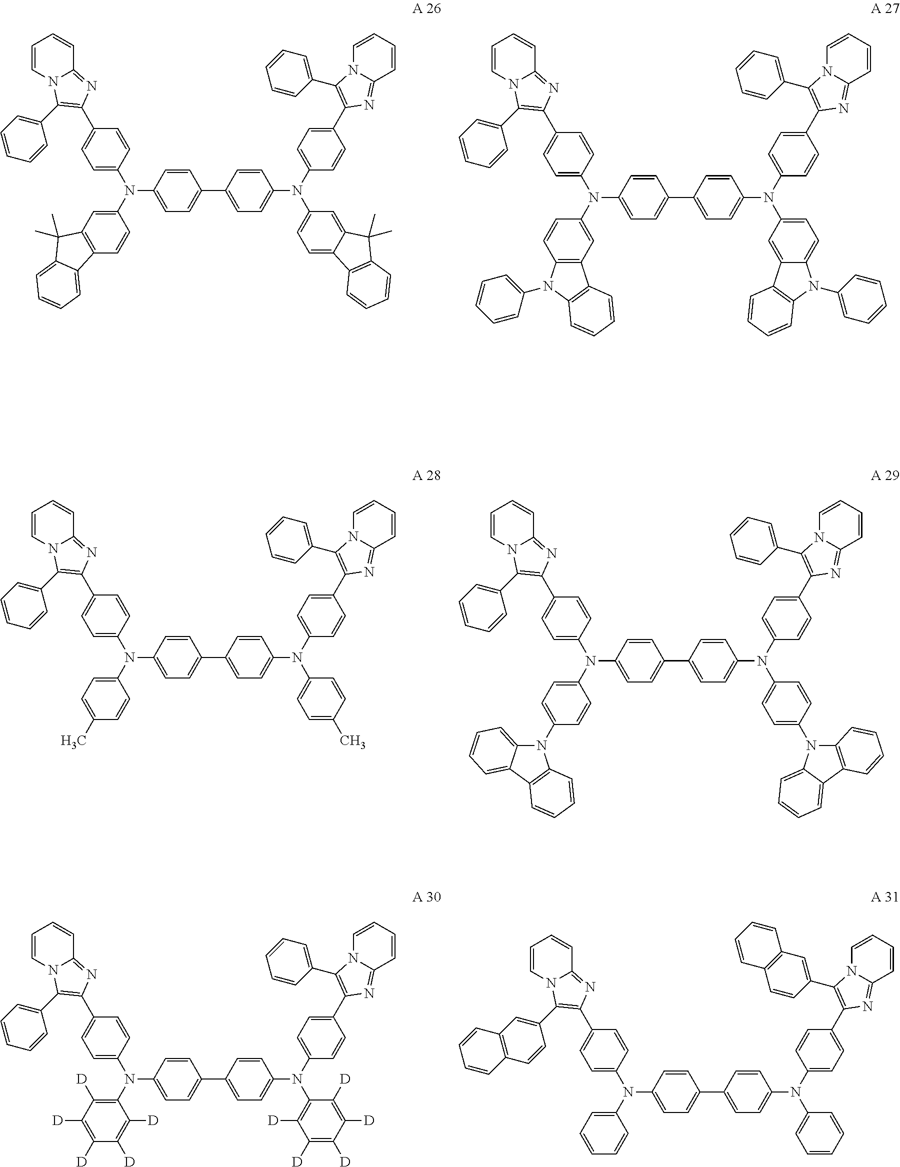

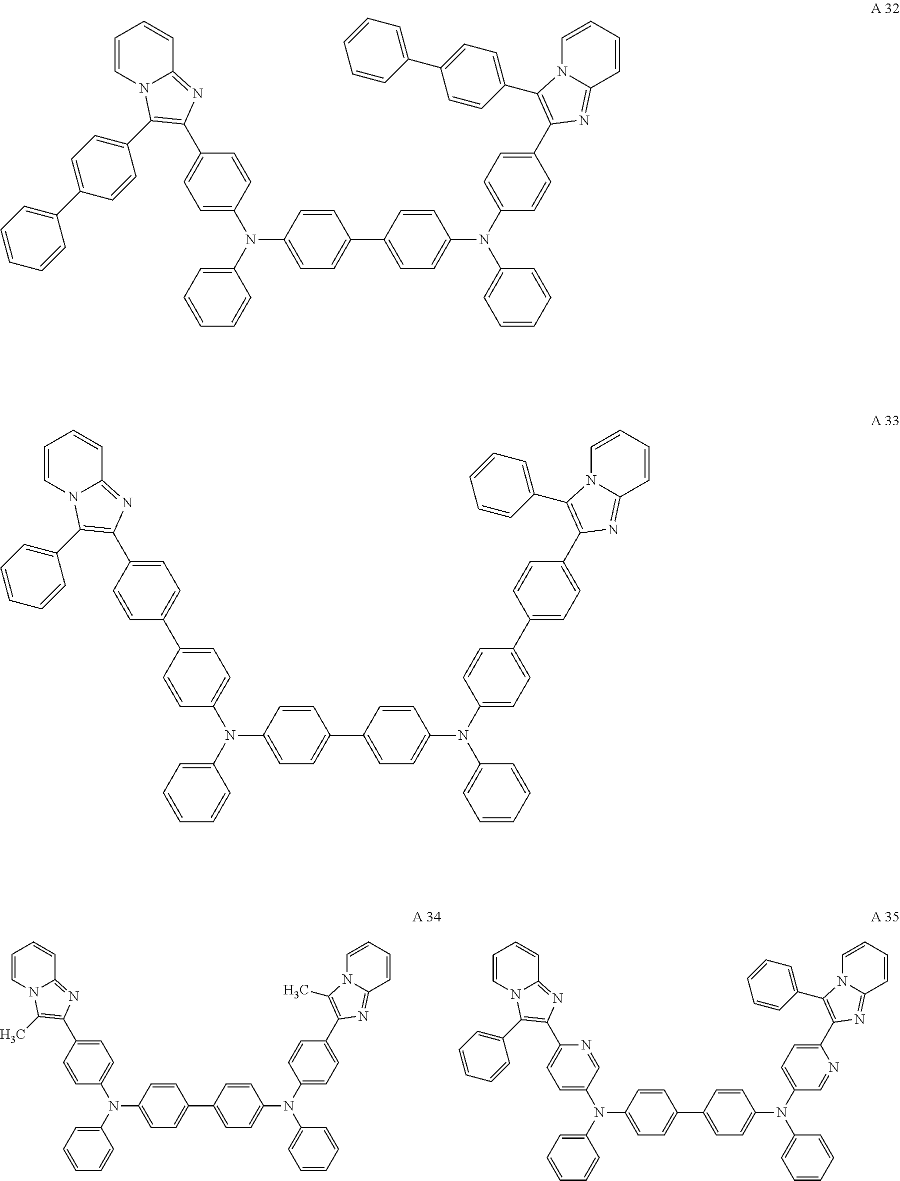 [SMILES: c1ccc(-c2ccc(-c3c(-c4ccc(N(c5ccccc5)c5ccc(-c6ccc(N(c7ccccc7)c7ccc(-c8nc9ccccn9c8-c8ccc(-c9ccccc9)cc8)cc7)cc6)cc5)cc4)nc4ccccn34)cc2)cc1 ; c1ccc(-c2c(-c3ccc(-c4ccc(N(c5ccccc5)c5ccc(-c6ccc(N(c7ccccc7)c7ccc(-c8ccc(-c9nc%10ccccn%10c9-c9ccccc9)cc8)cc7)cc6)cc5)cc4)cc3)nc3ccccn23)cc1 ; Cc1c(-c2ccc(N(c3ccccc3)c3ccc(-c4ccc(N(c5ccccc5)c5ccc(-c6nc7ccccn7c6C)cc5)cc4)cc3)cc2)nc2ccccn12 ; c1ccc(-c2c(-c3ccc(N(c4ccccc4)c4ccc(-c5ccc(N(c6ccccc6)c6ccc(-c7nc8ccccn8c7-c7ccccc7)nc6)cc5)cc4)cn3)nc3ccccn23)cc1]

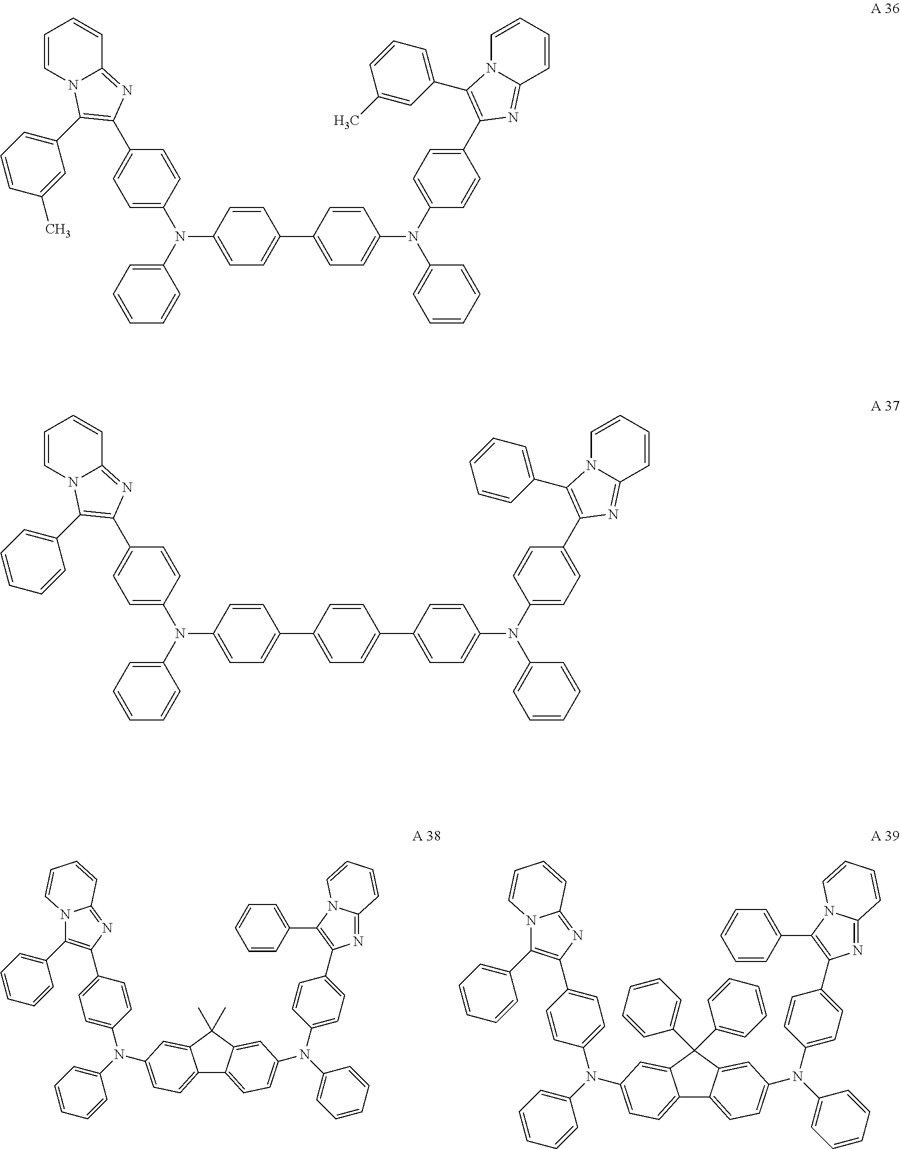

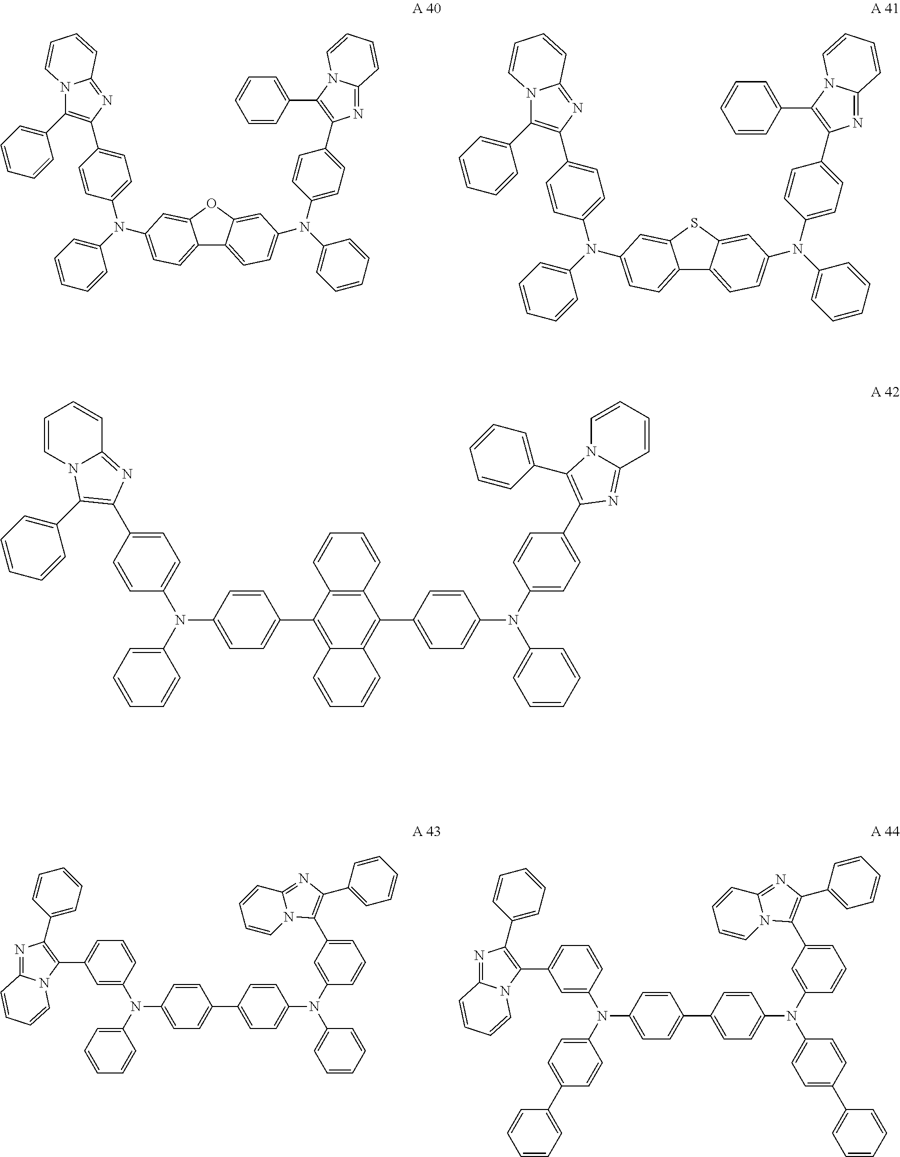

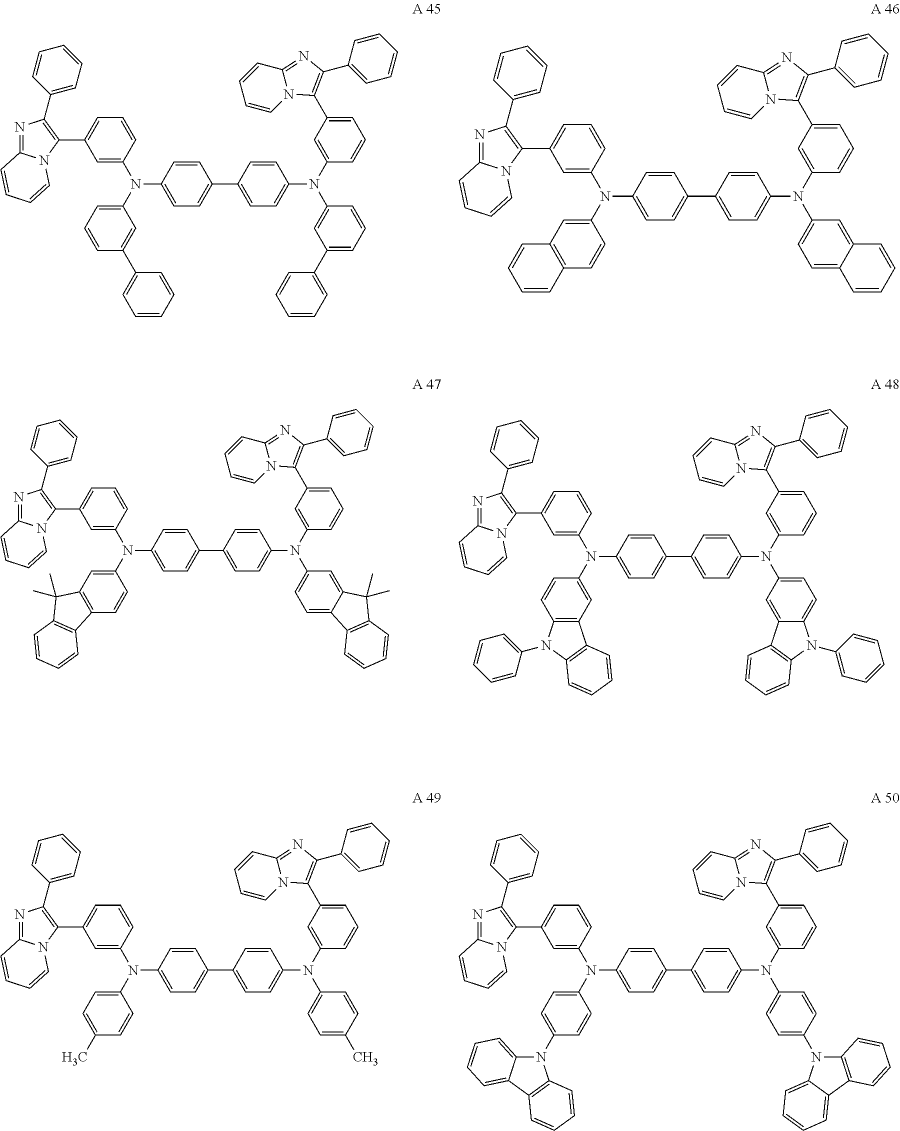

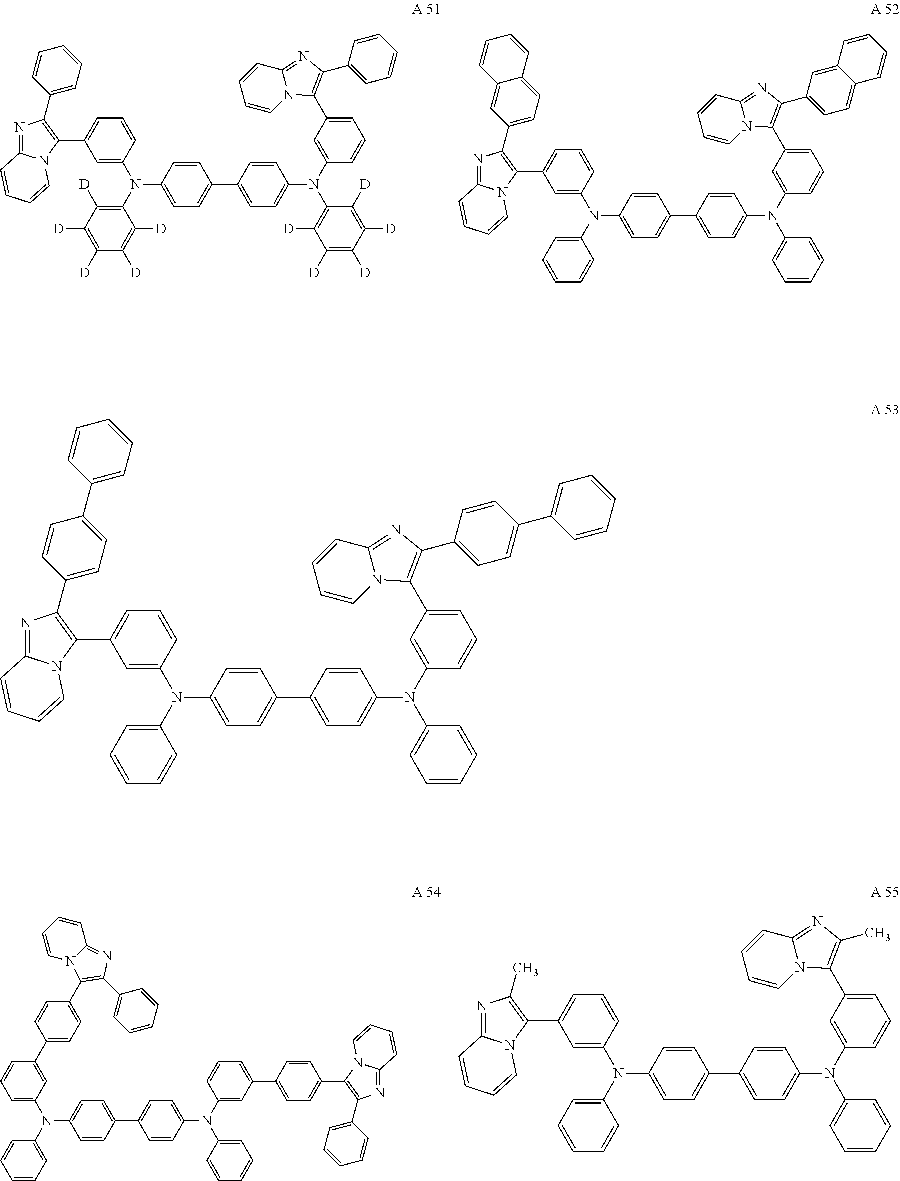

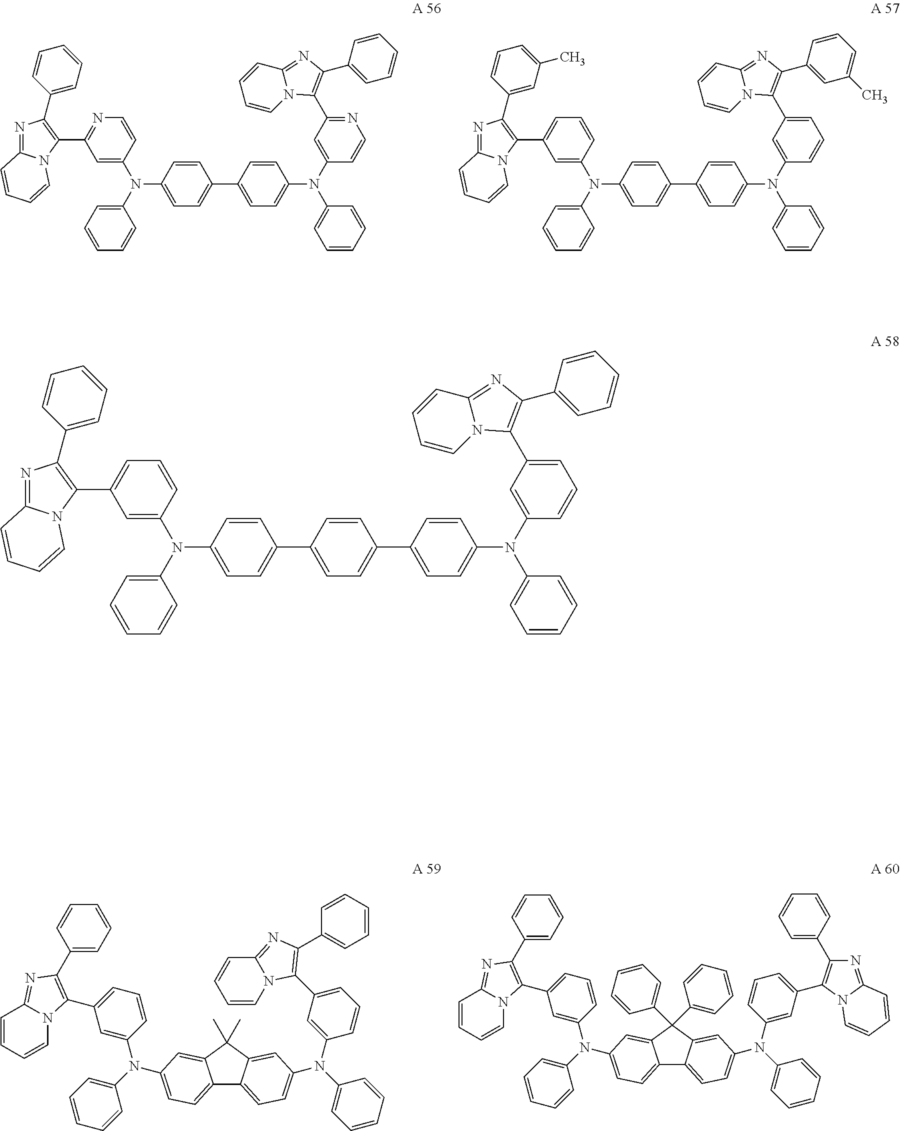

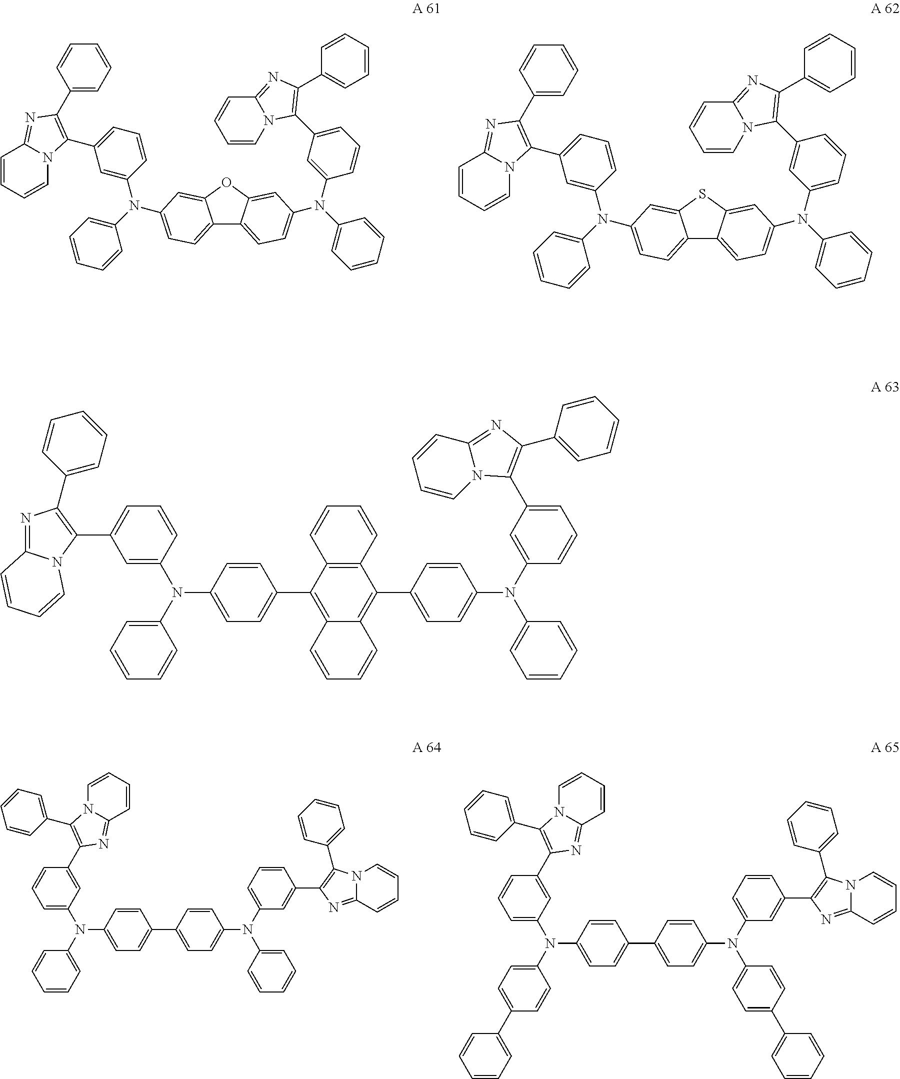

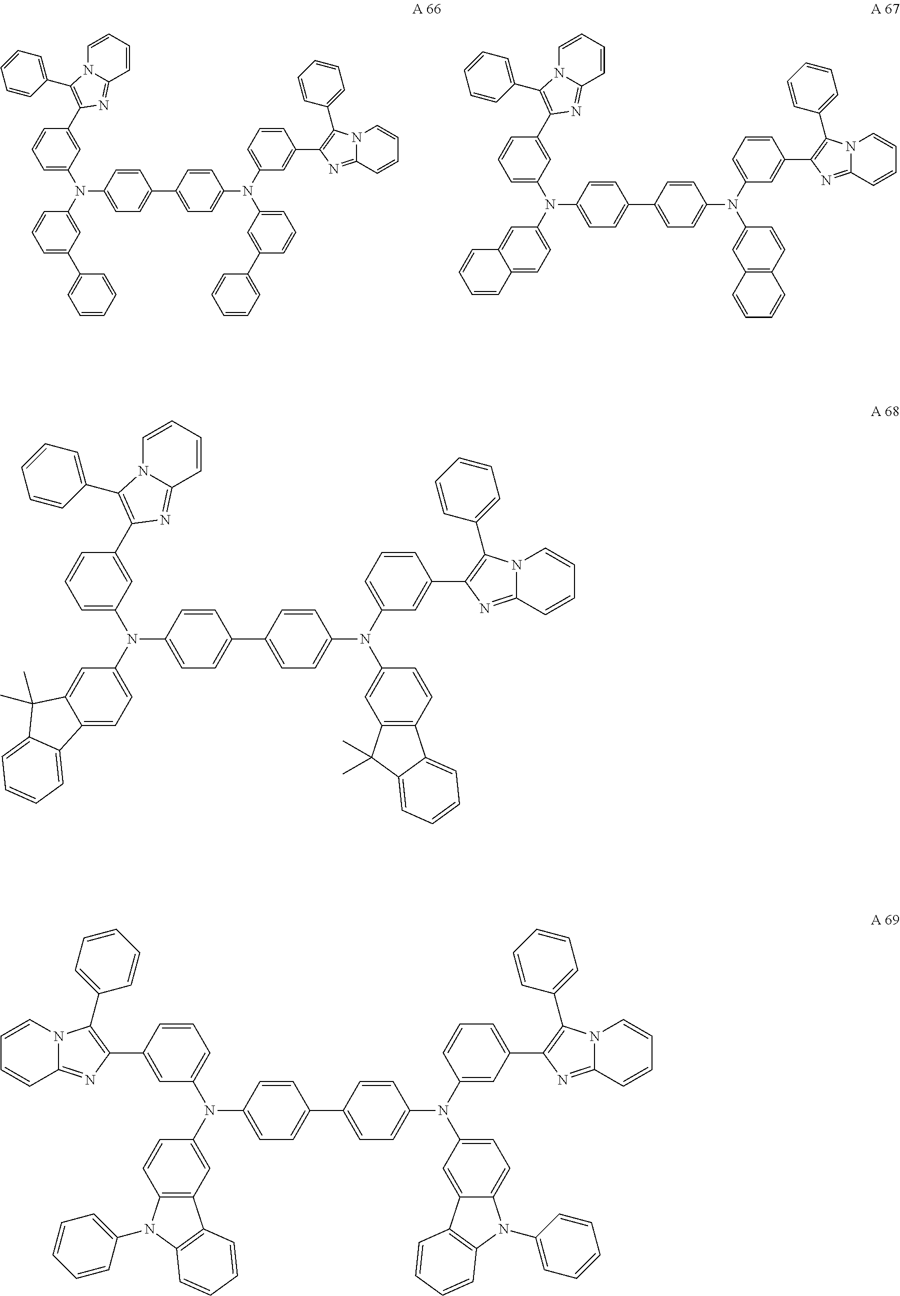

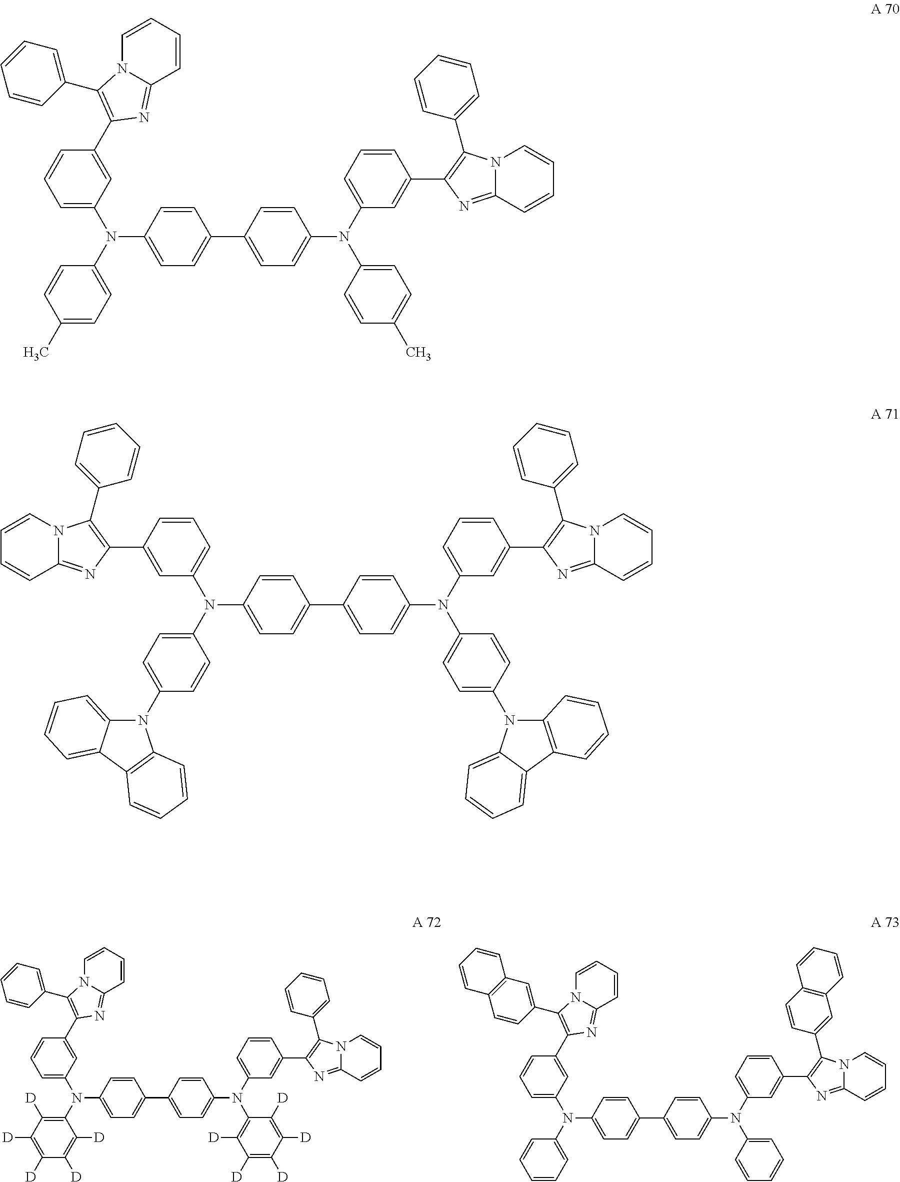

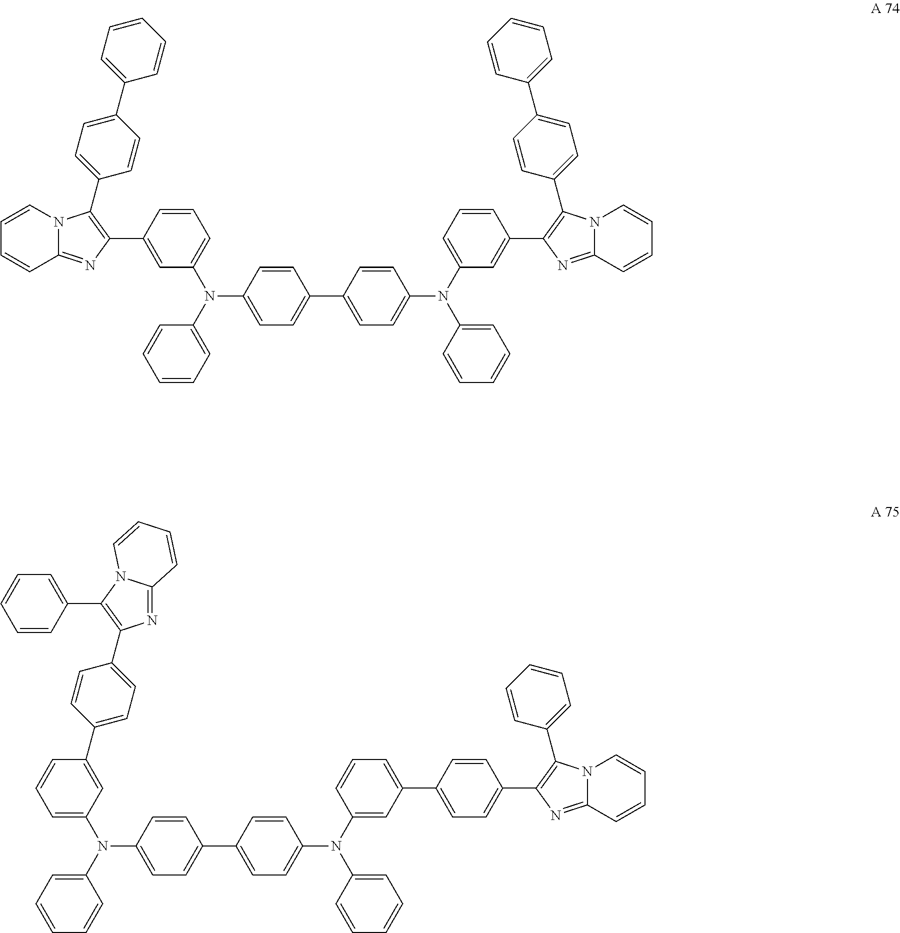 [SMILES: c1ccc(-c2ccc(-c3c(-c4cccc(N(c5ccccc5)c5ccc(-c6ccc(N(c7ccccc7)c7cccc(-c8nc9ccccn9c8-c8ccc(-c9ccccc9)cc8)c7)cc6)cc5)c4)nc4ccccn34)cc2)cc1 ; c1ccc(-c2c(-c3ccc(-c4cccc(N(c5ccccc5)c5ccc(-c6ccc(N(c7ccccc7)c7cccc(-c8ccc(-c9nc%10ccccn%10c9-c9ccccc9)cc8)c7)cc6)cc5)c4)cc3)nc3ccccn23)cc1]

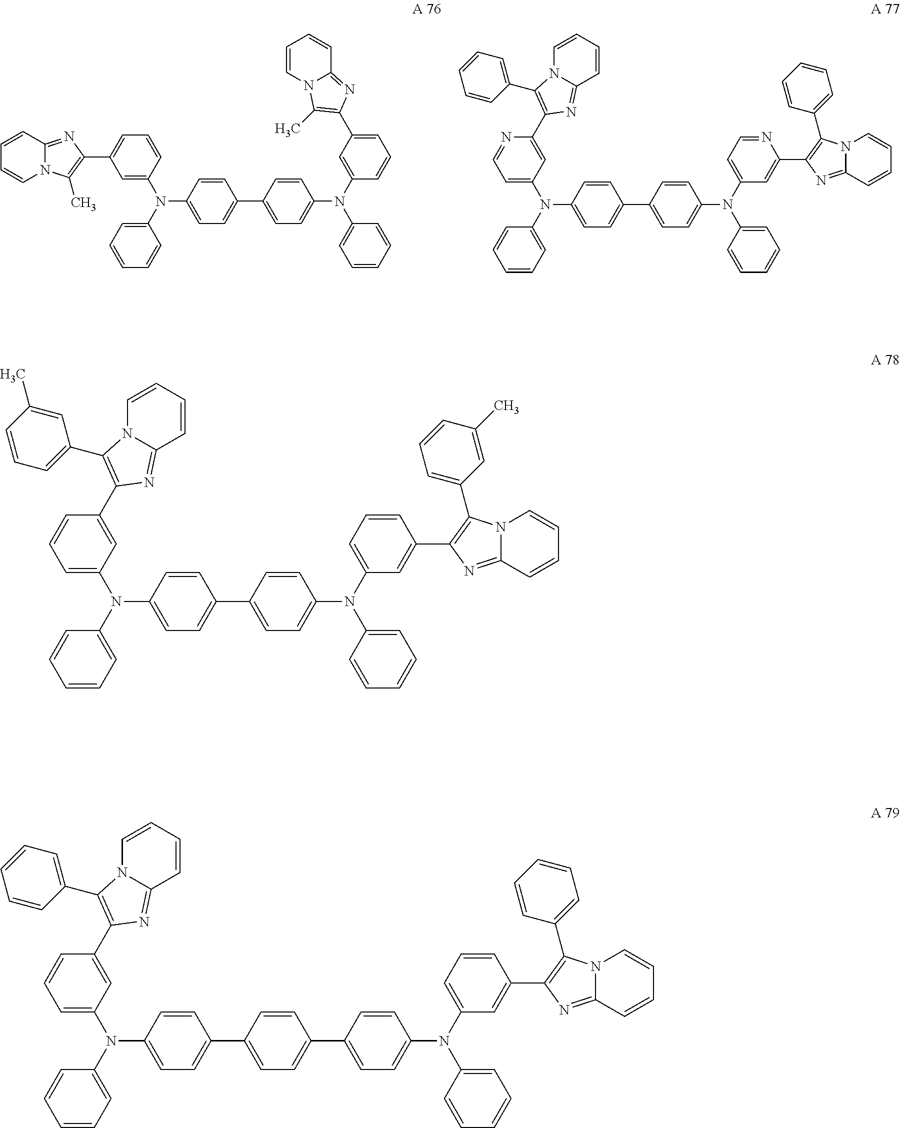 [SMILES: Cc1c(-c2cccc(N(c3ccccc3)c3ccc(-c4ccc(N(c5ccccc5)c5cccc(-c6nc7ccccn7c6C)c5)cc4)cc3)c2)nc2ccccn12 ; c1ccc(-c2c(-c3cc(N(c4ccccc4)c4ccc(-c5ccc(N(c6ccccc6)c6ccnc(-c7nc8ccccn8c7-c7ccccc7)c6)cc5)cc4)ccn3)nc3ccccn23)cc1 ; Cc1cccc(-c2c(-c3cccc(N(c4ccccc4)c4ccc(-c5ccc(N(c6ccccc6)c6cccc(-c7nc8ccccn8c7-c7cccc(C)c7)c6)cc5)cc4)c3)nc3ccccn23)c1 ; c1ccc(-c2c(-c3cccc(N(c4ccccc4)c4ccc(-c5ccc(-c6ccc(N(c7ccccc7)c7cccc(-c8nc9ccccn9c8-c8ccccc8)c7)cc6)cc5)cc4)c3)nc3ccccn23)cc1]

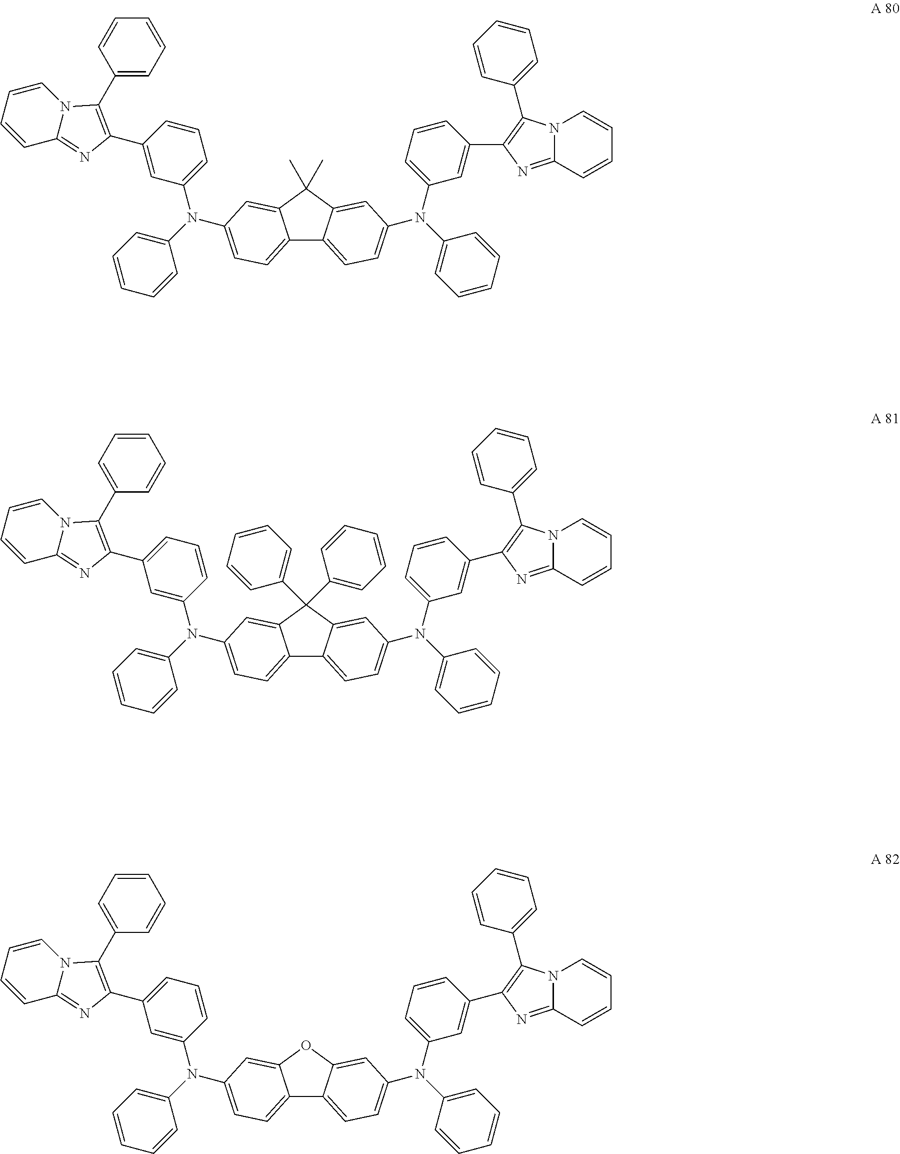 [SMILES: CC1(C)c2cc(N(c3ccccc3)c3cccc(-c4nc5ccccn5c4-c4ccccc4)c3)ccc2-c2ccc(N(c3ccccc3)c3cccc(-c4nc5ccccn5c4-c4ccccc4)c3)cc21 ; c1ccc(-c2c(-c3cccc(N(c4ccccc4)c4ccc5c(c4)C(c4ccccc4)(c4ccccc4)c4cc(N(c6ccccc6)c6cccc(-c7nc8ccccn8c7-c7ccccc7)c6)ccc4-5)c3)nc3ccccn23)cc1 ; c1ccc(-c2c(-c3cccc(N(c4ccccc4)c4ccc5c(c4)oc4cc(N(c6ccccc6)c6cccc(-c7nc8ccccn8c7-c7ccccc7)c6)ccc45)c3)nc3ccccn23)cc1]

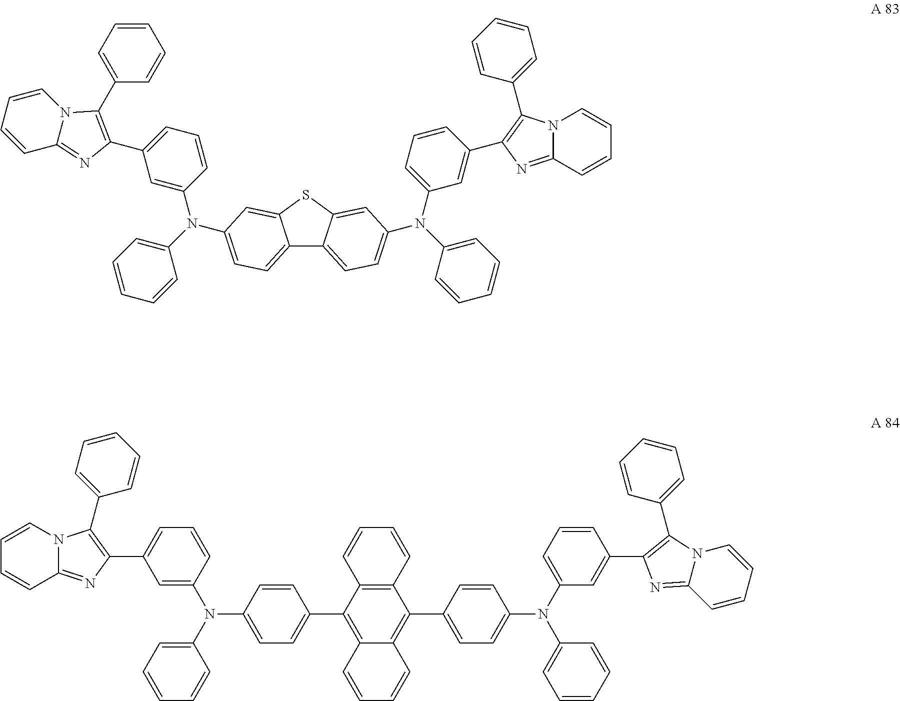 [SMILES: c1ccc(-c2c(-c3cccc(N(c4ccccc4)c4ccc5c(c4)sc4cc(N(c6ccccc6)c6cccc(-c7nc8ccccn8c7-c7ccccc7)c6)ccc45)c3)nc3ccccn23)cc1 ; c1ccc(-c2c(-c3cccc(N(c4ccccc4)c4ccc(-c5c6ccccc6c(-c6ccc(N(c7ccccc7)c7cccc(-c8nc9ccccn9c8-c8ccccc8)c7)cc6)c6ccccc56)cc4)c3)nc3ccccn23)cc1]

A preferred compound is the following compound:

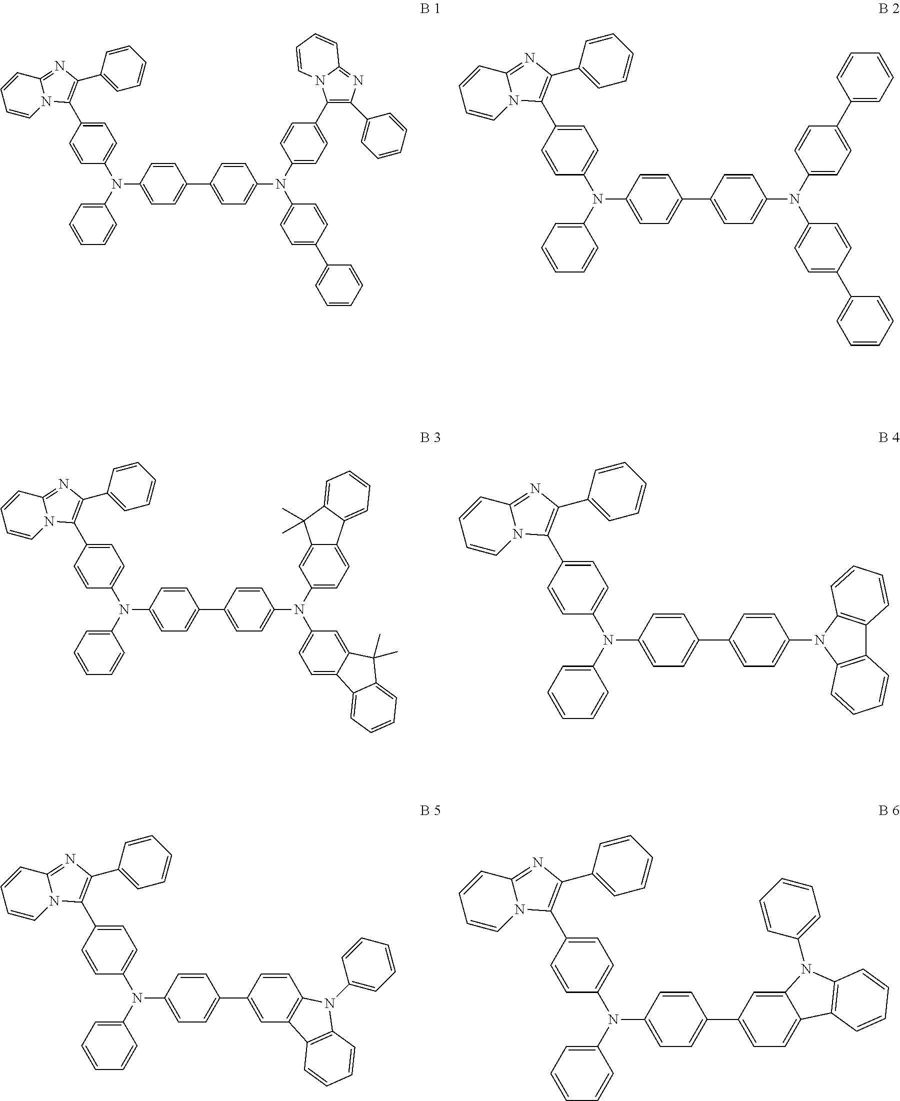

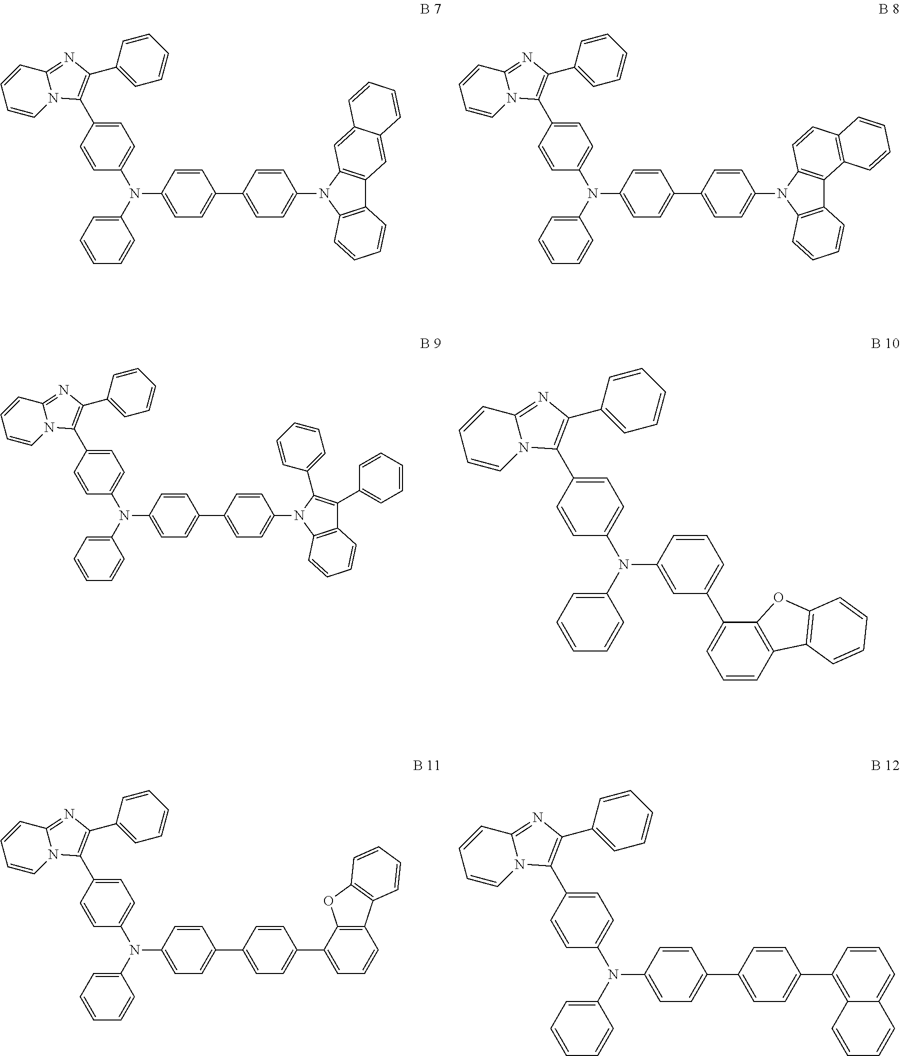

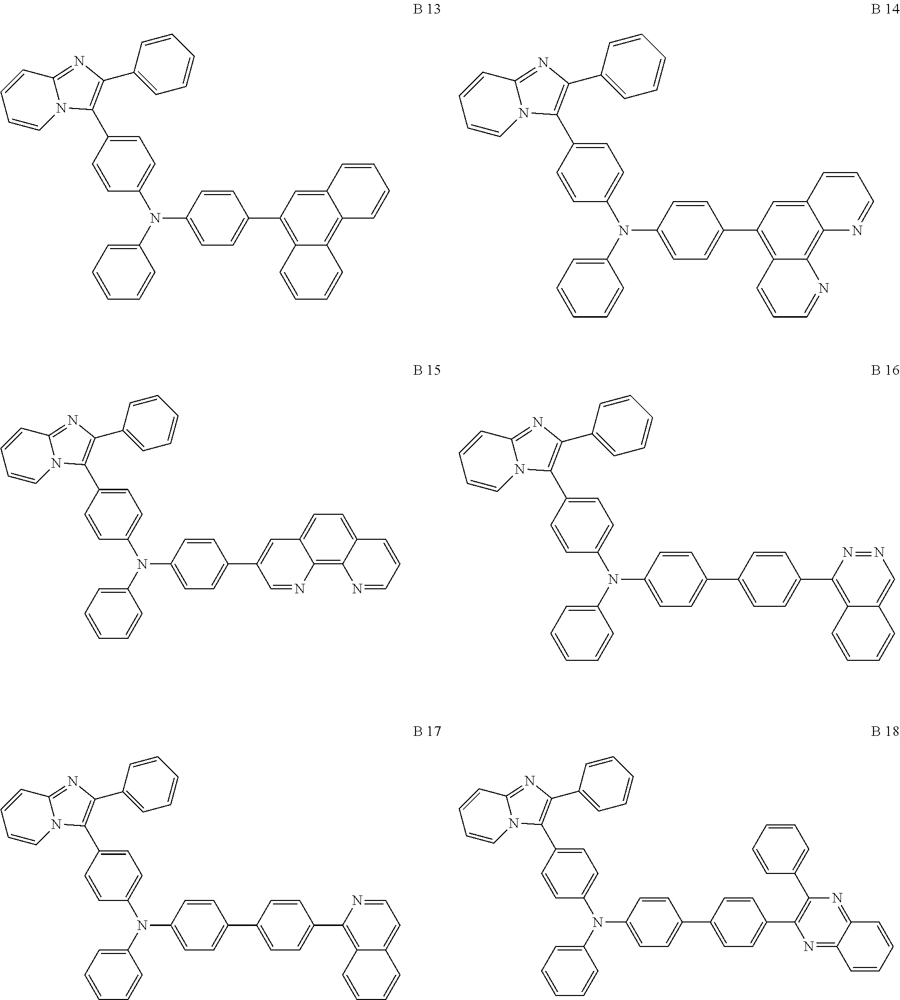

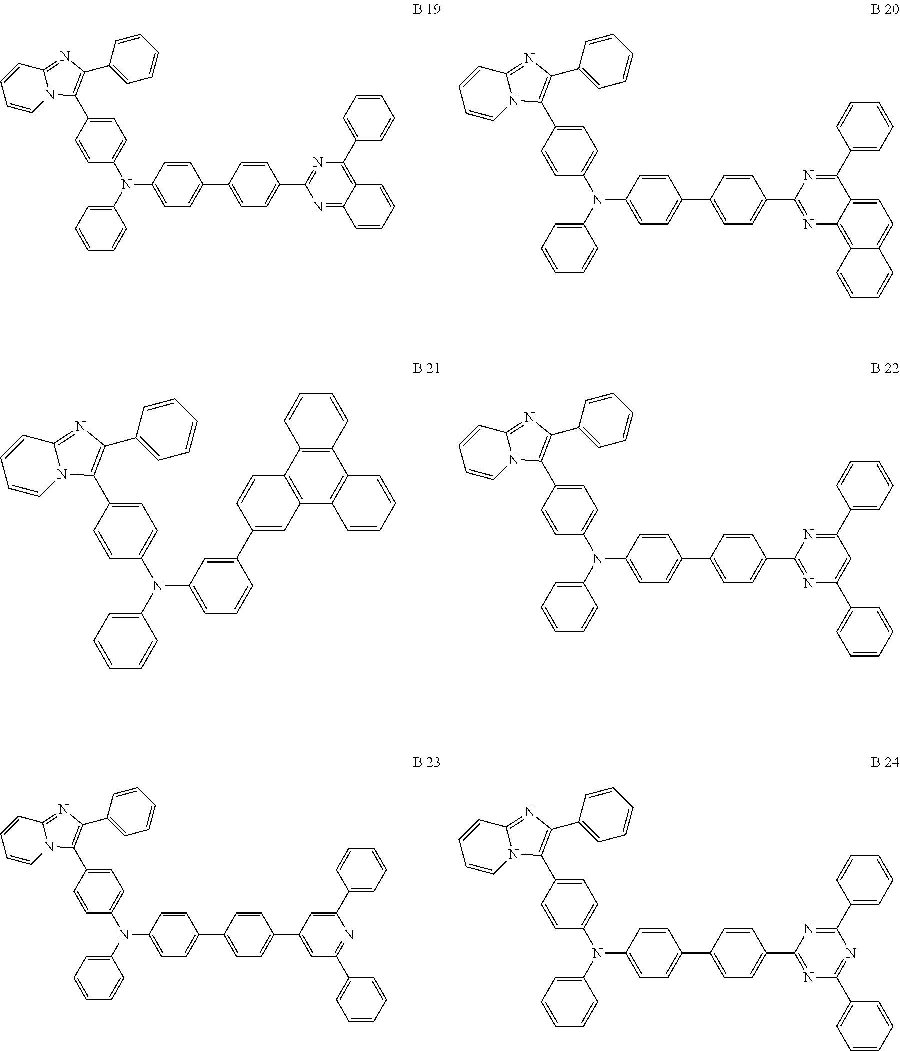

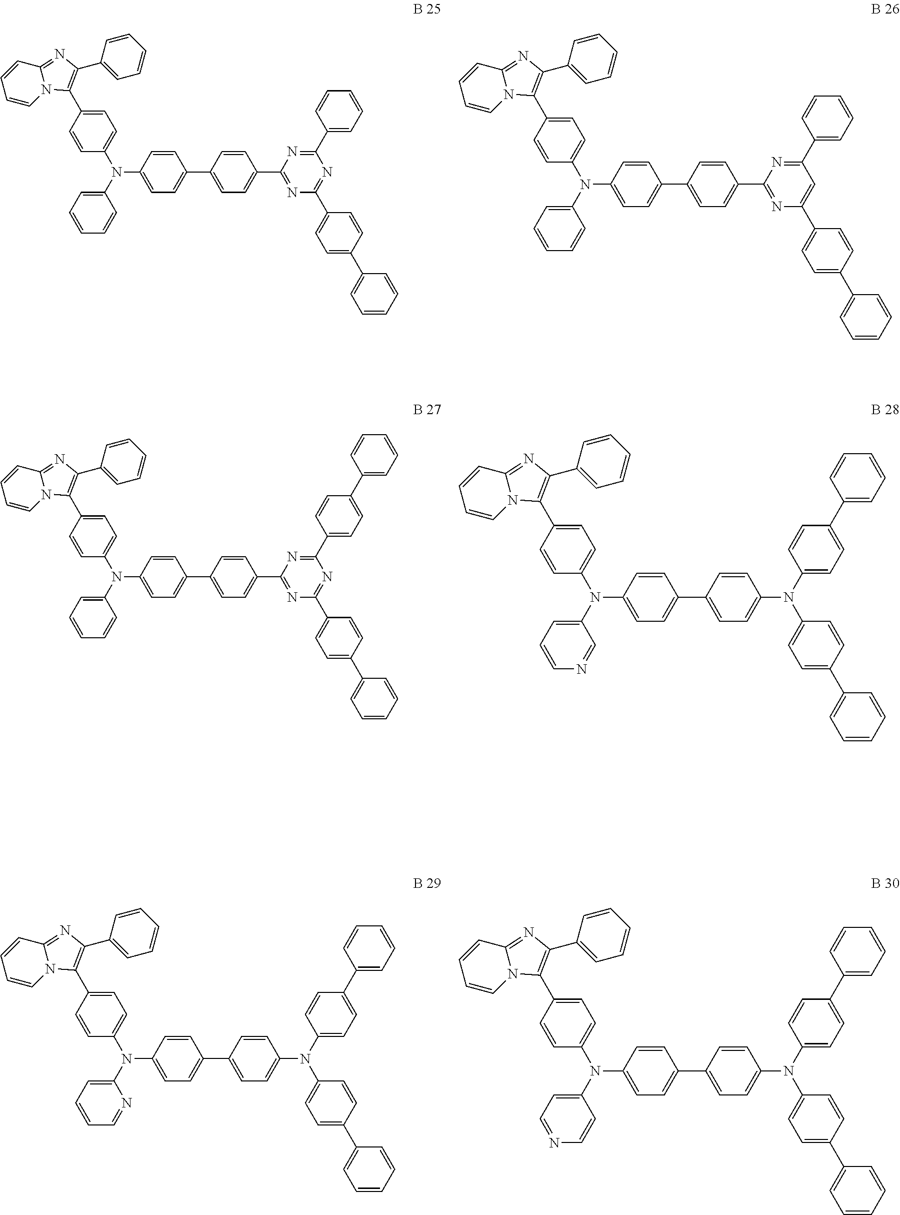

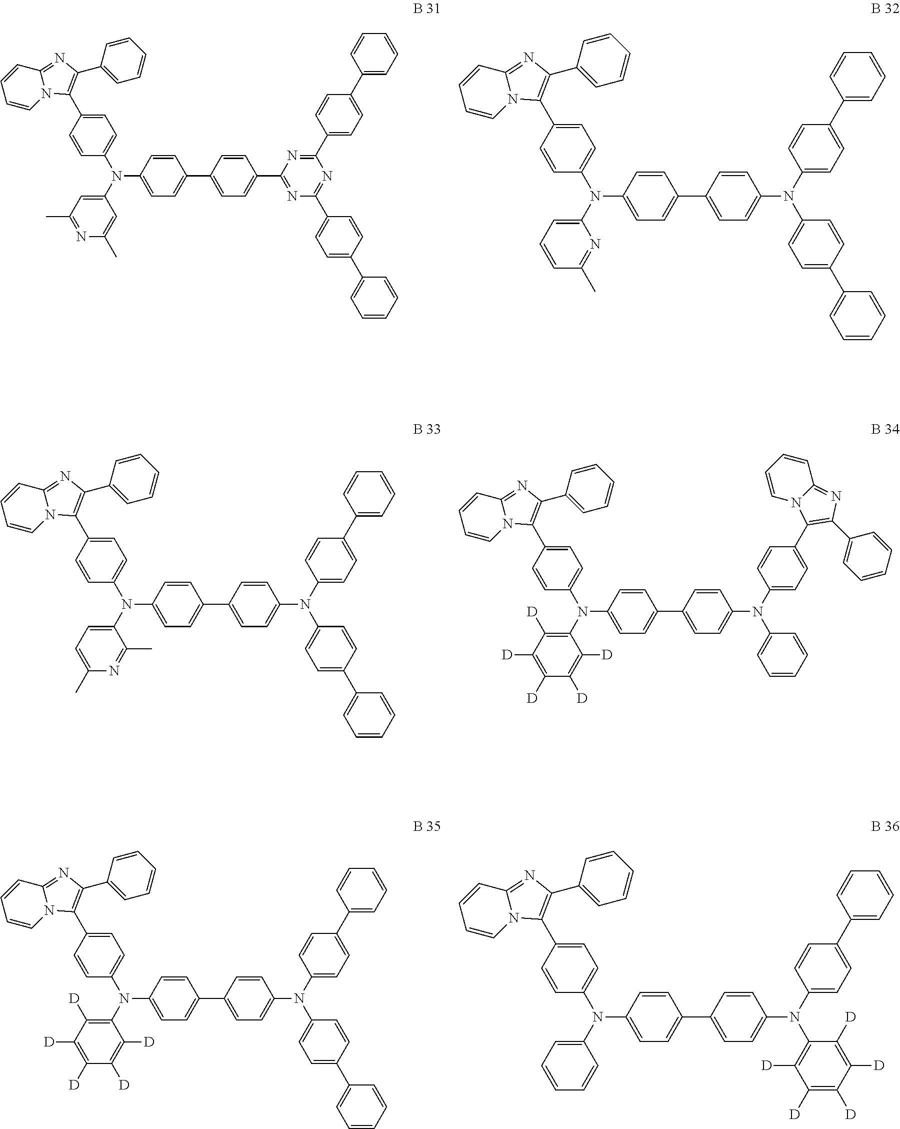

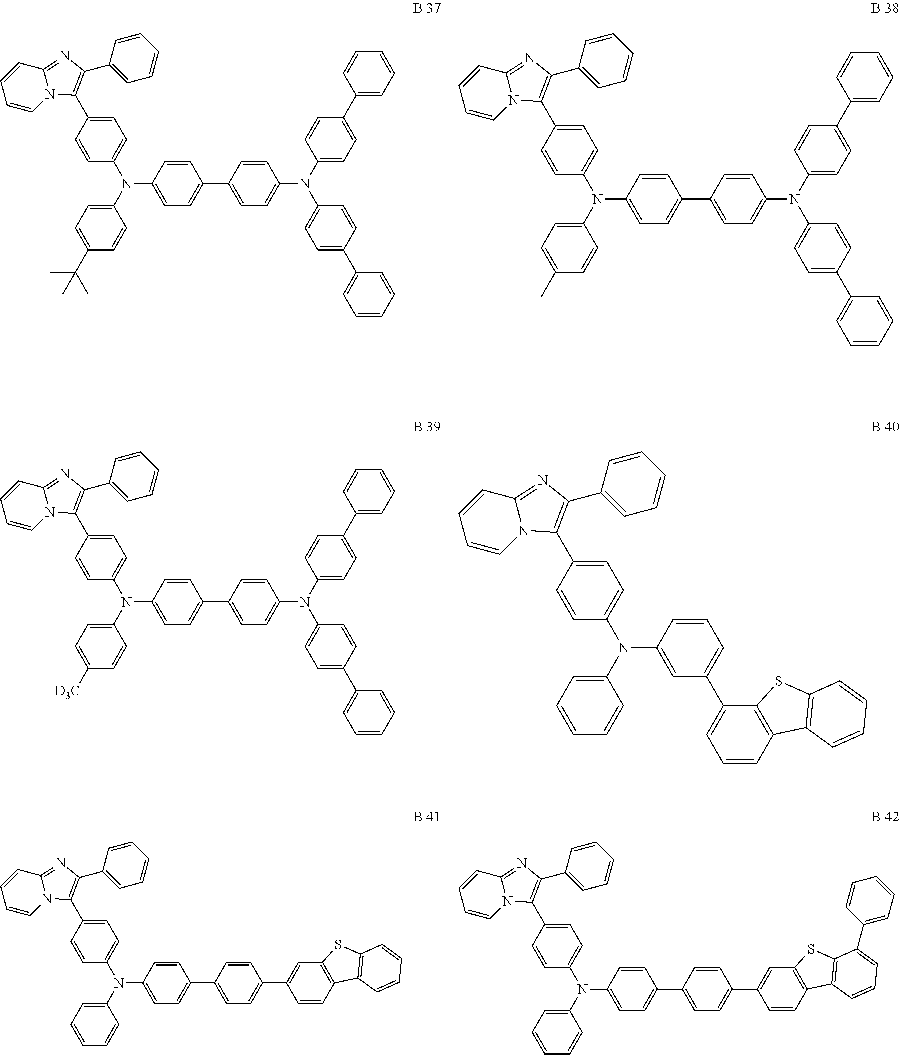

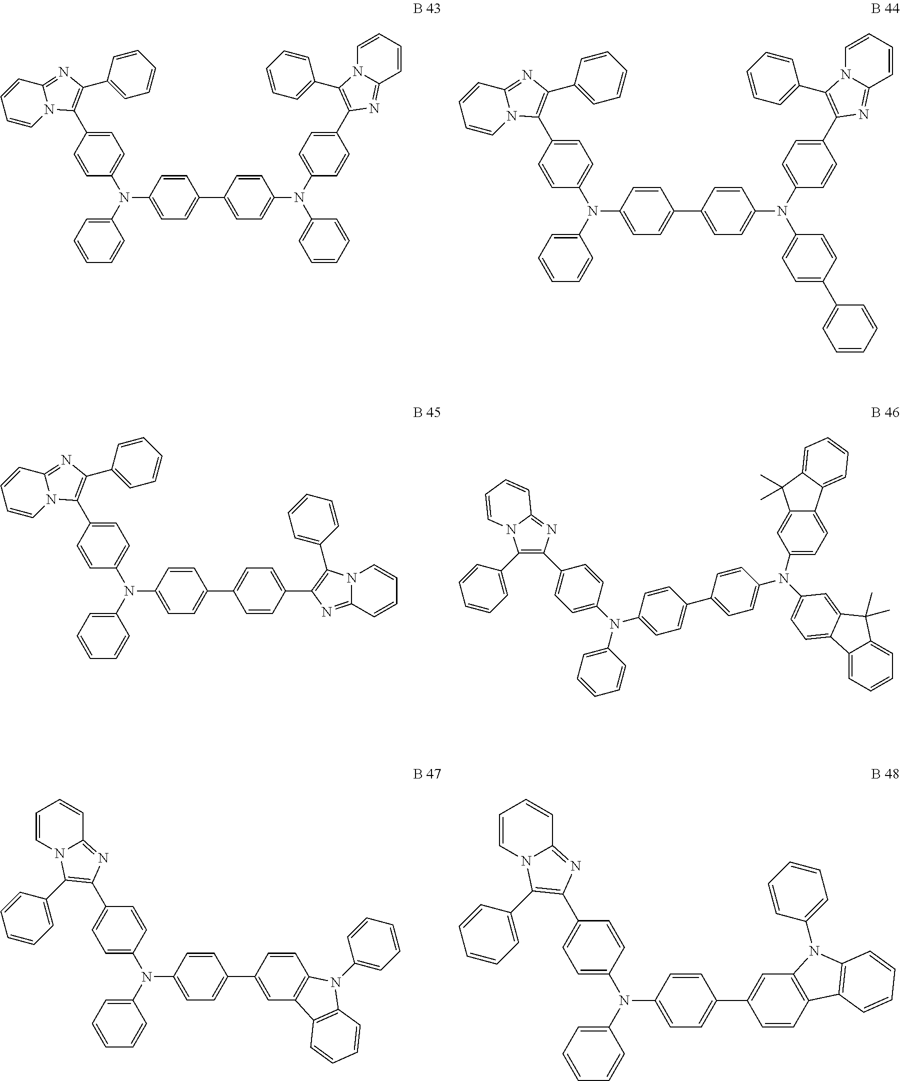

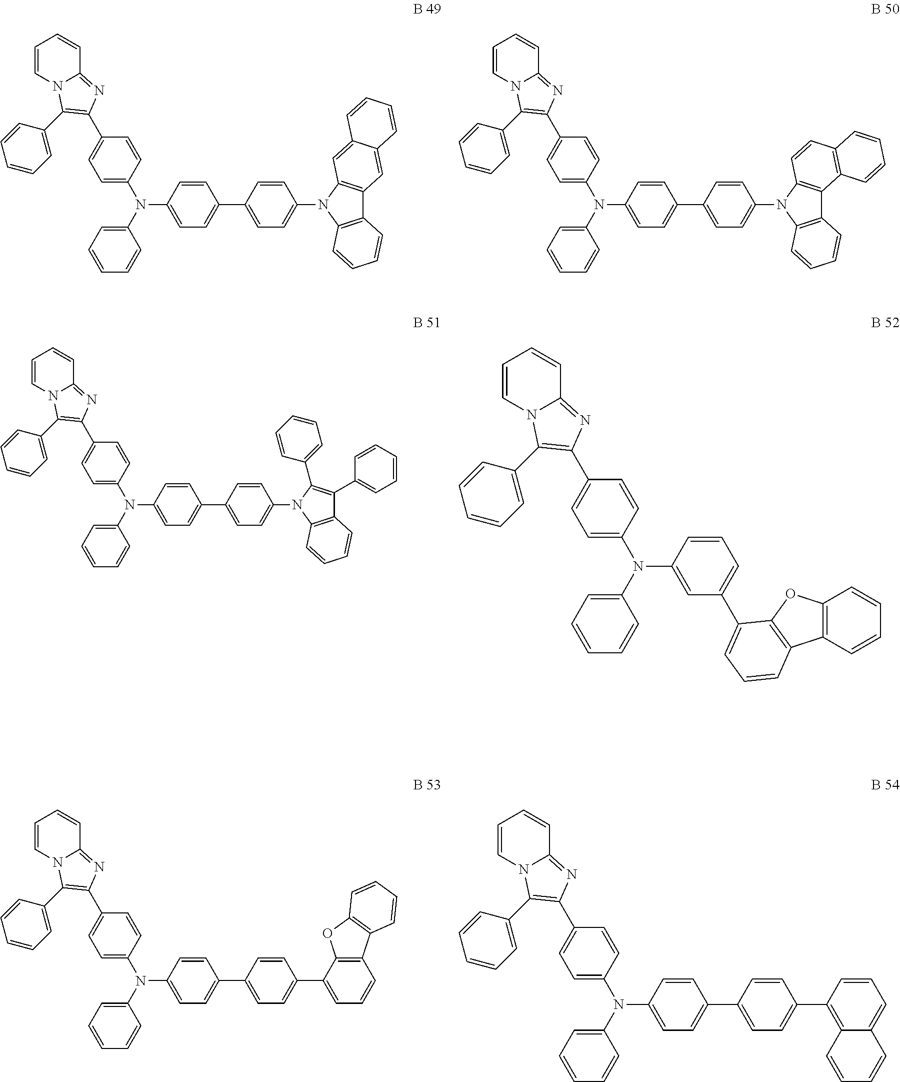

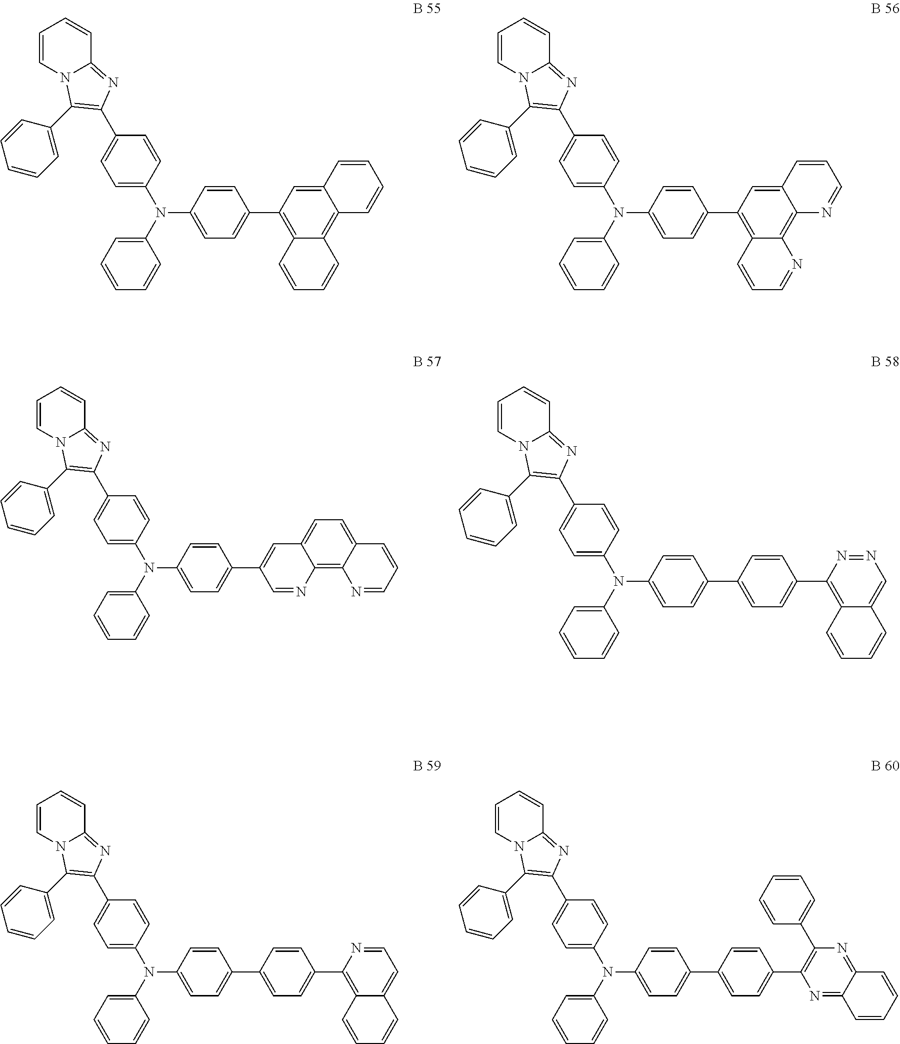

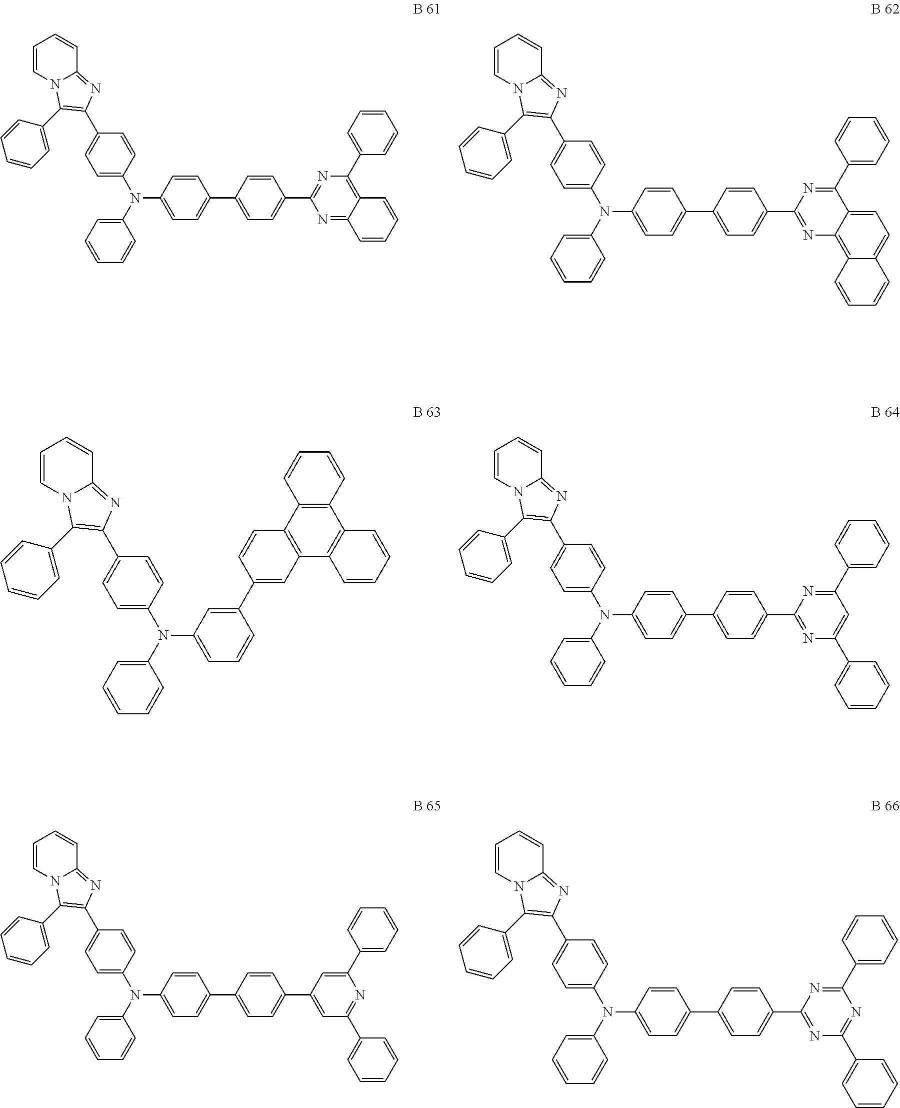

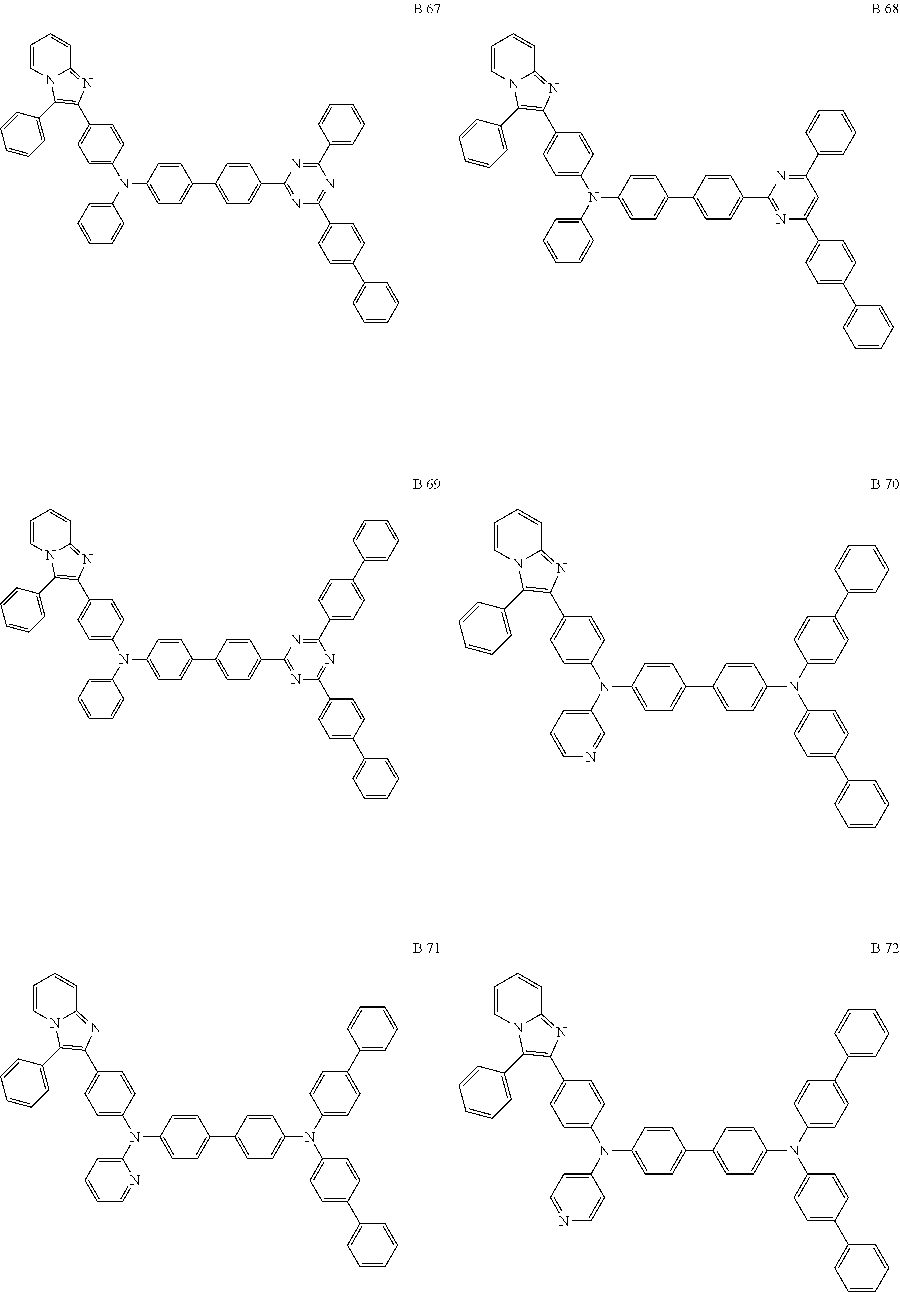

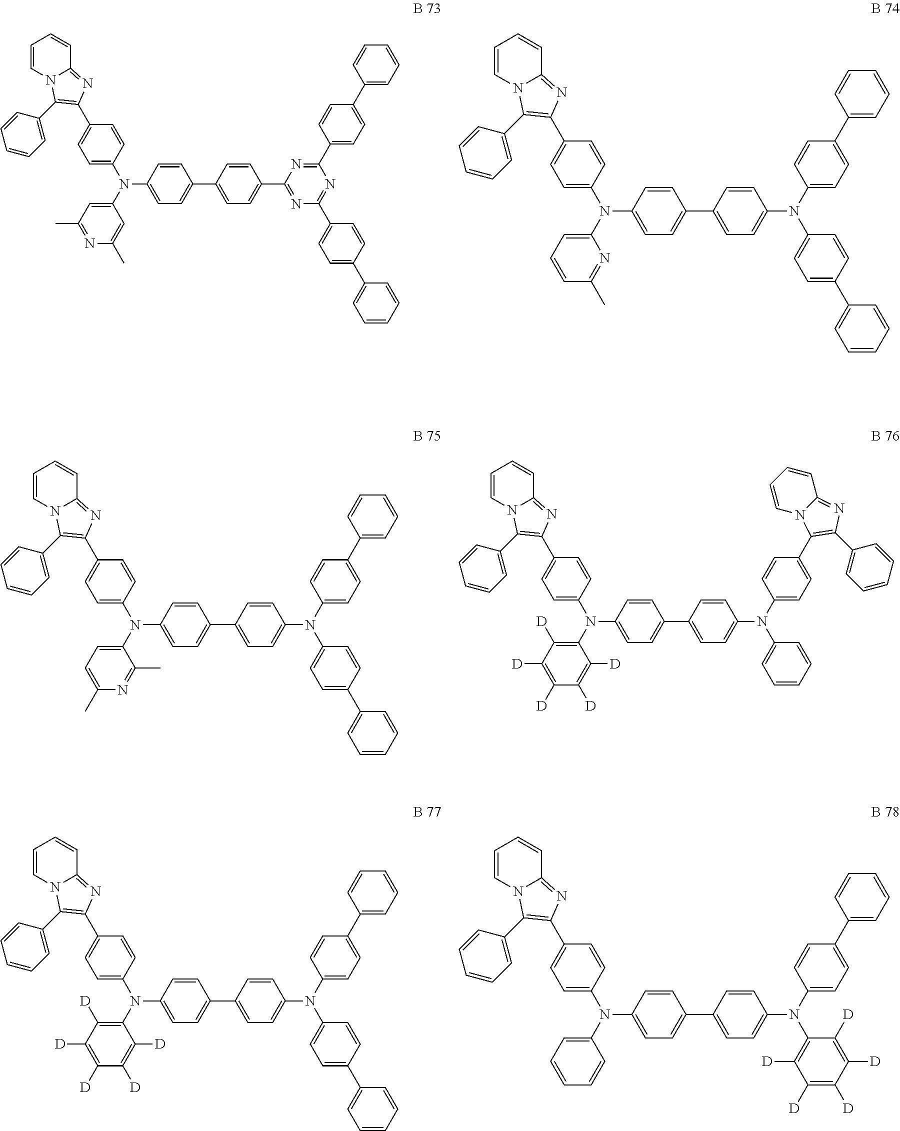

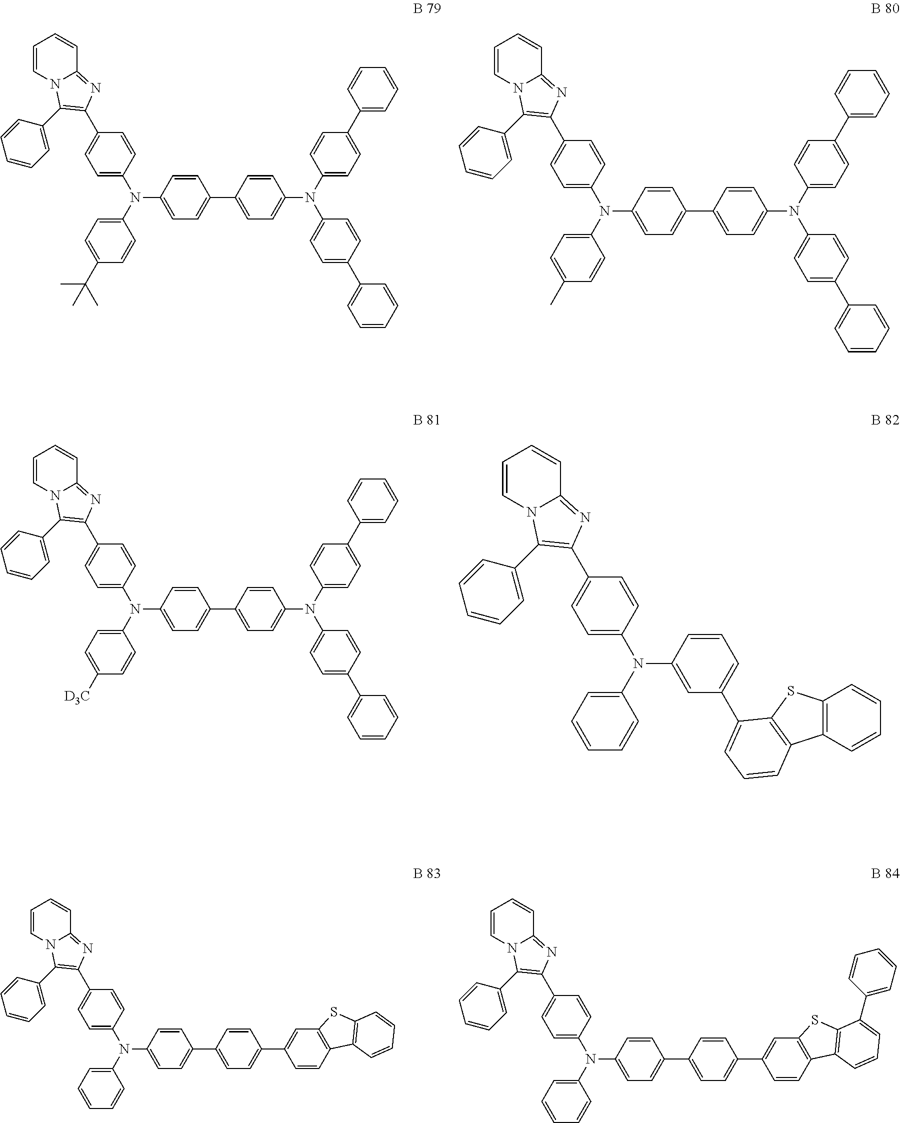

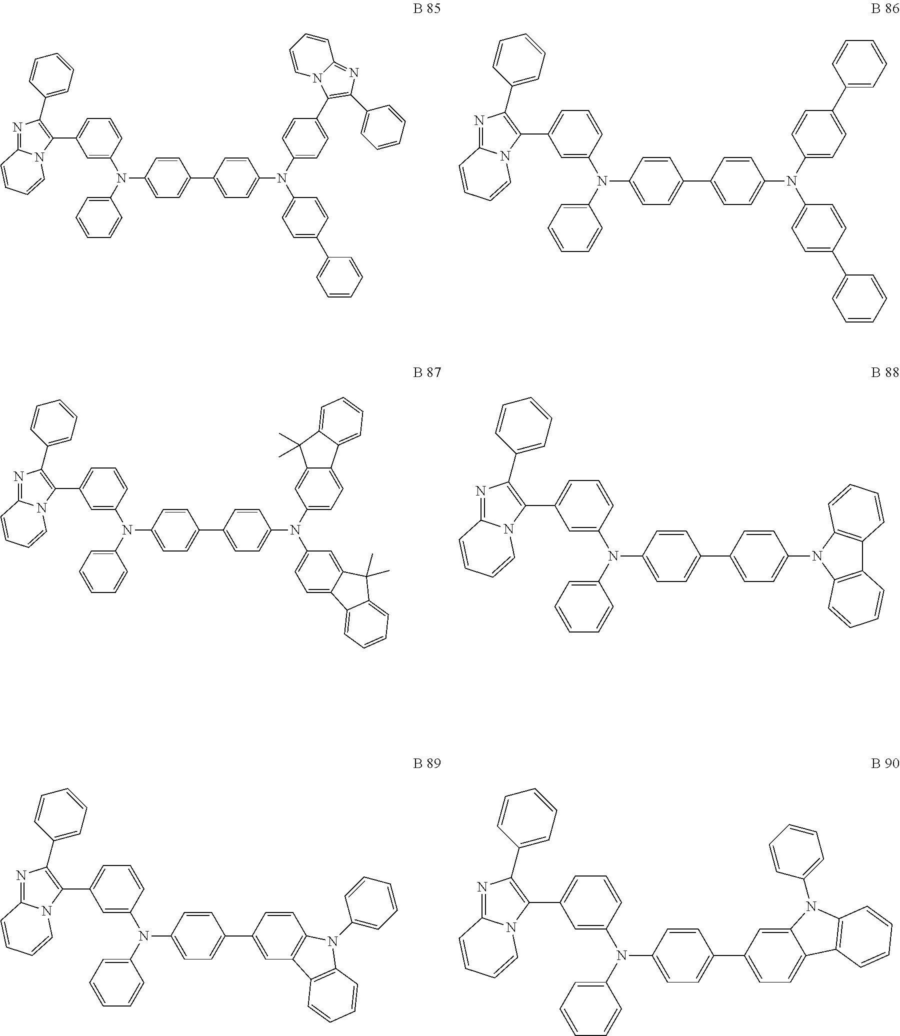

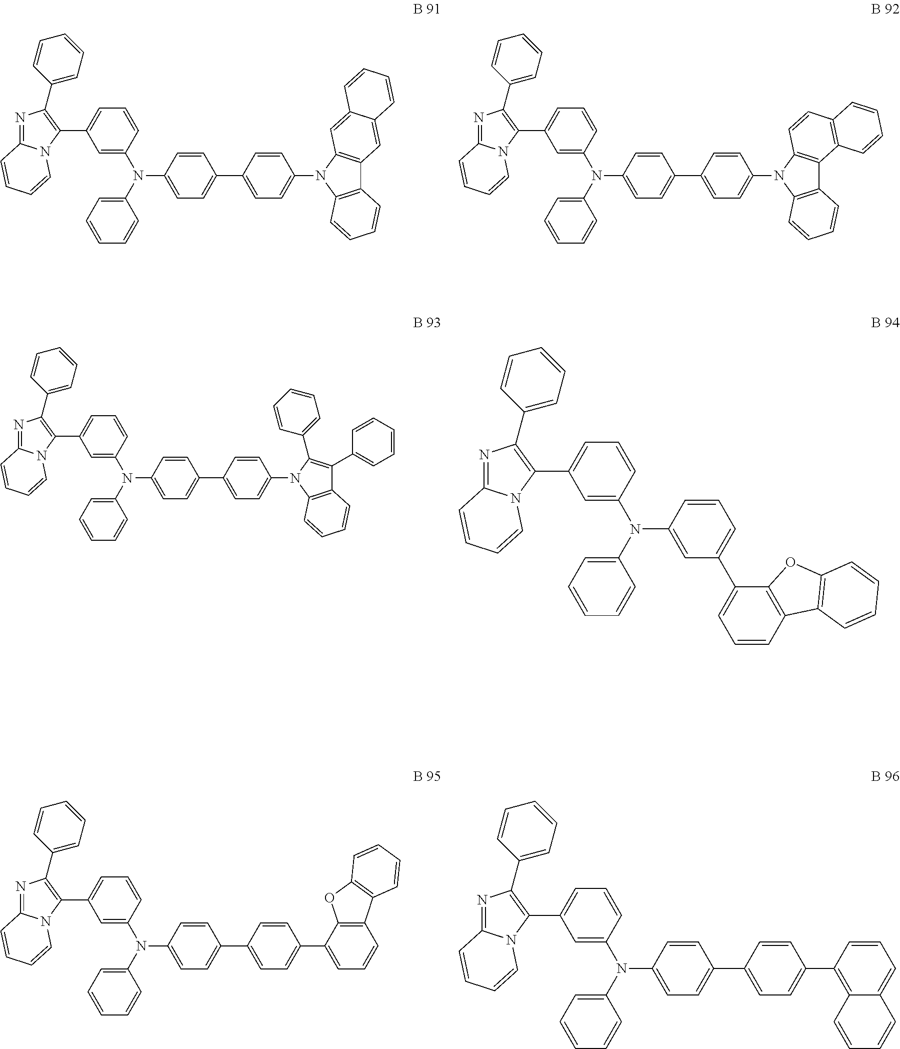

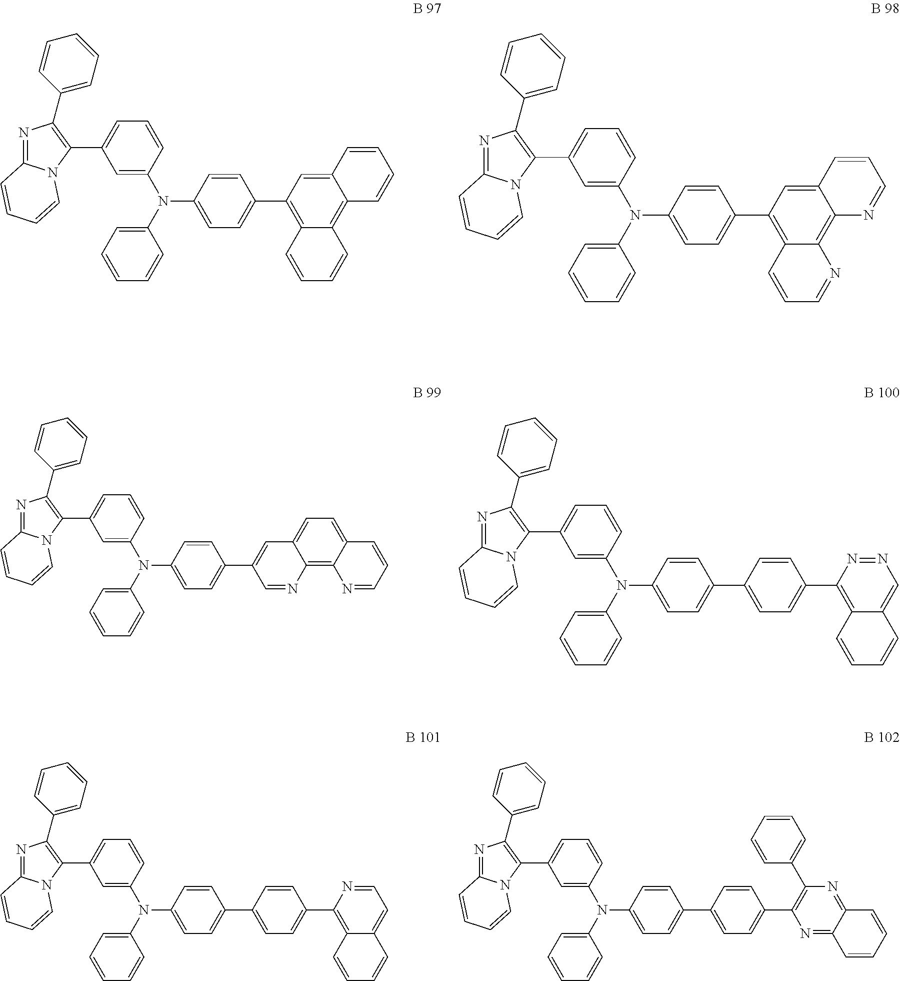

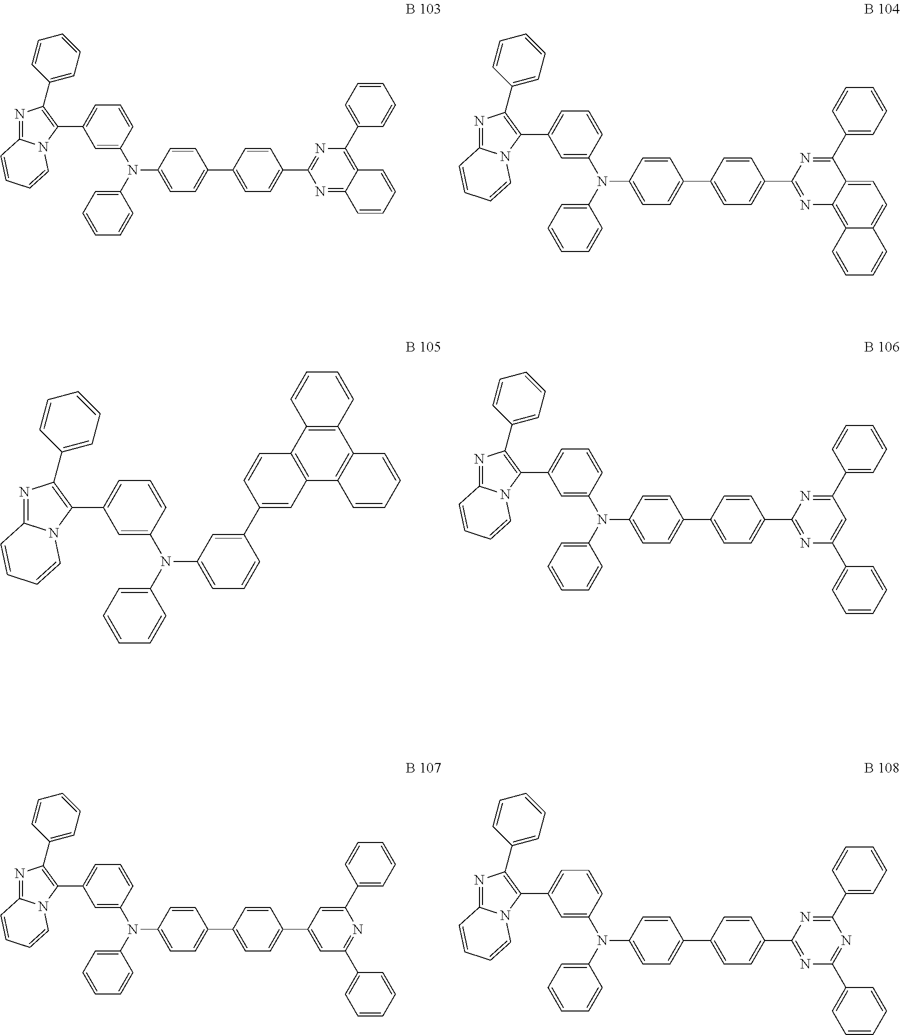

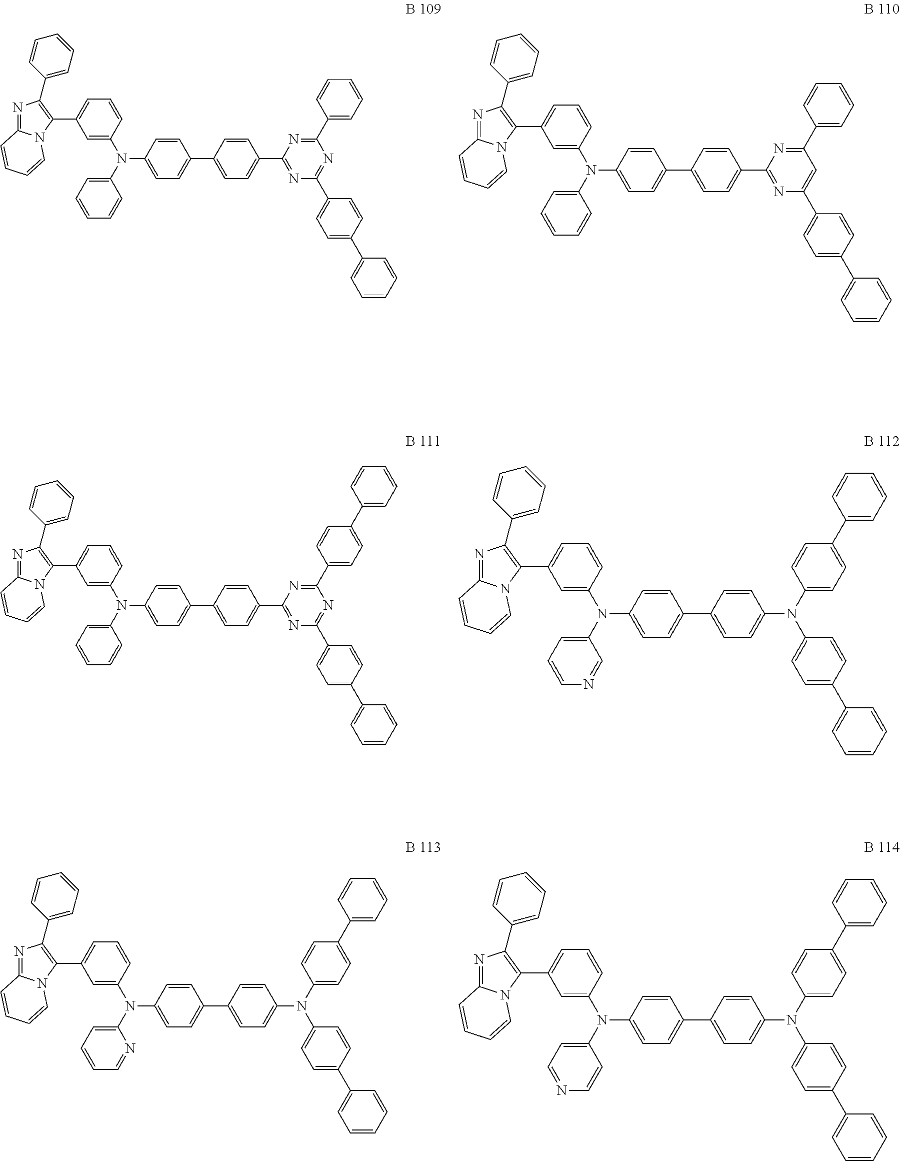

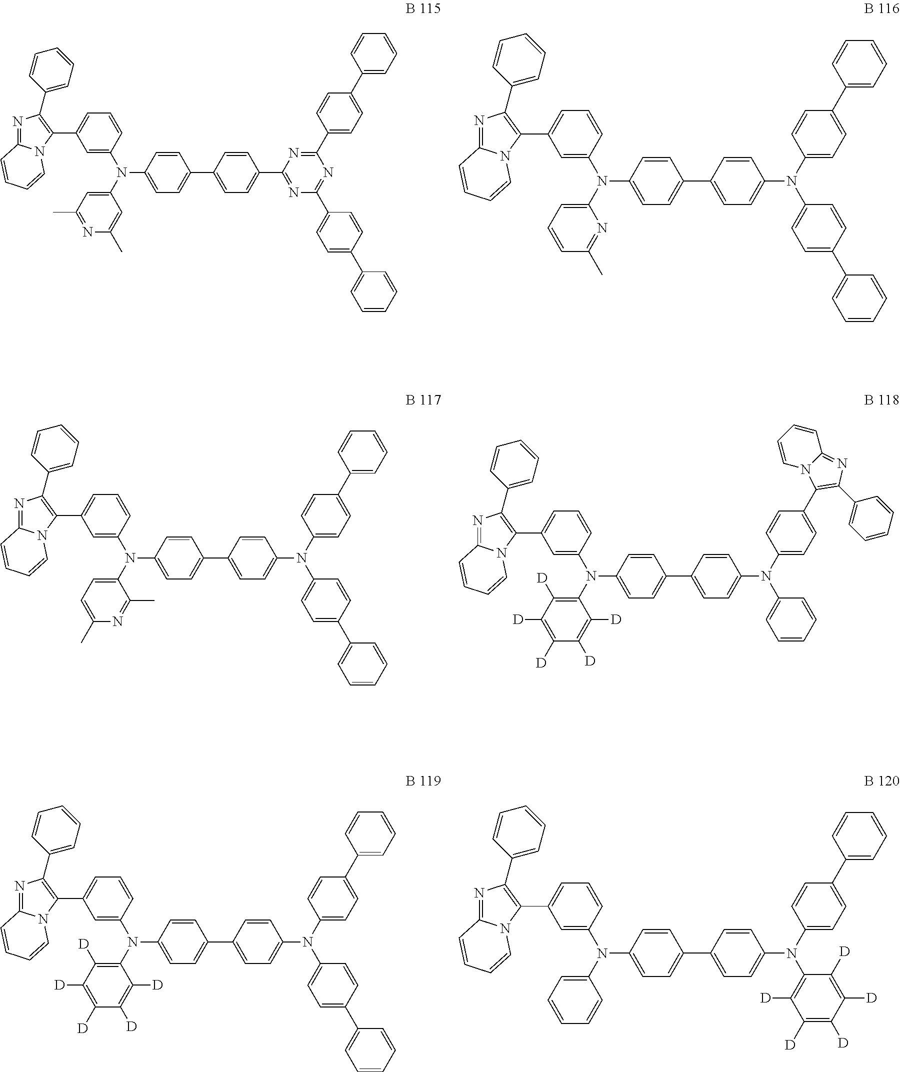

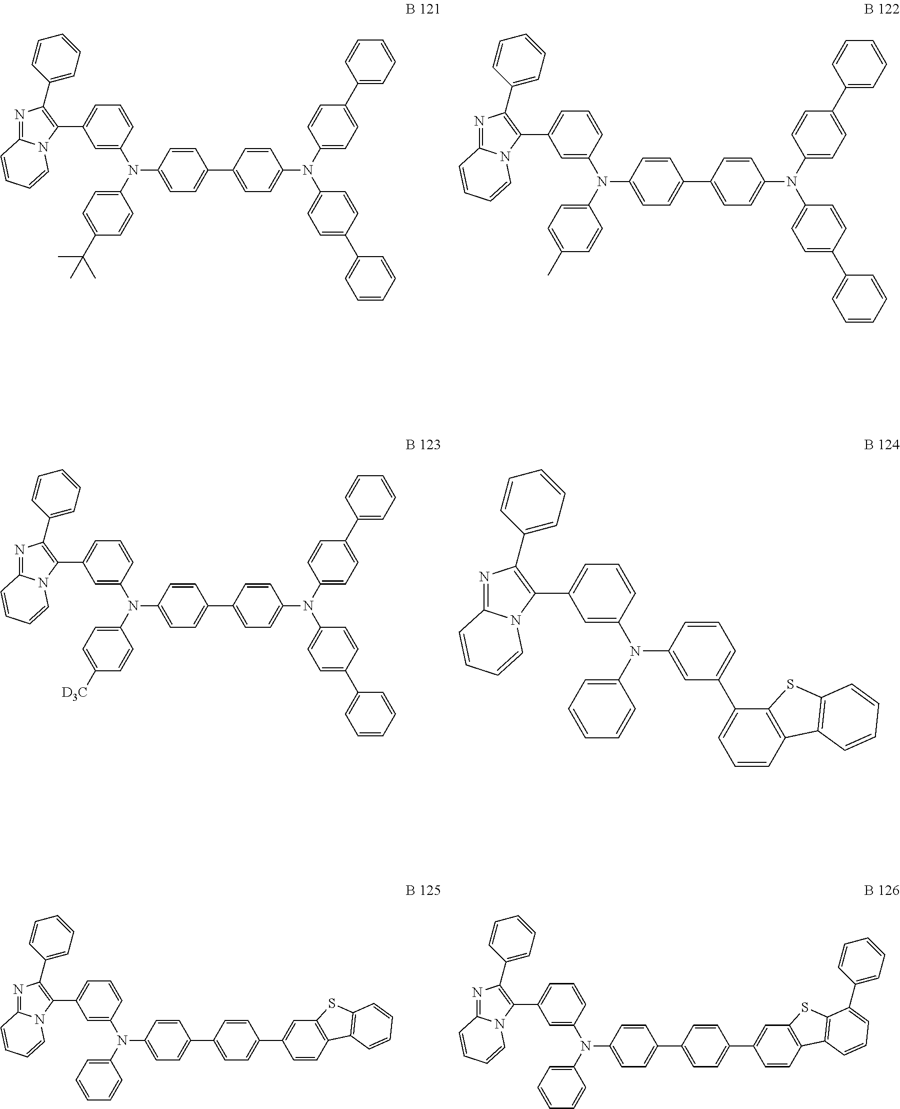

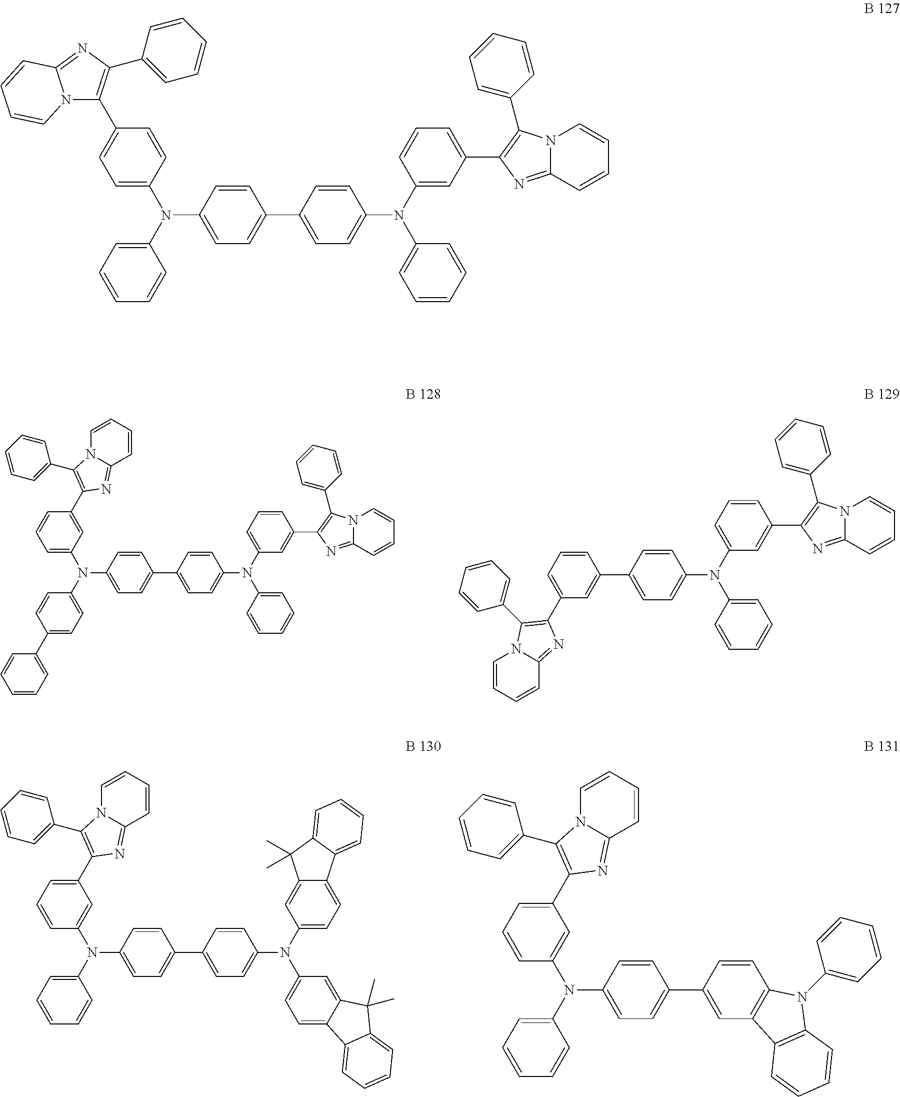

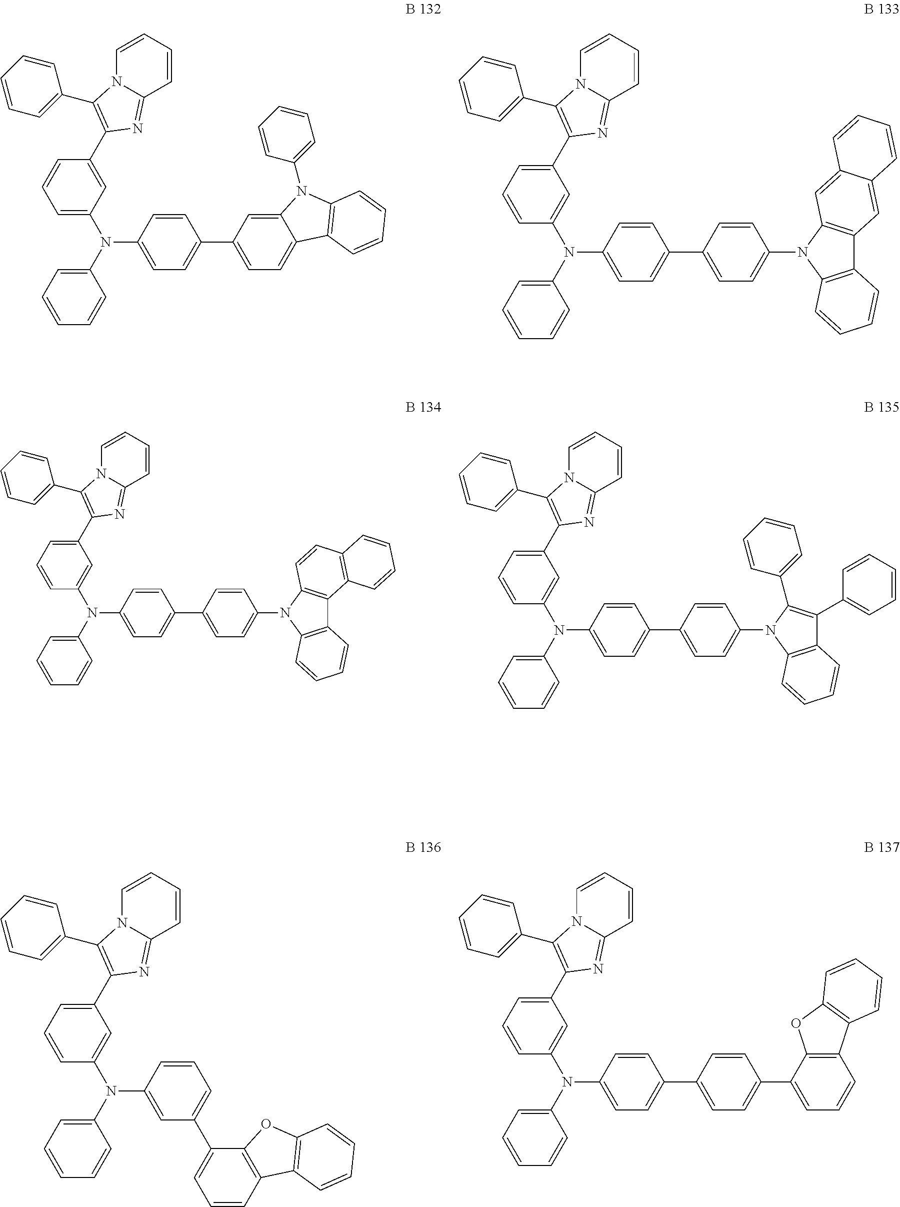

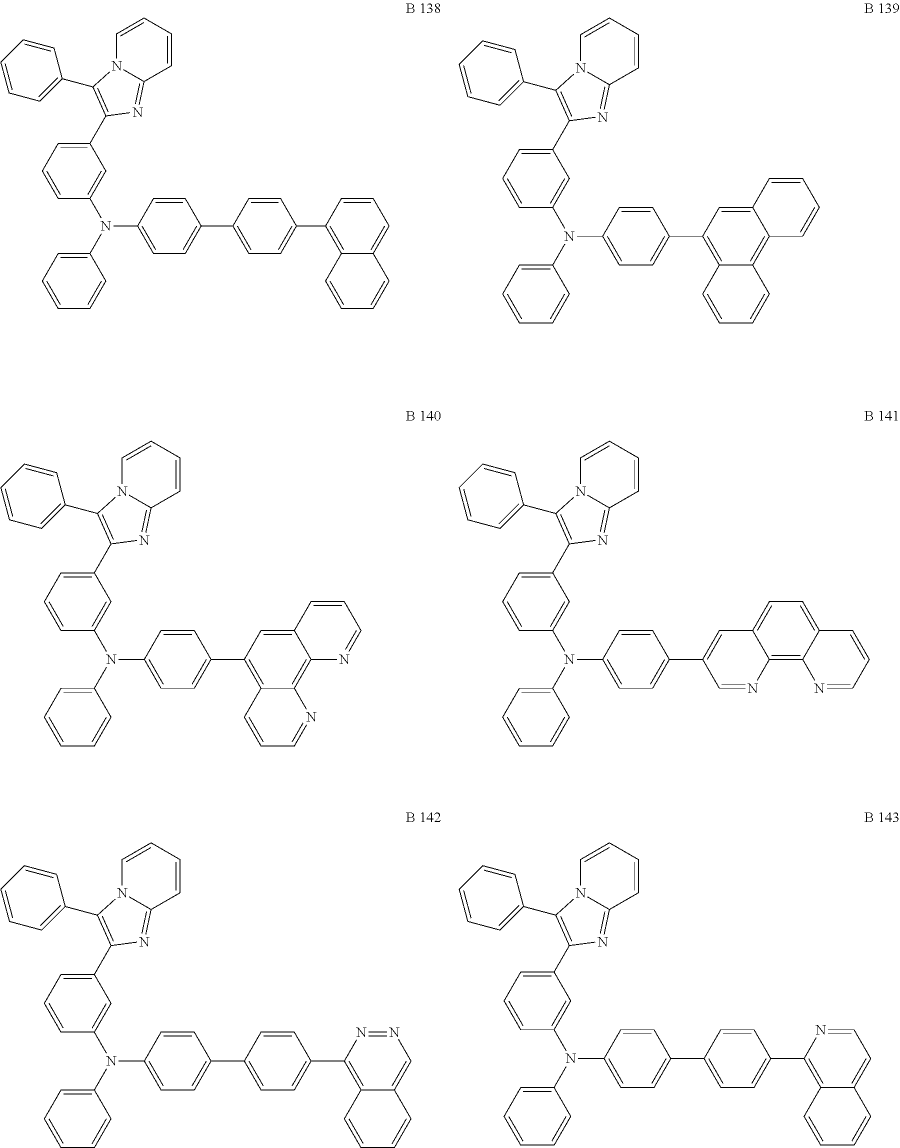

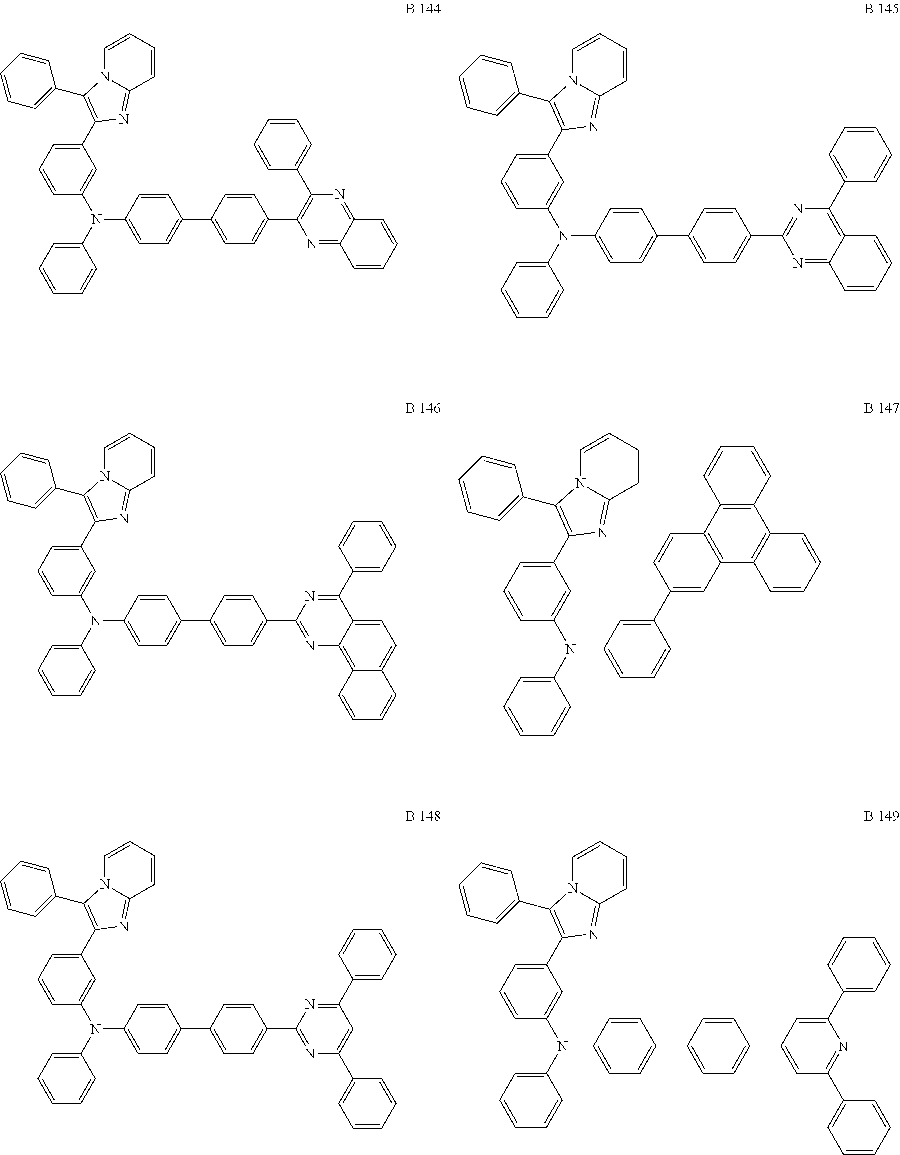

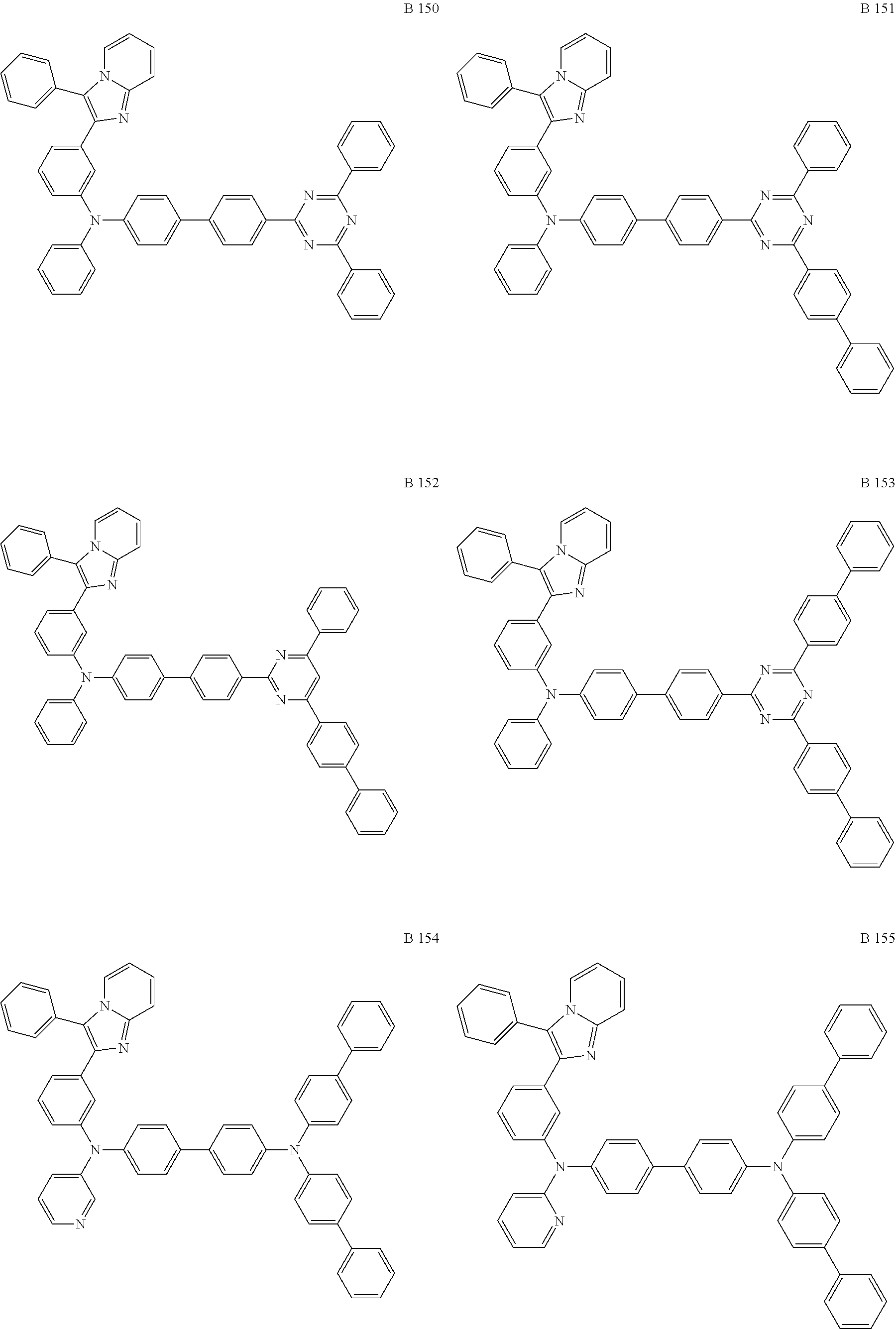

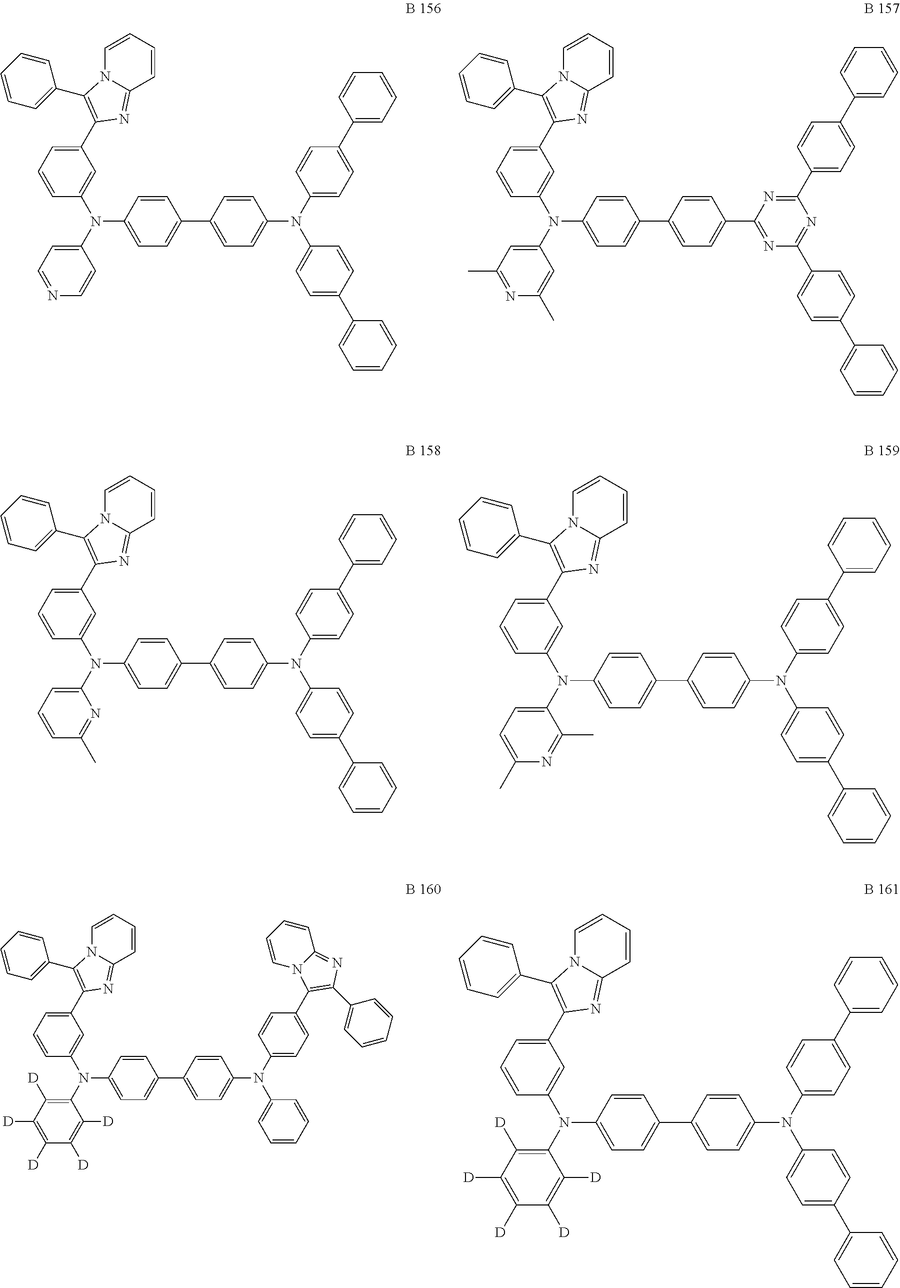

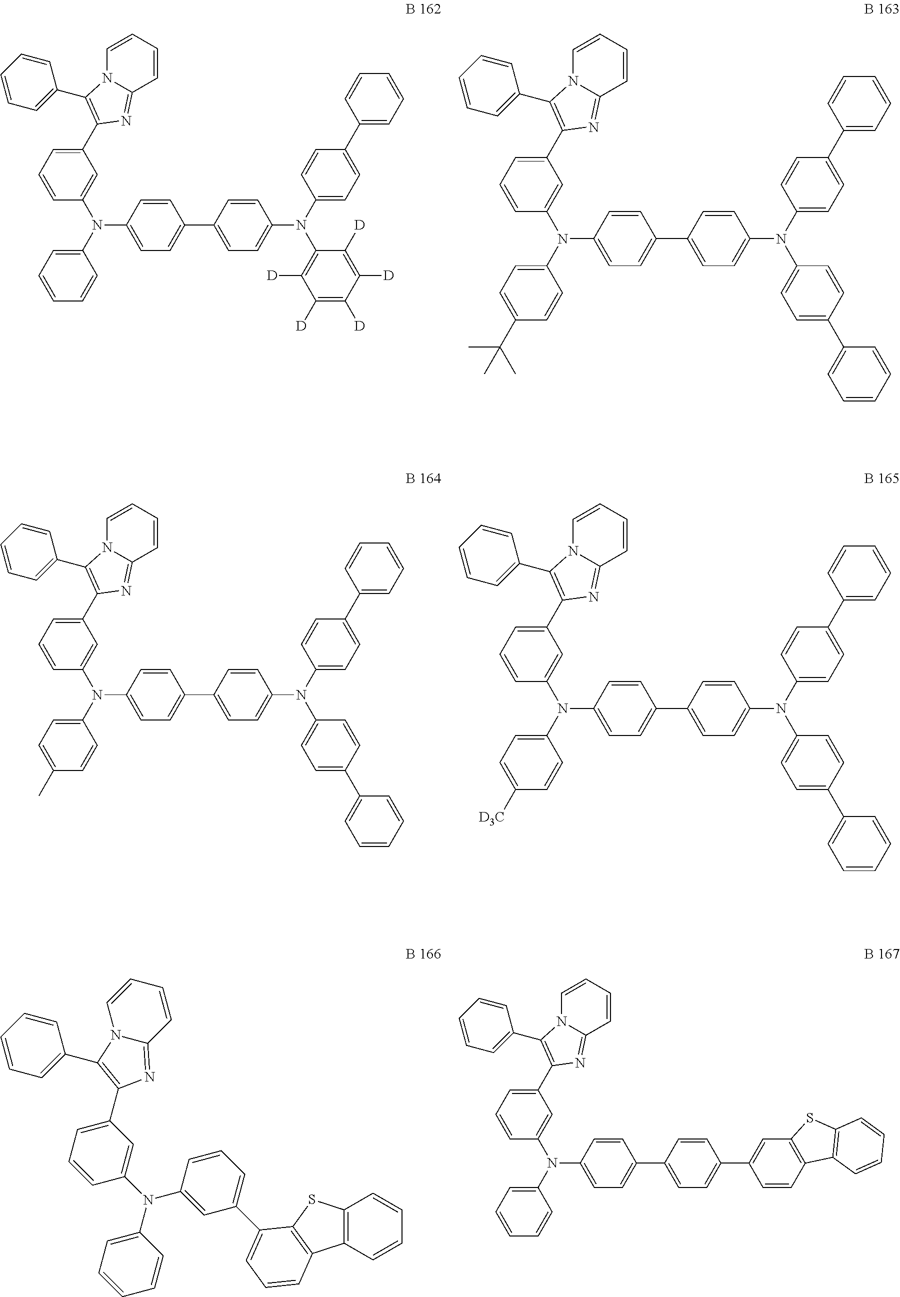

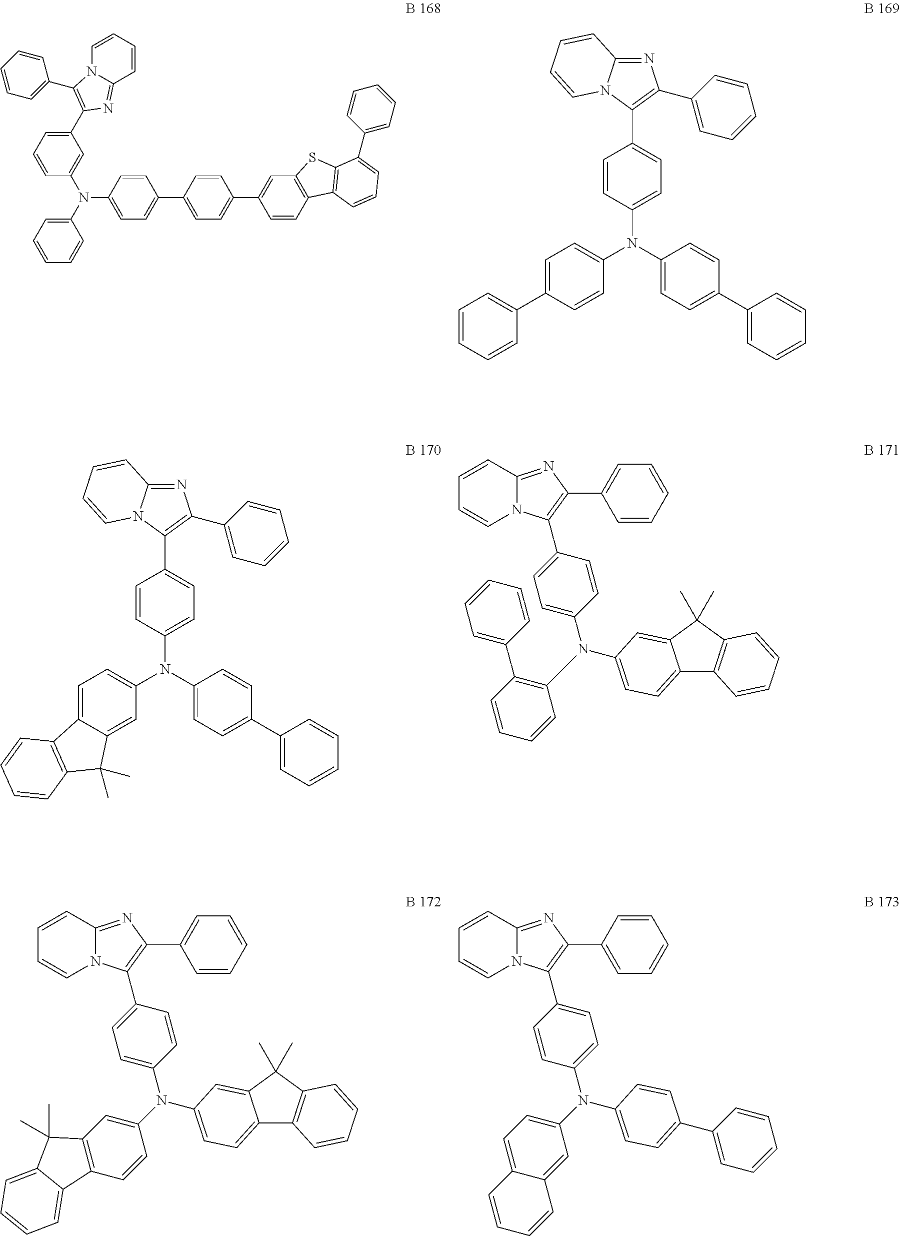

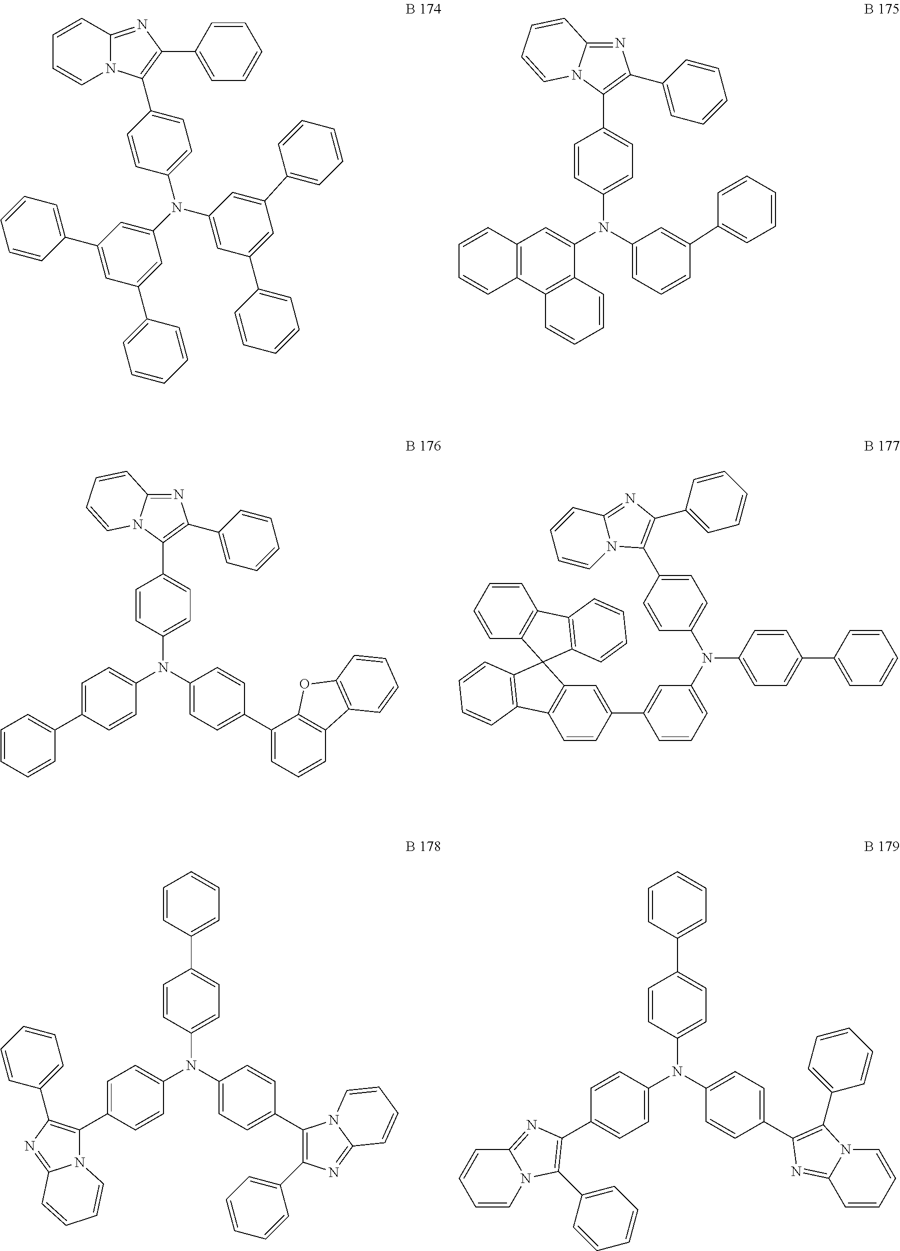

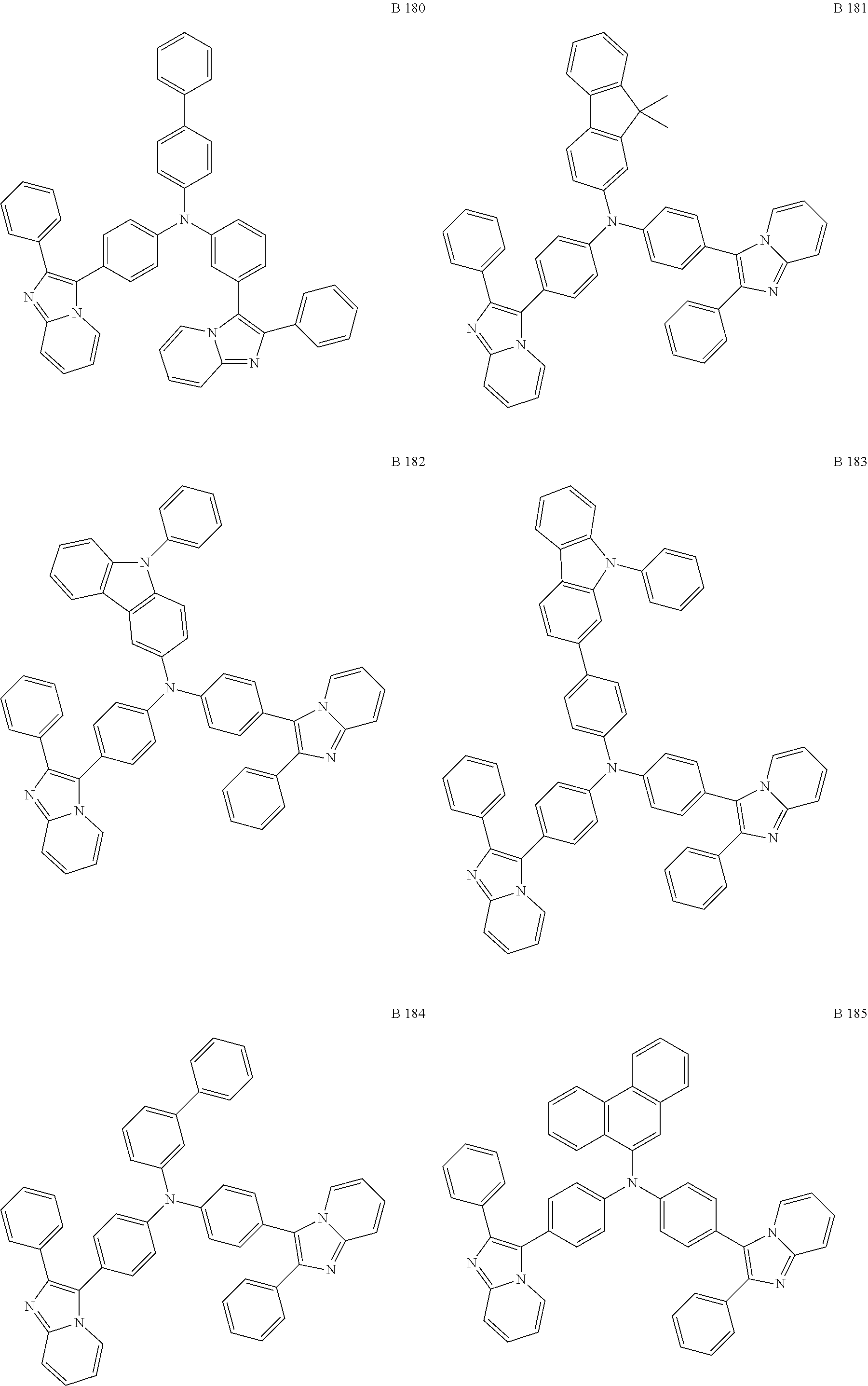

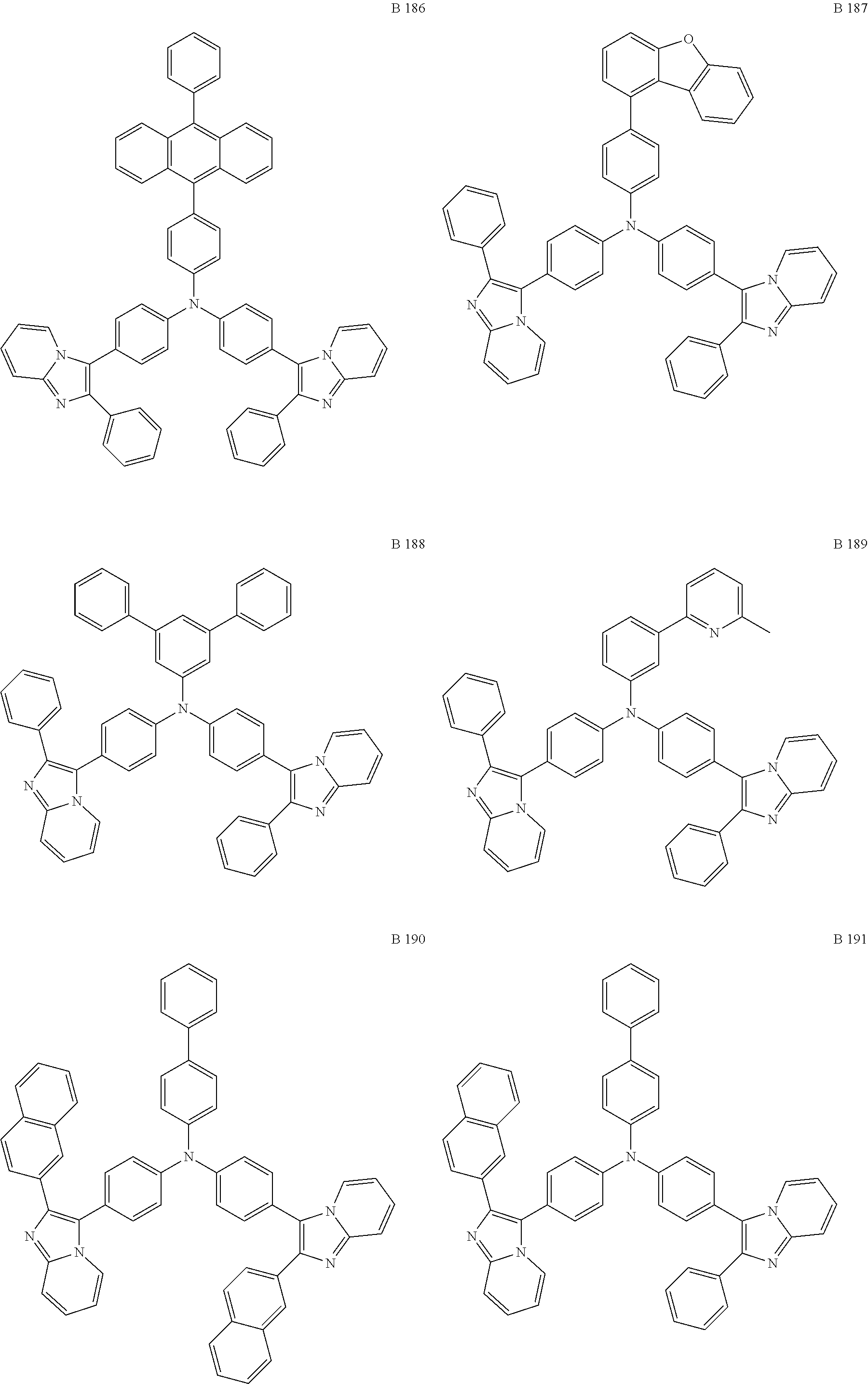

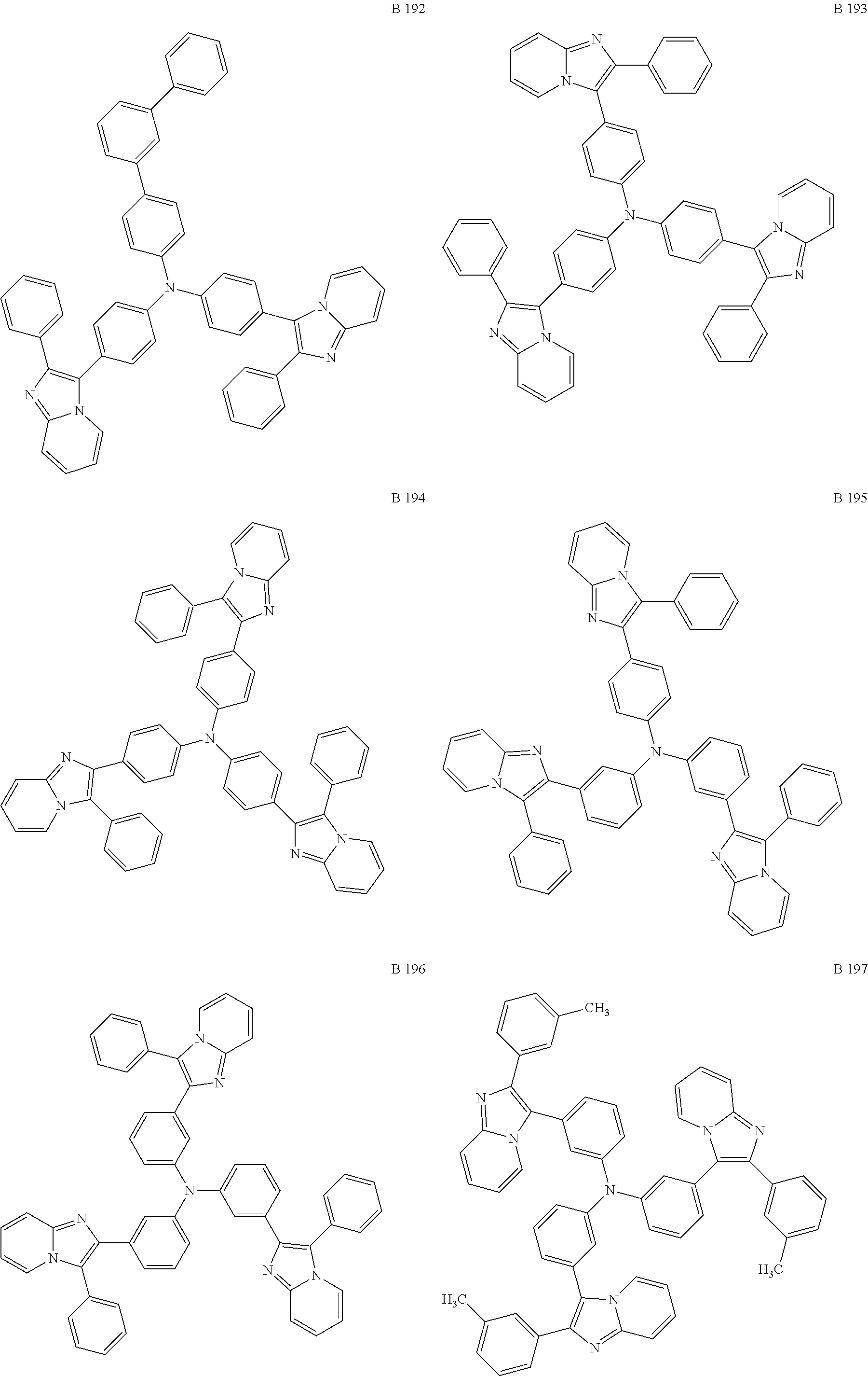

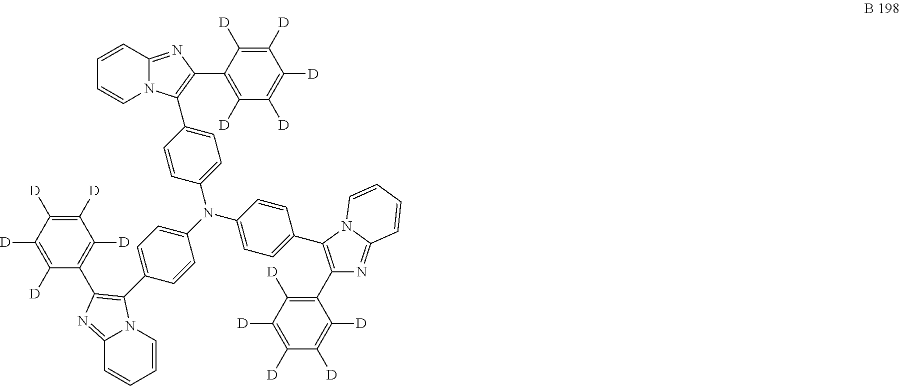 [SMILES: [2H]c1c([2H])c([2H])c(-c2nc3ccccn3c2-c2ccc(N(c3ccc(-c4c(-c5c([2H])c([2H])c([2H])c([2H])c5[2H])nc5ccccn45)cc3)c3ccc(-c4c(-c5c([2H])c([2H])c([2H])c([2H])c5[2H])nc5ccccn45)cc3)cc2)c([2H])c1[2H]]

The application is use of the compound as a light extraction layer material of an OLED device.

The imidazopyridine-based arylamine compound material of the present invention has the advantages such as, a low sublimation temperature, a good thermal stability, a high luminous efficiency, a high refractive index and a small refractive index difference in the visible light region, and can be used as to an organic light-emitting device. As a light extraction layer material, the device has the advantages of high luminous efficiency and good long-time heat stability and thus, has the possibility of being applied to AMOLED industry.

### EMBODIMENTS (IMPLEMENTATION FOR SYNTHESIS AND DEVICE)

The following examples are merely to facilitate the understanding of the present invention, but are not construed as specifically limiting the present invention.

Raw materials, solvents and the like related in the compound synthesis in the present invention are purchased from Alfa, Acros and other suppliers known well by a person skilled in the art.

### Example 1

### (1) Synthesis of the Compound A1

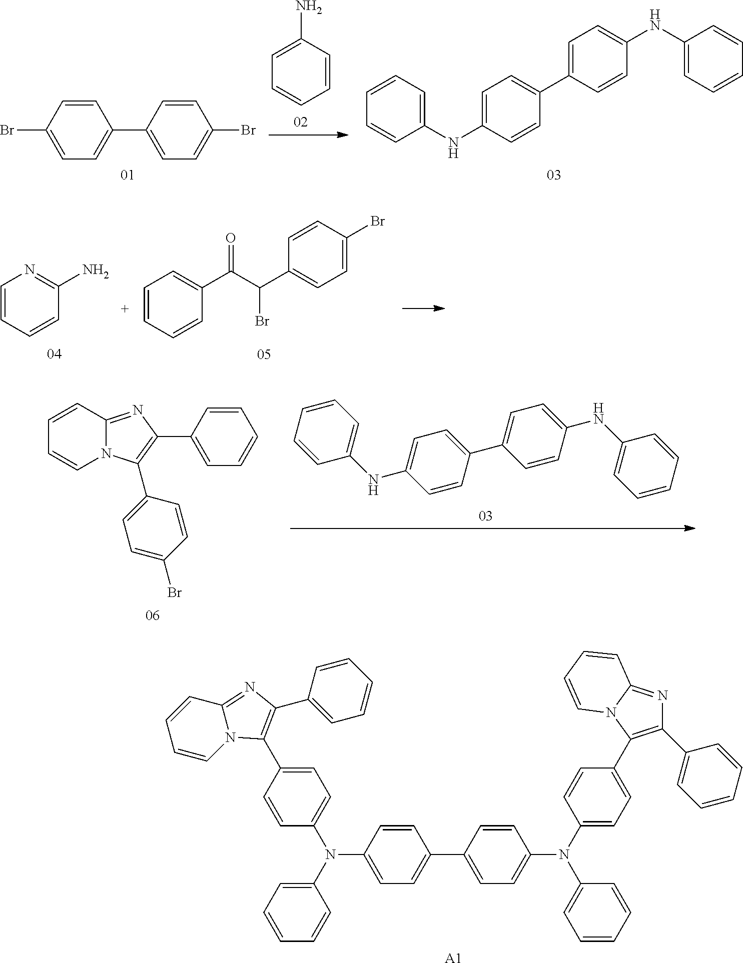

Synthesis of the compound 03: the compound 01 (80 g, 256.4 mmol, 1.0 eq), compound 02 (71.64 g, 769.2 mmol, 3.0 eq), t-BuONa (49.2 g, 512.8 mmol, 2.0 eq), Pd₂(dba)₃ (2.35 g, 2.56 mmol, 0.01 eq), X-phos (2.44 g, 5.13 mmol, 0.02 eq) and molecular sieve-dried toluene (800 ml) were successively fed into a 2 L three-necked flask, vacuumized and replaced with nitrogen for 3 times, then heated up to 108° C. around in an oil bath and stirred for 2 h at reflux under heat preservation; sampling was performed, and completion of the reaction of the raw material 01 was monitored by TLC. The reaction liquid was cooled to 80° C., and dropwisely added methanol (800 ml) and stirred for 1 h, then cooled to room temperature and subjected to suction filtration; the obtained solid was added THF (900 ml) and n-hexane (600 ml) for hot beating for 2 h, and subjected to suction filtration and dried to obtain 71.5 g of an off-white solid with a yield of 82.8%. Mass spectrometry: 337.4 (M+H)

Synthesis of the compound 06: the compound 04 (76 g, 214.6 mmol, 1.0 eq), compound 05 (50.5 g, 536.6 mmol, 2.5 eq), NaHCO₃ (27.05 g, 322 mmol, 1.5 eq) and isopropanol (700 ml) were successively fed into a 2 L single-necked flask, then heated up to 80° C. around in an oil bath and stirred for 7 h at reflux under heat preservation; sampling was performed, and completion of the reaction of the raw material was monitored by TLC. The system was cooled and dropwisely added deionized water, stirred for 2 h around and then subjected to suction filtration. Solid was beaten by ethyl acetate and subjected to suction filtration and drying, thus obtaining 54.5 g of a white solid compound 06 with a yield of 72.7%. Mass spectrometry: 349.2 (M+H)

Synthesis of the compound A1: the compound 06 (45.7, 130.8 mmol, 2.2 eq), compound 03 (20 g, 59.4 mmol, 1 eq), t-BuONa (17.1 g, 178.3 mmol, 3.0 eq), Pd₂(dba)₃ (1.09 g, 1.19 mmol, 0.02 eq), X-phos (1.13 g, 2.38 mmol, 0.04 eq) and molecular sieve-dried toluene (600 ml) were successively fed into a 2 L three-necked flask, vacuumized and replaced with nitrogen for 3 times, then heated up to 108° C. around in an oil bath and stirred for 16 h at reflux under heat preservation; sampling was performed, and completion of the reaction of the raw material 06 was monitored by TLC. The reaction liquid was cooled to 80° C., and dropwisely added n-hexane (800 ml) and stirred for 1 h, then cooled to room temperature and subjected to suction filtration; the obtained solid was added dichloromethane (1.6 L) to be dissolved fully, then washed for four times (500 ml*4) with deionized water; after liquid separation, organic phases were filtered by silica gel; and the silica gel was washed with a small amount of dichloromethane, then the organic phases were concentrated to obtain a solid; and the solid was recrystallized with tetrahydrofuran/methanol (250 ml/300 ml) twice, and dried to obtain 32 g of a light yellow solid with a yield of 61.6%. The obtained composite was sublimated and purified to obtain 21.2 g of light yellow solid compound A1 with a yield of 66.2%. Mass spectrometry: 874.1 (M+H).

¹HNMR (400 MHz, CDCl₃) δ 8.01 (d, J=6.8 Hz, 2H), 7.72 (d, J=8.0 Hz, 3H), 7.64 (d, J=9.0 Hz, 2H), 7.51 (d, J=8.3 Hz, 4H), 7.40-7.13 (m, 23H), 7.10 (d, J=7.2 Hz, 2H), 6.74 (d, J=6.8 Hz, 2H).

### (2) Synthesis of the Compound A16

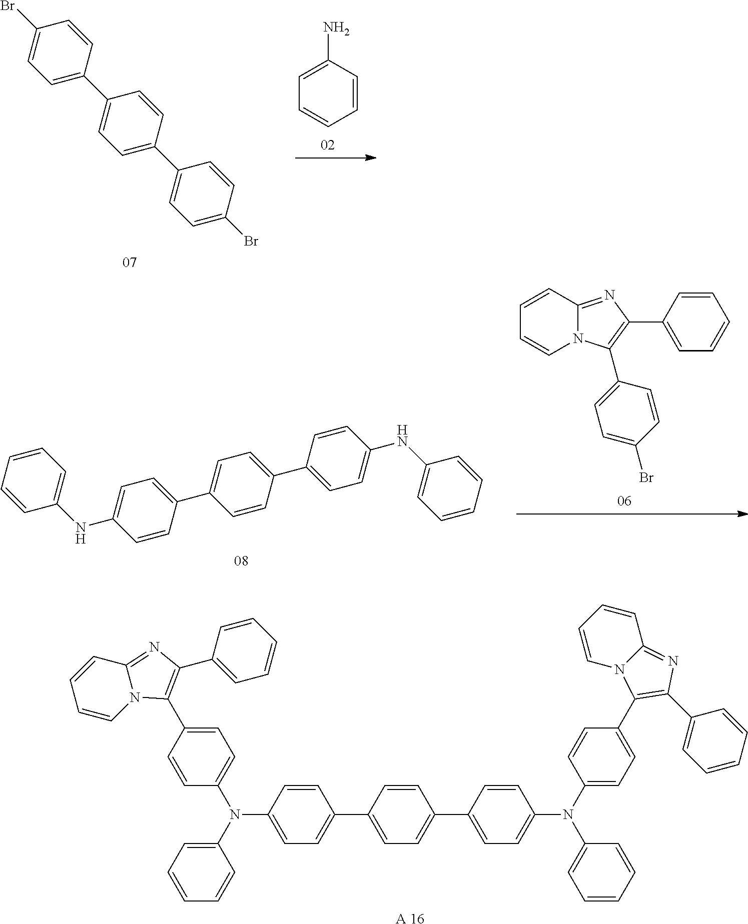 [SMILES: Brc1ccc(-c2ccc(-c3ccc(Br)cc3)cc2)cc1 ; Nc1ccccc1 ; c1ccc(Nc2ccc(-c3ccc(-c4ccc(Nc5ccccc5)cc4)cc3)cc2)cc1 ; Brc1ccc(-c2c(-c3ccccc3)nc3ccccn23)cc1 ; c1ccc(-c2nc3ccccn3c2-c2ccc(N(c3ccccc3)c3ccc(-c4ccc(-c5ccc(N(c6ccccc6)c6ccc(-c7c(-c8ccccc8)nc8ccccn78)cc6)cc5)cc4)cc3)cc2)cc1 ; "A 16"]

Synthesis of the compound 08: the compound was synthesized by selecting the corresponding materials, and by reference to the synthesis way and treatment method of the compound 03 via changing the corresponding raw materials only. Mass spectrometry: 412.5 (M+H)

Synthesis of the compound A16: a yellow solid compound A16 was obtained by selecting the corresponding materials, and by reference to the synthesis and sublimation of the compound A1. Mass spectrometry: 949.1 (M+H). ¹H NMR (400 MHz, CDCl₃) δ 8.48 (d, 2H), 7.73 (dd, 4H), 7.58-7.44 (m, 12H), 7.37 (m, 8H), 7.33-7.17 (m, 14H), 7.08 (d, 4H), 7.00 (d, 2H), 6.86 (d, 2H).

### (3) Synthesis of the Compound A17

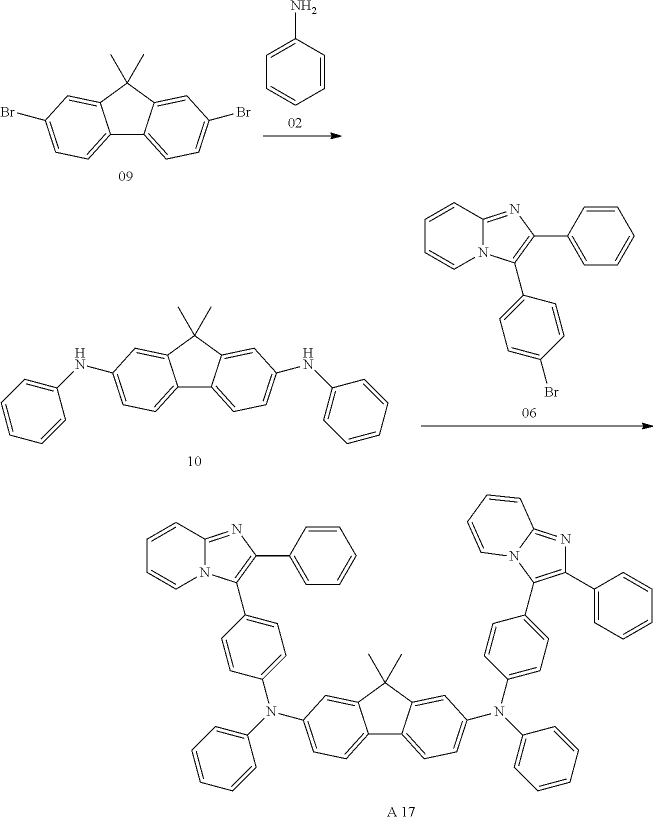

Synthesis of the compound 10: the compound was synthesized by selecting the corresponding materials, and by reference to the synthesis way and treatment method of the compound 03 via changing the corresponding raw materials only. Mass spectrometry: 377.5 (M+H)

Synthesis of the compound A17: a yellow solid compound A17 was obtained by selecting the corresponding materials, and by reference to the synthesis and sublimation of the compound A1. Mass spectrometry: 913.1 (M+H). ¹HNMR (400 MHz, CDCl₃) δ 8.48 (d, 2H), 7.86 (d, 2H), 7.73 (dd, 4H), 7.49 (dd, J=14.4, 9.4 Hz, 10H), 7.37 (m, 4H), 7.25 (dd, J=28.1, 8.1 Hz, 12H), 7.08 (m, 4H), 7.00 (d, 2H), 6.86 (d, 2H), 1.69 (s, 6H).

### (4) Synthesis of the Compound A22

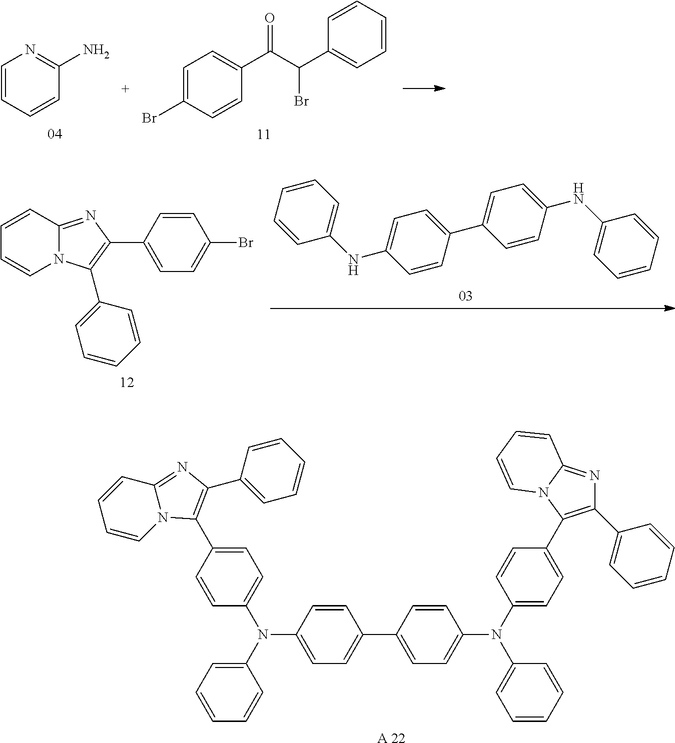

Synthesis of the compound 12: the compound was synthesized by selecting the corresponding materials, and by reference to the synthesis way and treatment method of the compound 06 via changing the corresponding raw materials only. Mass spectrometry: 349.03 (M+H)

Synthesis of the compound A22: a yellow solid compound A22 was obtained by selecting the corresponding materials, and by reference to the synthesis and sublimation of the compound A1. Mass spectrometry: 873.1 (M+H). ¹HNMR (400 MHz, CDCl₃) δ 8.48 (d, 2H), 7.73 (dd, 4H), 7.60-7.42 (m, 12H), 7.37 (m, 8H), 7.33-7.16 (m, 10H), 7.04 (d, J=40.0 Hz, 6H), 6.86 (d, 2H).

### (5) Synthesis of the Compound B86

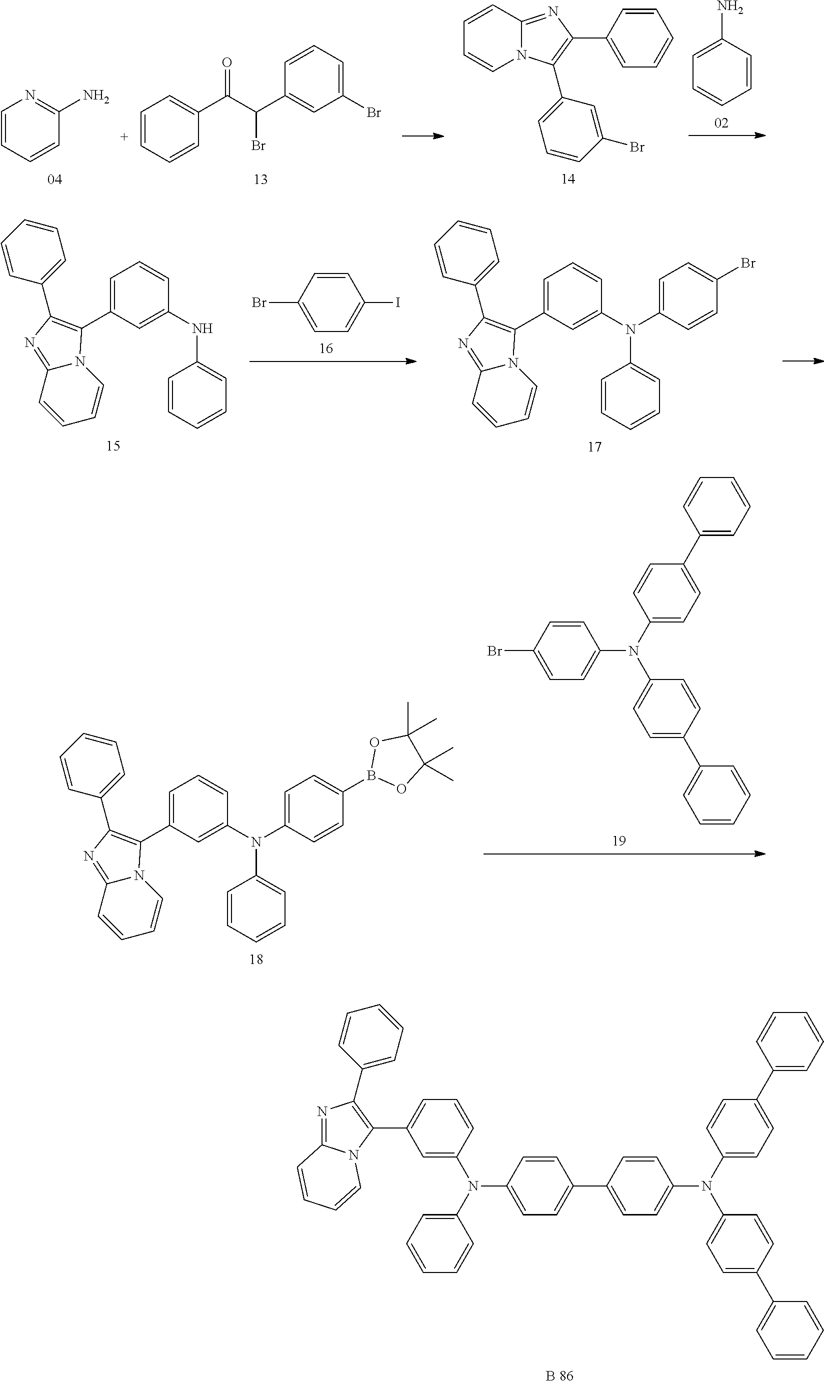

Synthesis of the compound 14: the compound was synthesized by selecting the corresponding materials, and by reference to the synthesis way and treatment method of the compound 06 via changing the corresponding raw materials only. Mass spectrometry: 397.30 (M+H);

Synthesis of the compound 15: the compound was synthesized by selecting the corresponding materials, and by reference to the synthesis way and treatment method of the compound 03 via changing the corresponding raw materials only. Mass spectrometry: 361.4 (M+H);

Synthesis of the compound 17: the compound 15 (45 g, 124.5 mmol, 1.0 eq), compound 16 (36.98 g, 130.7 mmol, 1.05 eq), CuI (2.37 g, 12.45 mmol, 2 eq), 1,10-phenanthroline (4.49 g, 24.9 mmol, 0.2 eq), K₂CO₃ (34.41 g, 2.49 mmol, 0.04 eq), DMF (450 ml) were successively fed into a 2 L three-necked flask, vacuumized and replaced with nitrogen for 3 times, then heated up to 120° C. around in an oil bath and stirred for 8 h under heat preservation; sampling was performed, and completion of the reaction of the raw material 15 was monitored by TLC. The reaction liquid was cooled to 40° C., and dropwisely added deionized water (800 ml) and stirred for 1 h, then cooled to room temperature and filtered; the obtained solid was added toluene (400 ml) to be dissolved fully, then washed with deionized water (100 ml*3) with deionized water; after liquid separation, organic phases were filtered with silica gel; the silica gel was washed with a small amount of toluene, then the organic phases were concentrated to be remaining 250 ml; methanol (300 ml) was then added dropwisely for crystallization, then filtering and drying were performed to obtain 50.34 g of a creamy white solid compound 17 with a yield of 78.3%. Mass spectrometry: 516.4 (M+H)

Synthesis of the compound 18: the compound 17 (32.5 g, 62.9 mmol, 1.0 eq), bis(pinacolato)diboron (19.18 g, 75.52 mmol, 1.2 eq), Pd(dppf)Cl₂ (0.92 g, 1.26 mmol, 0.02 eq), potassium acetate (12.35 g, 125.8 mmol, 2 eq) and dioxane (350 ml) were successively fed into a 1 L single-necked flask, then heated up to 100° C. around in an oil bath and stirred for 6 h under heat preservation; sampling was performed, and completion of the reaction of the raw material 17 was monitored by TLC. The reaction liquid was cooled to 40° C., and concentrated under reduced pressure to 200 ml, then added methanol (400 ml) and stirred for 2 h at room temperature, and filtered to obtain a solid; the obtained solid was added n-hexane (400 ml) for beating for 2 h at 50° C., filtered and dried to obtain 30.35 g of a creamy white solid compound 18 with a yield of 86.1%. Mass spectrometry: 563.5 (M+H)

Synthesis of the compound B86: the compound 18 (28.0 g, 49.69 mmol, 1.0 eq), compound 19 (23.67 g, 49.69 mmol, 1.0 eq), K₂CO₃ (13.73 g, 99.38 mmol, 2.0 eq), Pd₁₃₂ (0.35 g, 0.49 mmol, 0.01 eq), toluene (280 ml), ethanol (56 ml) and deionized water (56 ml) were successively fed into a 1 L three-necked flask, vacuumized and replaced with nitrogen for 3 times, then heated up to 75° C. around in an oil bath and stirred for 16 h at reflux under heat preservation; sampling was performed, and completion of the reaction of the raw material 18 was monitored by TLC. The reaction liquid was cooled to 60° C., and added with toluene (200 ml) and deionized water (100 ml), then stirred for 1 h, and subjected to liquid separation, afterwards, organic phases were filtered with silica gel, and the silica gel was washed with a small amount of toluene, then the organic phases were concentrated to obtain a solid; and the solid was recrystallized with toluene/methanol (220 ml/250 ml) for three times, and dried to obtain 29.47 g of a light yellow solid with a yield of 71.2%. The obtained composite was sublimated and purified to obtain 22.3 g of a light yellow solid compound B86 with a yield of 75.6%. Mass spectrometry: 833.1 (M+H). ¹H NMR (400 MHz, CDCl₃) δ 8.48 (d, 1H), 7.75 (dd, 4H), 7.62-7.42 (m, 19H), 7.39 (m, J=20.0 Hz, 9H), 7.33-7.14 (m, 7H), 7.08 (d, 2H), 7.00 (d, 1H), 6.86 (d, 1H).

### (6) Synthesis of the Compound B111

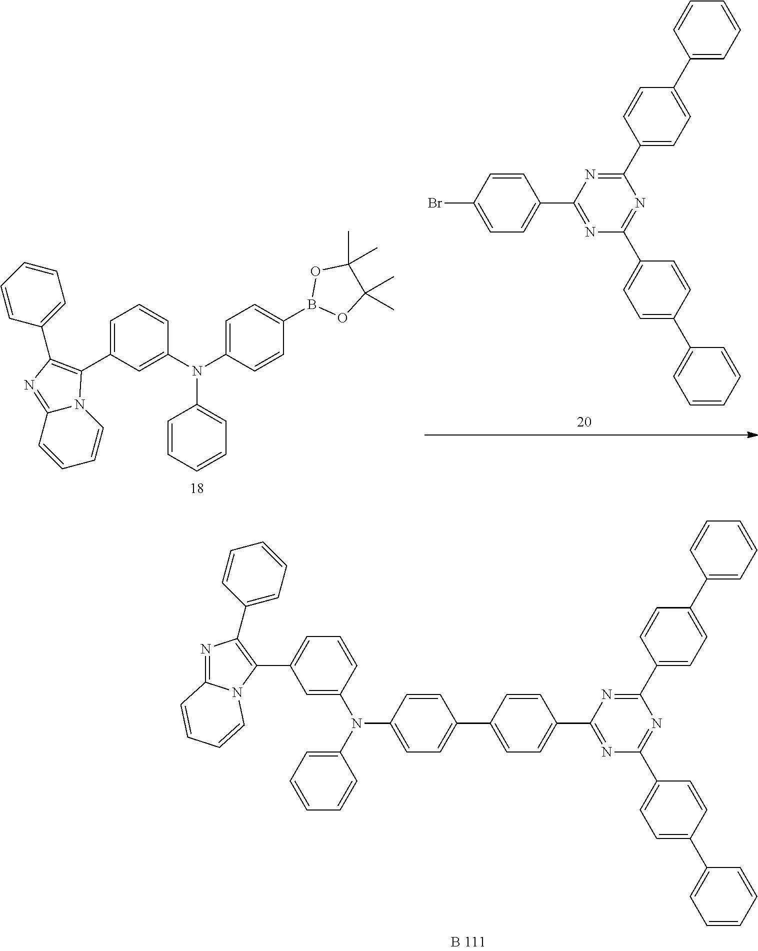 [SMILES: CC1(C)OB(c2ccc(N(c3ccccc3)c3cccc(-c4c(-c5ccccc5)nc5ccccn45)c3)cc2)OC1(C)C ; Brc1ccc(-c2nc(-c3ccc(-c4ccccc4)cc3)nc(-c3ccc(-c4ccccc4)cc3)n2)cc1 ; c1ccc(-c2ccc(-c3nc(-c4ccc(-c5ccccc5)cc4)nc(-c4ccc(-c5ccc(N(c6ccccc6)c6cccc(-c7c(-c8ccccc8)nc8ccccn78)c6)cc5)cc4)n3)cc2)cc1]

Synthesis of the compound B111: the compound was synthesized by selecting the corresponding materials, and by reference to the synthesis way and treatment method of the compound B86 via changing the corresponding raw materials only. Mass spectrometry: 897.1 (M+H). ¹H NMR (400 MHz, CDCl₃) δ 8.48 (d, 1H), 7.96 (m, 6H), 7.75 (m, 4H), 7.60-7.34 (m, 16H), 7.33-7.15 (m, 13H), 7.08 (d, 2H), 7.00 (d, 1H), 6.86 (d, 1H).

### (7) Synthesis of the Compound B130

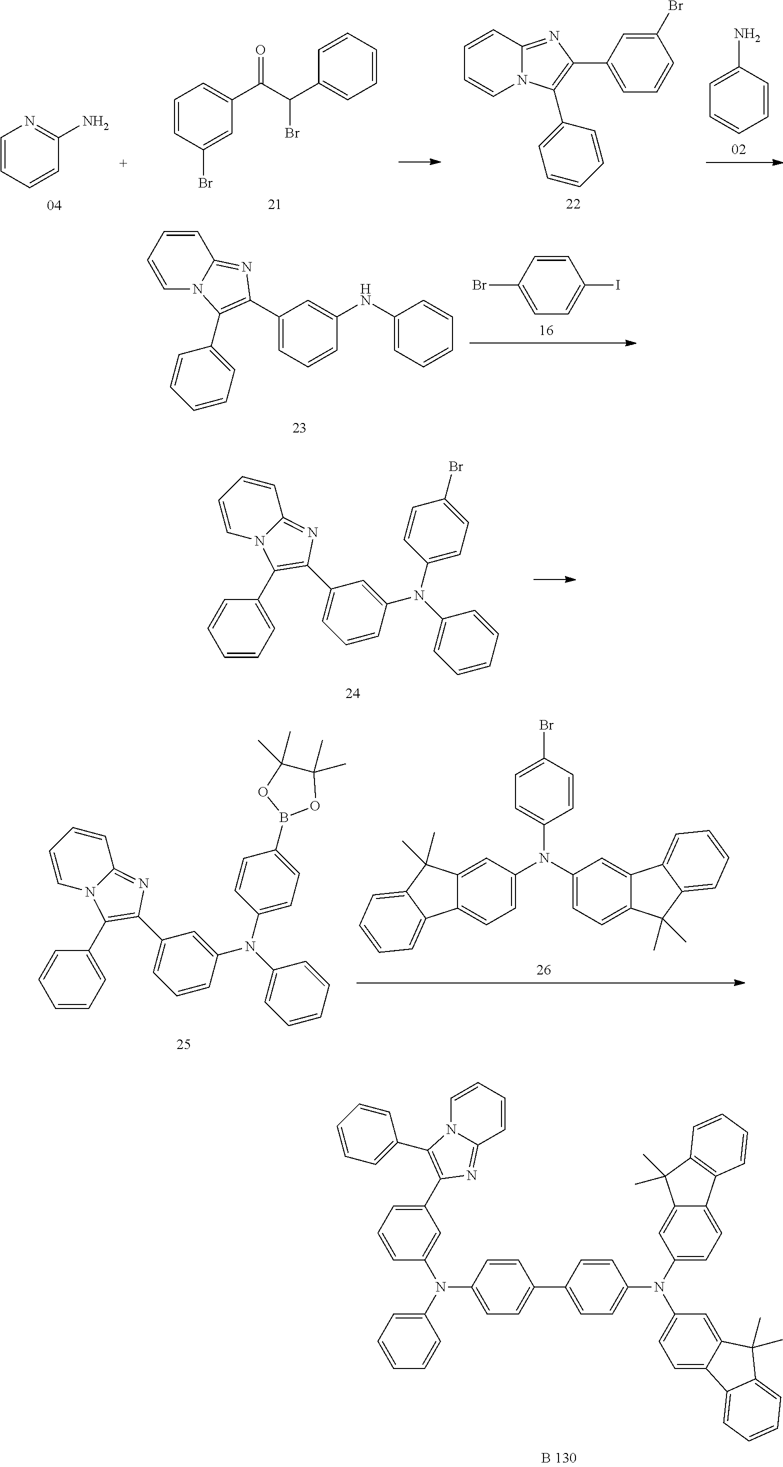

Synthesis of the compound B130: the compound was synthesized by selecting the corresponding materials, and by reference to the synthesis way and treatment method of the compound B86 via changing the corresponding raw materials only. Mass spectrometry: 913.2 (M+H). ¹H NMR (400 MHz, CDCl₃) δ 8.48 (d, 1H), 7.95-7.79 (m, 6H), 7.60-7.44 (m, 14H), 7.36 (m, J=13.6 Hz, 7H), 7.30-7.15 (m, 8H), 7.08 (d, 2H), 7.00 (d, 1H), 6.86 (d, 1H), 1.69 (s, 12H).

### (8) Synthesis of the Compound B137

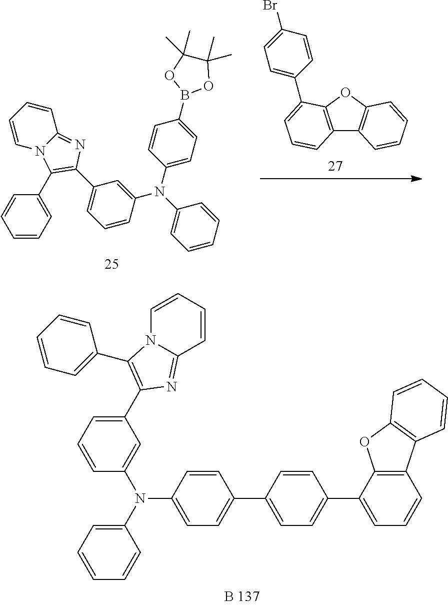

Synthesis of the compound B137: the compound was synthesized by selecting the corresponding materials, and by reference to the synthesis way and treatment method of the compound B86 via changing the corresponding raw materials only. Mass spectrometry: 679.8 (M+H). ¹HNMR (400 MHz, CDCl₃) 8.48 (d, 1H), 8.13-7.94 (m, 3H), 7.84 (d, 2H), 7.63-7.46 (m, 9H), 7.45-7.29 (m, 5H), 7.29-7.13 (m, 9H), 7.04 (d, J=40.0 Hz, 3H), 6.86 (d, 1H).

### Application Example

(1) Comparison of the compound performance: the compound of the present invention may serve as a light extraction layer material in an OLED device, and has a higher glass-transition temperature, a higher refractive index and a small refractive index difference in the visible light region. Basic performances are listed in Table 1 below

TABLE 1
Refractive index comparison

| | Glass-transition | Sublimation | Refractive index | | | | | |
|---|---|---|---|---|---|---|---|---|
| | temperature | temperature | @450 | @520 | @630 | Refractive index difference | | |
| | (° C.) | (° C.) | nm | nm | nm | ΔB-G | ΔB-R | ΔG-R |
| Compound A1 | 150 | 370 | 2.09 | 2.03 | 1.97 | 0.03 | 0.12 | 0.06 |
| Compound A16 | 152 | 378 | 2.04 | 1.99 | 1.95 | 0.06 | 0.09 | 0.04 |
| Compound A17 | 141 | 365 | 2.11 | 2.06 | 1.99 | 0.05 | 0.12 | 0.07 |
| Compound A22 | 134 | 365 | 2.10 | 2.01 | 1.97 | 0.09 | 0.13 | 0.06 |
| Compound B86 | 149 | 370 | 2.15 | 2.07 | 2.03 | 0.08 | 0.12 | 0.04 |
| Compound B111 | 154 | 380 | 2.05 | 1.98 | 1.95 | 0.07 | 0.10 | 0.04 |
| Compound B130 | 152 | 378 | 2.15 | 2.09 | 2.04 | 0.06 | 0.11 | 0.05 |
| Compound B137 | 124 | 363 | 2.07 | 1.99 | 1.96 | 0.08 | 0.11 | 0.03 |
| HTM1 | 134 | 385 | 1.99 | 1.91 | 1.85 | 0.08 | 0.14 | 0.06 |

(2) Manufacture of an Organic Light-Emitting Device

50 mm*50 mm*1.0 mm glass substrate having ITO (100 nm) transparent electrodes was subjected to ultrasonic cleaning for 10 min in ethanol, and dried at 150° C., then treated by N2 Plasma for 30 min. The washed glass substrate was mounted on a substrate support of a vacuum evaporation device; a compound HATCN was evaporated on a face with transparent electrode wires first by covering transparent electrodes to form a thin film having a film thickness of 5 nm; a layer of HTM1 was then evaporated to form a thin film having a film thickness of 60 nm, and a layer of HTM2 was evaporated on the HTM1 film to form a thin film having a film thickness of 10 nm, and then a host material CBP and a doping material were evaporated on the HTM2 film in a co-evaporation mode with a film thickness of 30 nm; and a ratio of the host material to the doping material was 90%:10%. BCP (5 nm) and Alq₃ (30 nm) were respectively evaporated on a light-emitting layer as a hole blocking layer material and an electron transport material successively according to the allocation of the table below, and then LiF (1 nm) was evaporated on the electron transport material layer as an electron injection material; afterwards, Mg/Ag (18 nm, 1:9) was evaporated as a cathode material in a co-evaporation mode, and finally, CPL (50 nm) was evaporated on the cathode material as a light extraction layer material according to the allocation of the table below.

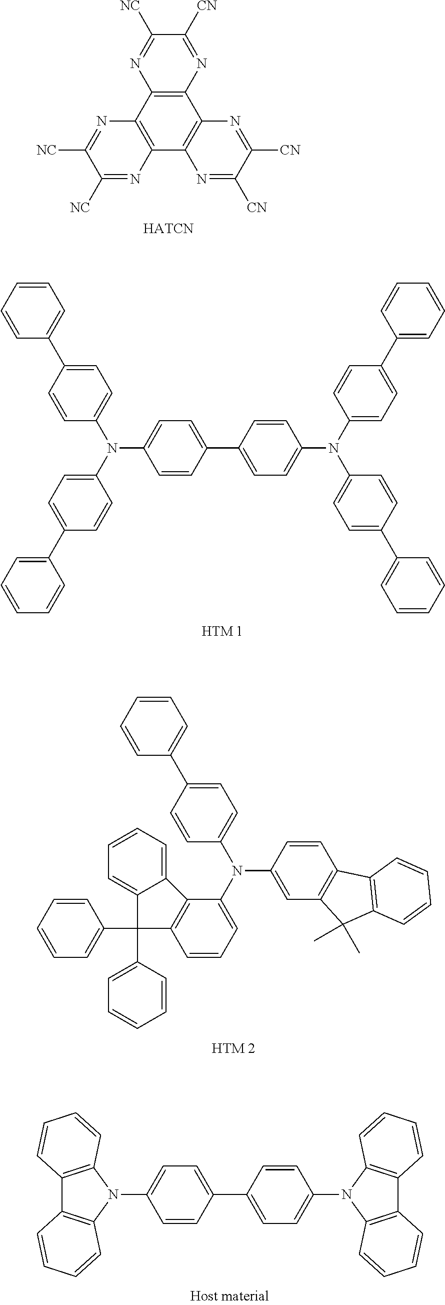

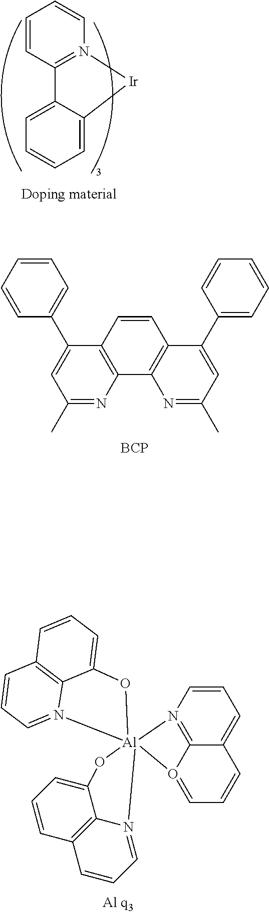

Evaluation on the Device Performance

The above device was subjected to device performance test, in each example and comparative example, a constant current supply (Keithley 2400) was used to flow through a light-emitting element with a constant electric current density; a spectroradiometer (CS 2000) was used to test light emission spectrum, and to test the luminous efficiency of the device. Results are as shown in Table 2 below:

TABLE 2

| | | Current |
|---|---|---|
| | | efficiency |
| | | (Cd/A) |
| Device | CPL | @ 3000nits |
| Example 1 | Compound A1 | 52 |
| Example 2 | Compound A16 | 52 |
| Example 3 | Compound A17 | 53 |
| Example 4 | Compound A22 | 51 |
| Example 5 | Compound B86 | 50 |
| Example 6 | Compound B111 | 49 |
| Example 7 | Compound B130 | 52 |
| Example 8 | Compound B137 | 51 |
| Comparative | HTM1 | 48 |
| Example 1 | | |

It can be seen from data of the above Table 2 that compared with comparative compounds, the compound of the present invention is applied to the light extraction layer material of the organic light-emitting device to show more excellent luminous efficiency.

As mentioned above, the imidazopyridine-based arylamine compound containing the structure of the present invention has the advantages such as, a low sublimation temperature, a good thermal stability, a high refractive index, and a small refractive index difference in the visible light region, and can substantially improve the light extraction efficiency and film state stability. The OLED device prepared by the series of compounds can achieve a higher efficiency and improved durability. To sum up, such kind of compound can serve as a light extraction layer material and has a possibility of being applied to AMOLED industry.

1. An imidazopyridine-based arylamine compound, having a structural formula as shown in Formula I: 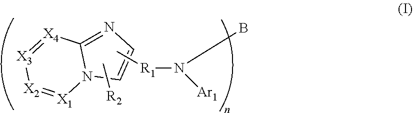
wherein n is 1 or 2;
X1, X2, X3 and X4 independently represent CR₀ or N; R₀ is independently selected from hydrogen, deuterium, halogen, C1-C8 alkyl, C1-C8 heteroalkyl, aralkyl, amino, silicyl, substituted or unsubstituted C6-C60 aryl, substituted or unsubstituted C1-C60 heteroaryl, nitrile, isonitrile; and adjacent R₀ are capable of being bonded to form a fused ring;
R₁ is a single bond, C1-C30 alkylene, C1-C30 heteroalkylene, C3-C30 cycloalkylene, substituted or unsubstituted C6-C30 arylene, and substituted or unsubstituted C2-C28 heteroarylene;
R2 is independently selected from hydrogen, deuterium, halogen, C1-C30 alkyl, C1-C30 heteroalkyl, C3-C30 cycloalkyl, C1-C30 alkoxy, C6-C60 aryloxy, amino, silicyl, nitrile, isonitrile, phosphino, substituted or unsubstituted C6-C60 aryl, and substituted or unsubstituted C1-C60 heteroaryl;
Ar₁ is substituted or unsubstituted C6-C60 aryl, substituted or unsubstituted C6-C60 heteroaryl, C3-C60 cycloalkyl, and substituted or unsubstituted C6-C60 arylamino;
B is substituted or unsubstituted C6-C60 aryl or arylene, substituted or unsubstituted C6-C60 heteroaryl or heteroarylene, C3-C60 cycloalkyl, and substituted or unsubstituted C6-C60 arylamino or arylimino;
and wherein one or more carbon atoms in heteroalkyl or heteroaryl are replaced by at least one heteroatom selected from O, S, N, Se, Si and Ge; the substitution refers to a substitution by deuterium, halogen, C1-C30 alkyl, phenyl, naphthyl or biphenyl.

2. The compound according to claim 1, having a structural formula as shown in Formula (II): 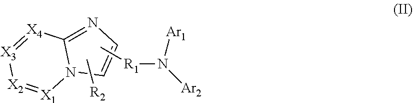
wherein R₁ is a single bond, C1-C10 alkylene, C1-C10 heteroalkylene, C3-C10 cycloalkylene, substituted or unsubstituted C6-C30 arylene, and substituted or unsubstituted C2-C28 heteroarylene;
R2 is independently selected from hydrogen, deuterium, halogen, C1-C10 alkyl, C1-C10 heteroalkyl, C1-C10 alkoxy, C3-C30 cycloalkyl, C6-C30 aryloxy, amino, silicyl, nitrile, isonitrile, phosphino, substituted or unsubstituted C6-C30 aryl, and substituted or unsubstituted C1-C30 heteroaryl;
Ar₁ and Ar₂ are substituted or unsubstituted C6-C30 aryl, substituted or unsubstituted C6-C30 heteroaryl, substituted or unsubstituted monocyclic or polycyclic C3-C30 aliphatic ring or aromatic ring, and substituted or unsubstituted C6-C30 arylamino;
and wherein one or more carbon atoms in heteroalkyl or heteroaryl are replaced by at least one heteroatom selected from O, S, N, and Si; the substitution refers to a substitution by deuterium, halogen, C1-C8 alkyl, phenyl, naphthyl or biphenyl.

3. The compound according to claim 2, wherein R₁ is C1-C8 alkyl substituted or unsubstituted C6-C20 arylene, C1-C8 alkyl substituted or unsubstituted C2-C18 heteroarylene; R₂ is C1-C8 alkyl, C1-C8 alkyl substituted or unsubstituted C6-C20 aryl, C1-C8 alkyl substituted or unsubstituted C1-C20 heteroaryl; Ar₁ and Ar₂ are substituted or unsubstituted C6-C20 aryl, substituted or unsubstituted C6-C20 heteroaryl, substituted or unsubstituted monocyclic or polycyclic C3-C20 aliphatic ring or aromatic ring, substituted or unsubstituted C6-C20 arylamino; wherein one or more carbon atoms in heteroalkyl or heteroaryl are replaced by at least one heteroatom selected from O, S and N; the substitution refers to a substitution by deuterium, C1-C8 alkyl, phenyl, naphthyl or biphenyl.

4. The compound according to claim 3, wherein R₁ is C1-C4 alkyl substituted or unsubstituted C6-C10 arylene, C1-C4 alkyl substituted or unsubstituted C2-C8 heteroarylene; Ar₁ and R₂ are C1-C4 alkyl substituted or unsubstituted C6-C10 aryl, C1-C4 alkyl substituted or unsubstituted C1-C8 heteroaryl; Ar₂ is substituted or unsubstituted C6-C20 aryl, substituted or unsubstituted C6-C20 heteroaryl, substituted or unsubstituted monocyclic or polycyclic C3-C20 aliphatic ring or aromatic ring, substituted or unsubstituted C6-C20 acylamino; wherein one or more carbon atoms in heteroalkyl or heteroaryl are replaced by at least one heteroatom selected from 0, S and N; and the substitution refers to a substitution by deuterium, C1-C4 alkyl, phenyl, naphthyl or biphenyl.

5. The compound according to claim 2, wherein at least one of Ar₁ or Ar₂ has the following structural formula (III), 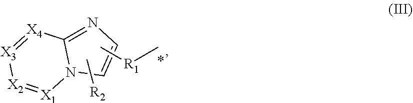
wherein R₁ is a single bond, C1-C8 alkylene, C1-C8 heteroalkylene, C3-C8 cycloalkylene, C1-C8 alkyl substituted or unsubstituted C6-C30 arylene, C1-C8 alkyl substituted or unsubstituted C2-C28 heteroarylene; wherein R₂ is H, deuterium, halogen, C1-C8 alkyl, C1-C8 heteroalkyl, C3-C8 cycloalkyl, C1-C8 alkyl substituted or unsubstituted C6-C30 aryl, and C1-C8 alkyl substituted or unsubstituted C1-C30 heteroaryl.

6. The compound according to claim 1, having a structural formula as shown in Formula (IV): 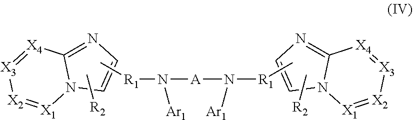
wherein R₁ is a single bond, C1-C8 alkylene, C1-C8 heteroalkylene, C3-C8 cycloalkylene, C1-C8 alkyl substituted or unsubstituted C6-C30 arylene, C1-C8 alkyl substituted or unsubstituted C2-C28 heteroarylene; wherein R₂ is hydrogen, deuterium, halogen, C1-C8 alkyl, C1-C8 heteroalkyl, C3-C8 cycloalkyl, C1-C8 alkyl substituted or unsubstituted C6-C30 aryl, C1-C8 alkyl substituted or unsubstituted C1-C30 heteroaryl; Ar₁ is substituted or unsubstituted C6-C30 aryl, substituted or unsubstituted C6-C30 heteroaryl, substituted or unsubstituted monocyclic or polycyclic C3-C30 aliphatic ring or aromatic ring, and A is substituted or unsubstituted C6-C30 arylene, and substituted or unsubstituted C6-C30 heteroarylene; and wherein one or more carbon atoms in heteroalkyl or heteroaryl are replaced by at least one heteroatom selected from O, S, N and Si; the substitution refers to a substitution by deuterium, halogen, C1-C8 alkyl, phenyl, naphthyl or biphenyl.

7. The compound according to claim 6, wherein R₁ is C1-C8 alkyl substituted or unsubstituted C6-C20 arylene, C1-C8 alkyl substituted or unsubstituted C2-C18 heteroarylene; R₂ is C1-C8 alkyl, C1-C8 alkyl substituted or unsubstituted C6-C20 aryl, C1-C8 alkyl substituted or unsubstituted C1-C20 heteroaryl; Ar₁ is substituted or unsubstituted C6-C20 aryl, substituted or unsubstituted C6-C20 heteroaryl, substituted or unsubstituted monocyclic or polycyclic C3-C20 aliphatic ring or aromatic ring; A is substituted or unsubstituted C6-C20 arylene, and substituted or unsubstituted C6-C20 heteroarylene; wherein one or more carbon atoms in heteroalkyl or heteroaryl are replaced by at least one heteroatom selected from O, S and N; the substitution refers to a substitution by deuterium, C1-C8 alkyl, phenyl, naphthyl or biphenyl.

8. The compound according to any one of claims 1-7, wherein X1, X2, X3 and X4 independently represent CR₀; and R₀ is independently selected from hydrogen and C1-C8 alkyl.

9. The compound according to claim 1, having a structural formula as shown in the following formulas:

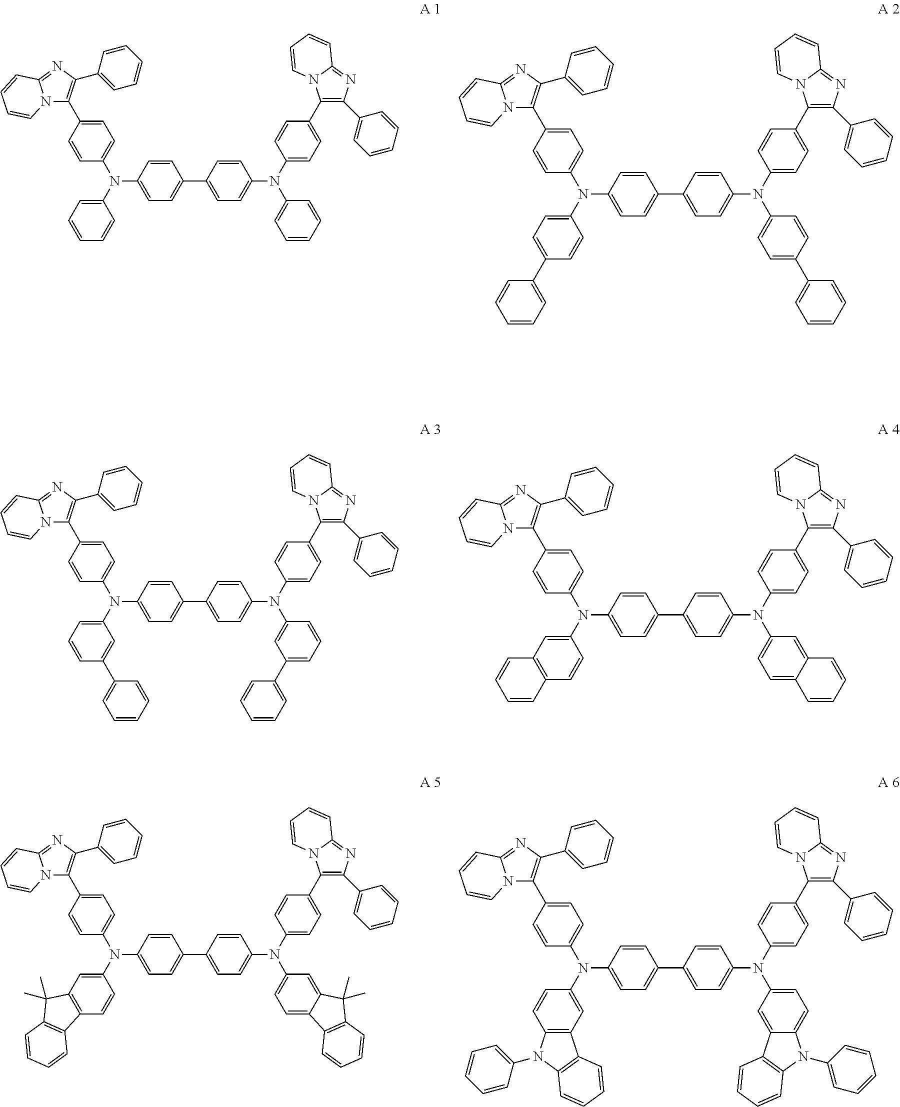

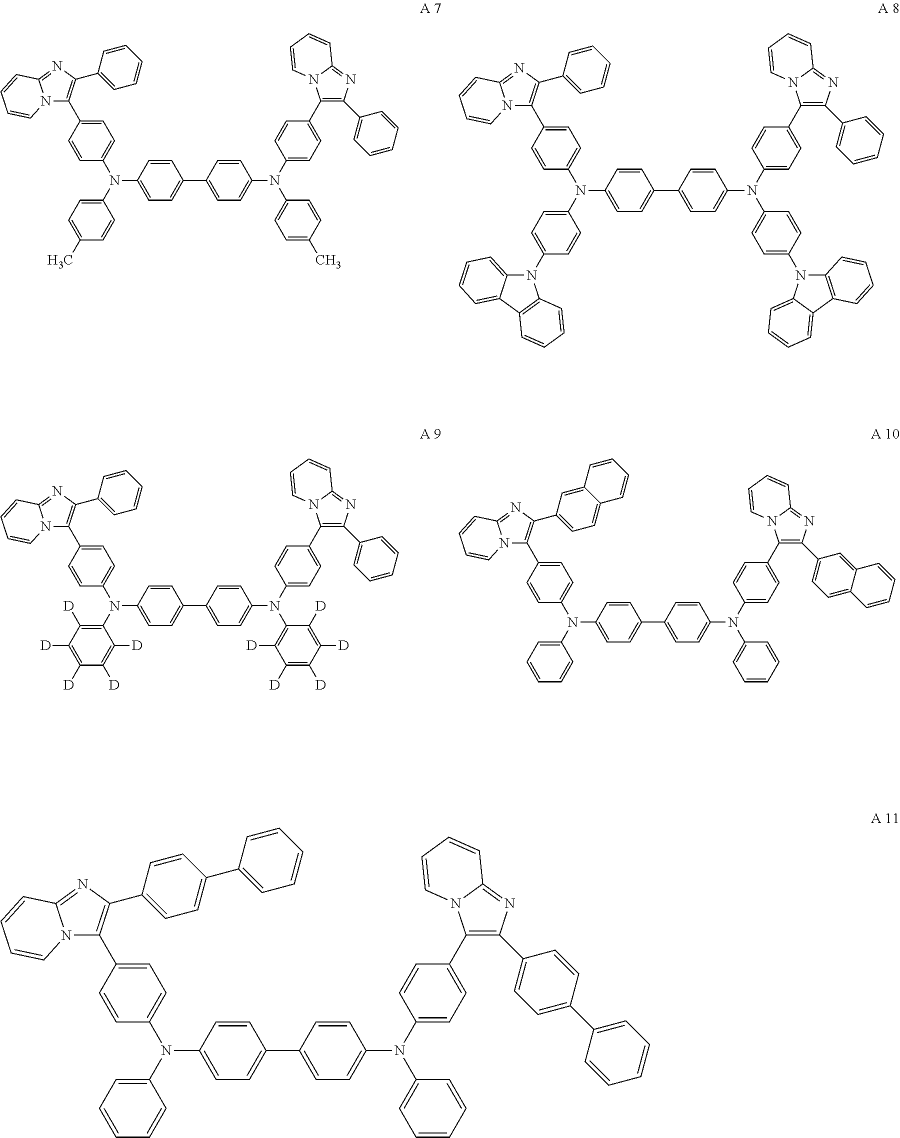

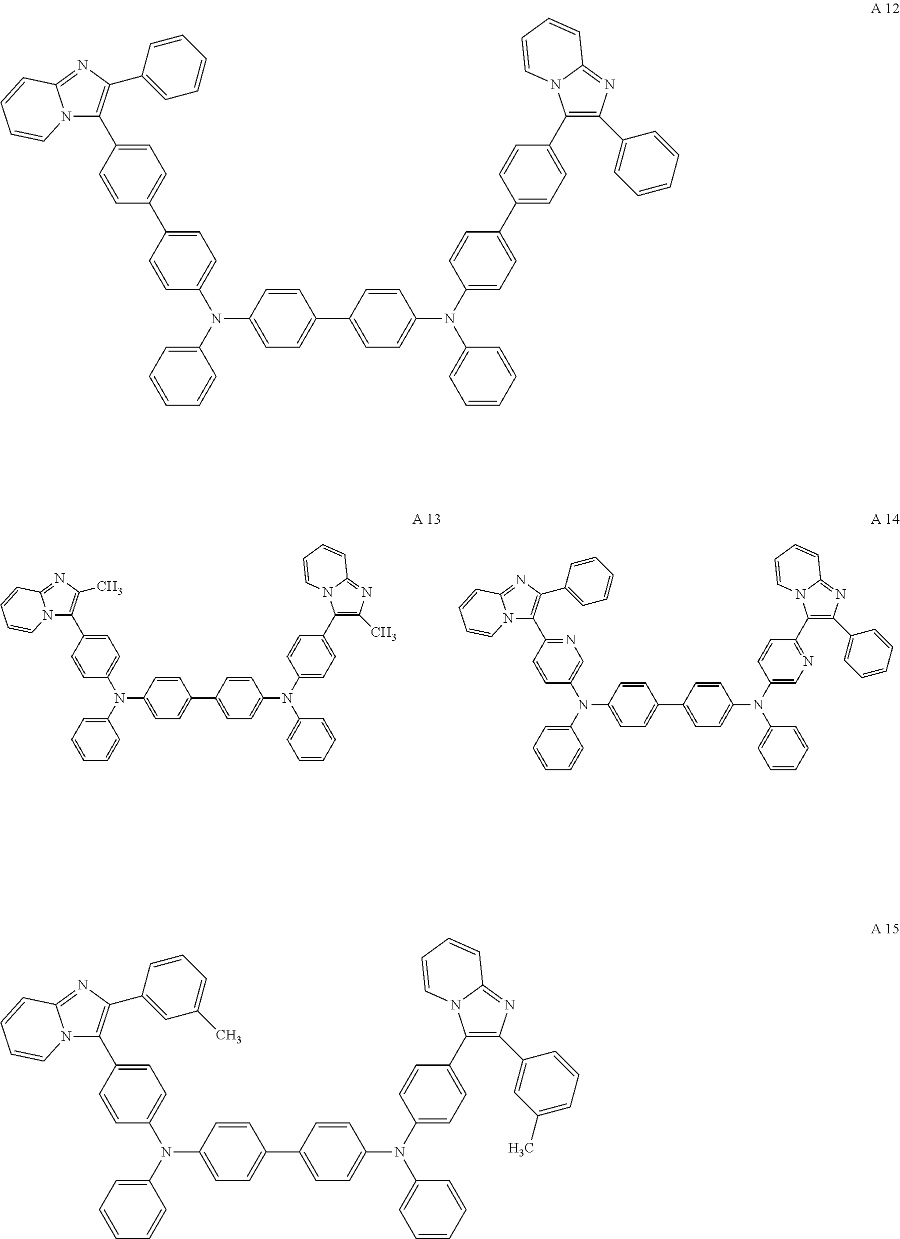 [SMILES: c1ccc(-c2nc3ccccn3c2-c2ccc(-c3ccc(N(c4ccccc4)c4ccc(-c5ccc(N(c6ccccc6)c6ccc(-c7ccc(-c8c(-c9ccccc9)nc9ccccn89)cc7)cc6)cc5)cc4)cc3)cc2)cc1 ; Cc1nc2ccccn2c1-c1ccc(N(c2ccccc2)c2ccc(-c3ccc(N(c4ccccc4)c4ccc(-c5c(C)nc6ccccn56)cc4)cc3)cc2)cc1 ; c1ccc(-c2nc3ccccn3c2-c2ccc(N(c3ccccc3)c3ccc(-c4ccc(N(c5ccccc5)c5ccc(-c6c(-c7ccccc7)nc7ccccn67)nc5)cc4)cc3)cn2)cc1 ; Cc1cccc(-c2nc3ccccn3c2-c2ccc(N(c3ccccc3)c3ccc(-c4ccc(N(c5ccccc5)c5ccc(-c6c(-c7cccc(C)c7)nc7ccccn67)cc5)cc4)cc3)cc2)c1]

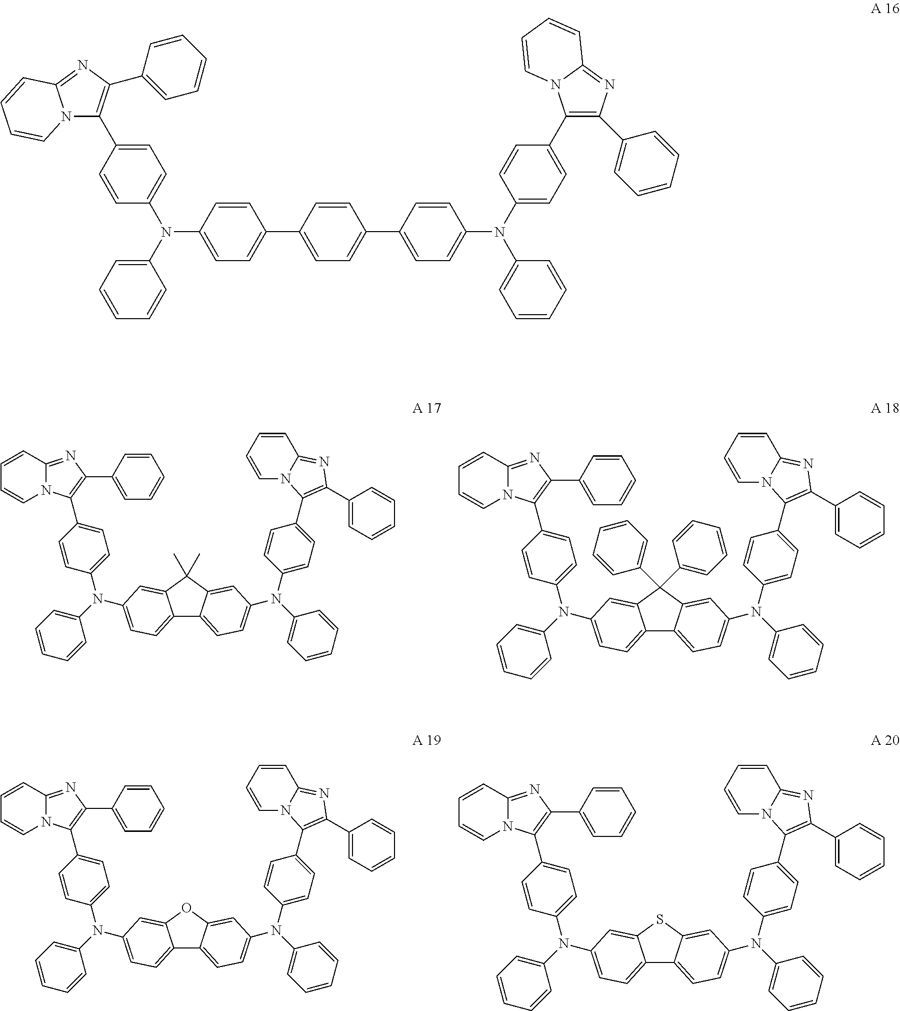

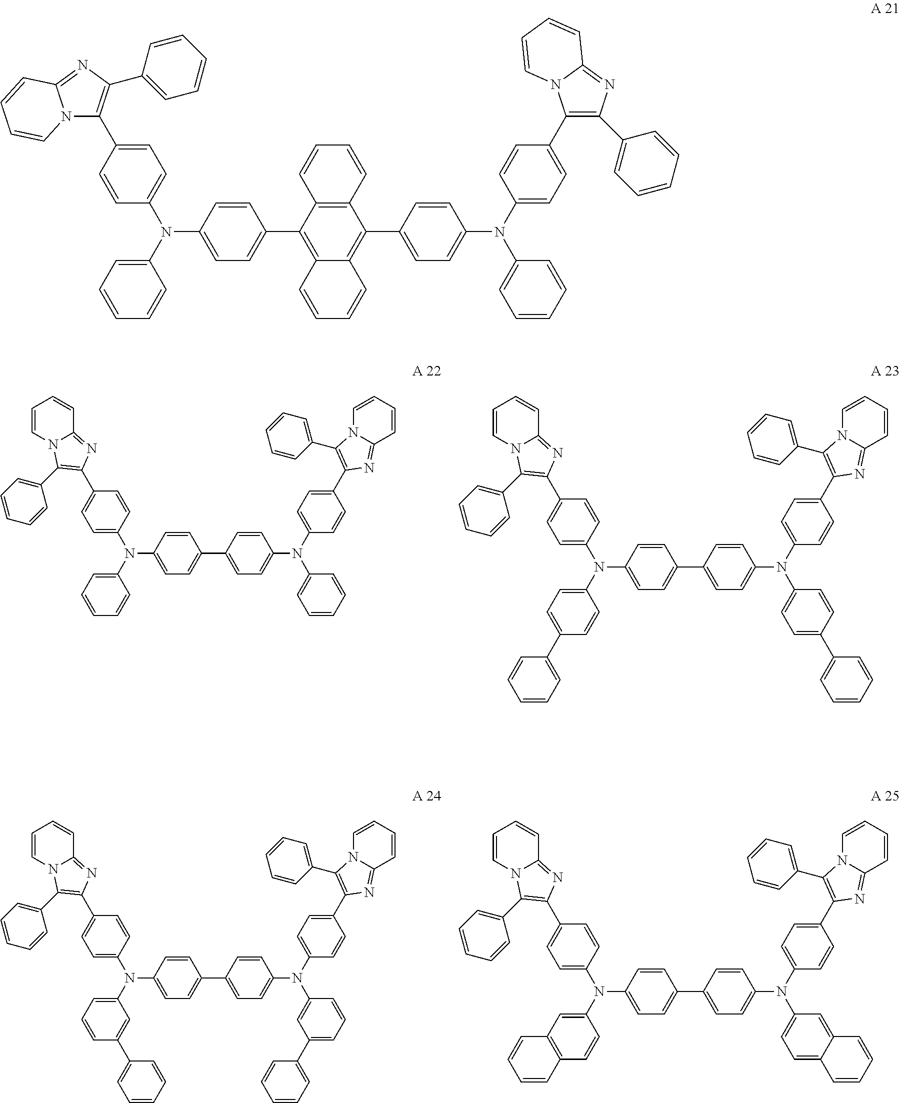

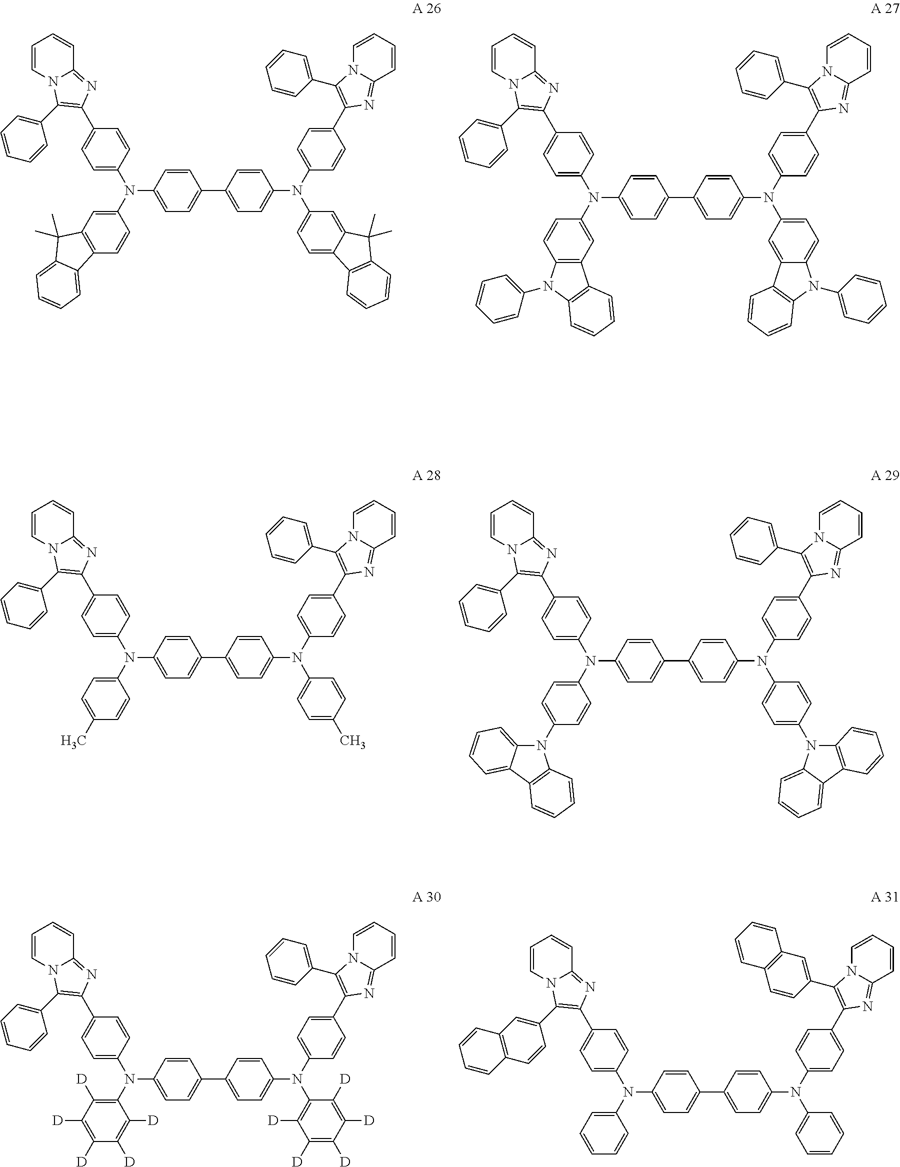

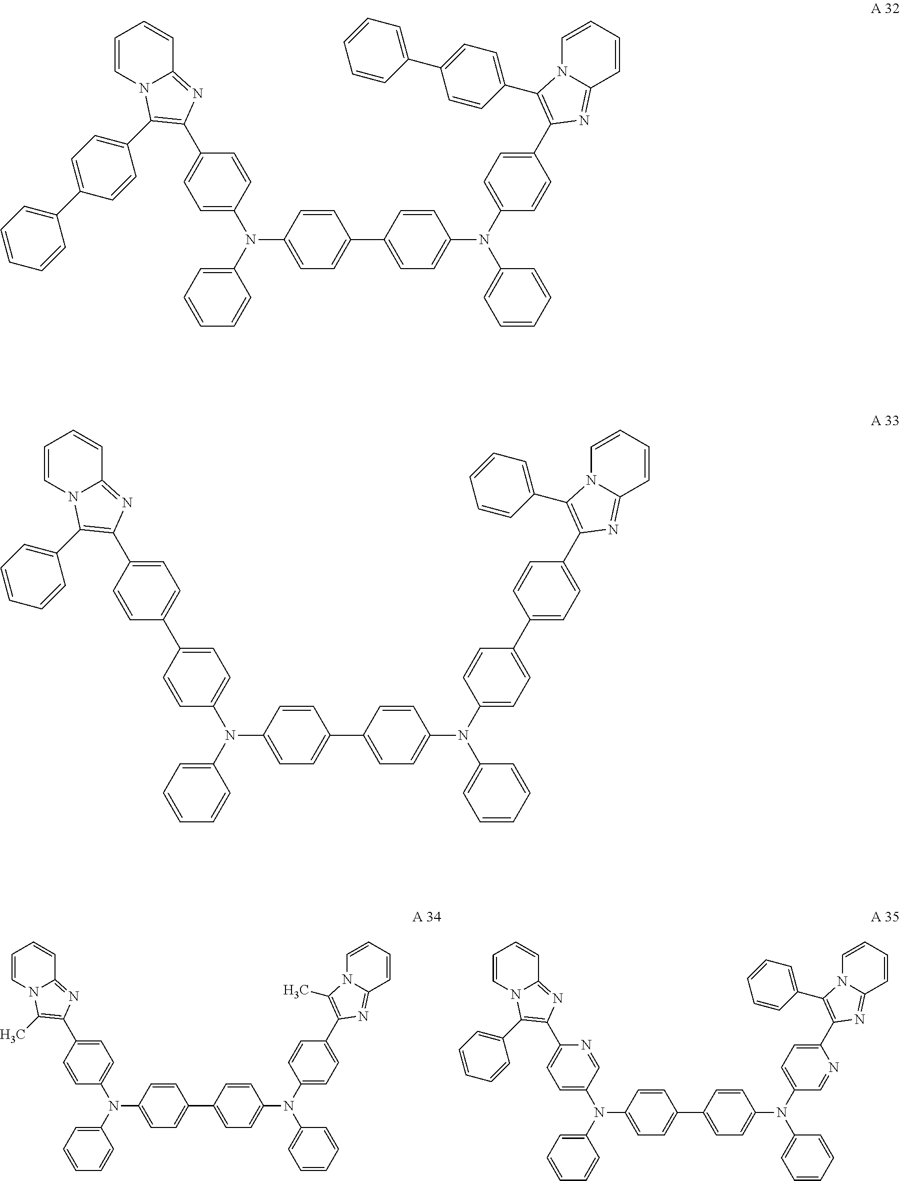 [SMILES: c1ccc(-c2ccc(-c3c(-c4ccc(N(c5ccccc5)c5ccc(-c6ccc(N(c7ccccc7)c7ccc(-c8nc9ccccn9c8-c8ccc(-c9ccccc9)cc8)cc7)cc6)cc5)cc4)nc4ccccn34)cc2)cc1 ; c1ccc(-c2c(-c3ccc(-c4ccc(N(c5ccccc5)c5ccc(-c6ccc(N(c7ccccc7)c7ccc(-c8ccc(-c9nc%10ccccn%10c9-c9ccccc9)cc8)cc7)cc6)cc5)cc4)cc3)nc3ccccn23)cc1 ; Cc1c(-c2ccc(N(c3ccccc3)c3ccc(-c4ccc(N(c5ccccc5)c5ccc(-c6nc7ccccn7c6C)cc5)cc4)cc3)cc2)nc2ccccn12 ; c1ccc(-c2c(-c3ccc(N(c4ccccc4)c4ccc(-c5ccc(N(c6ccccc6)c6ccc(-c7nc8ccccn8c7-c7ccccc7)nc6)cc5)cc4)cn3)nc3ccccn23)cc1]

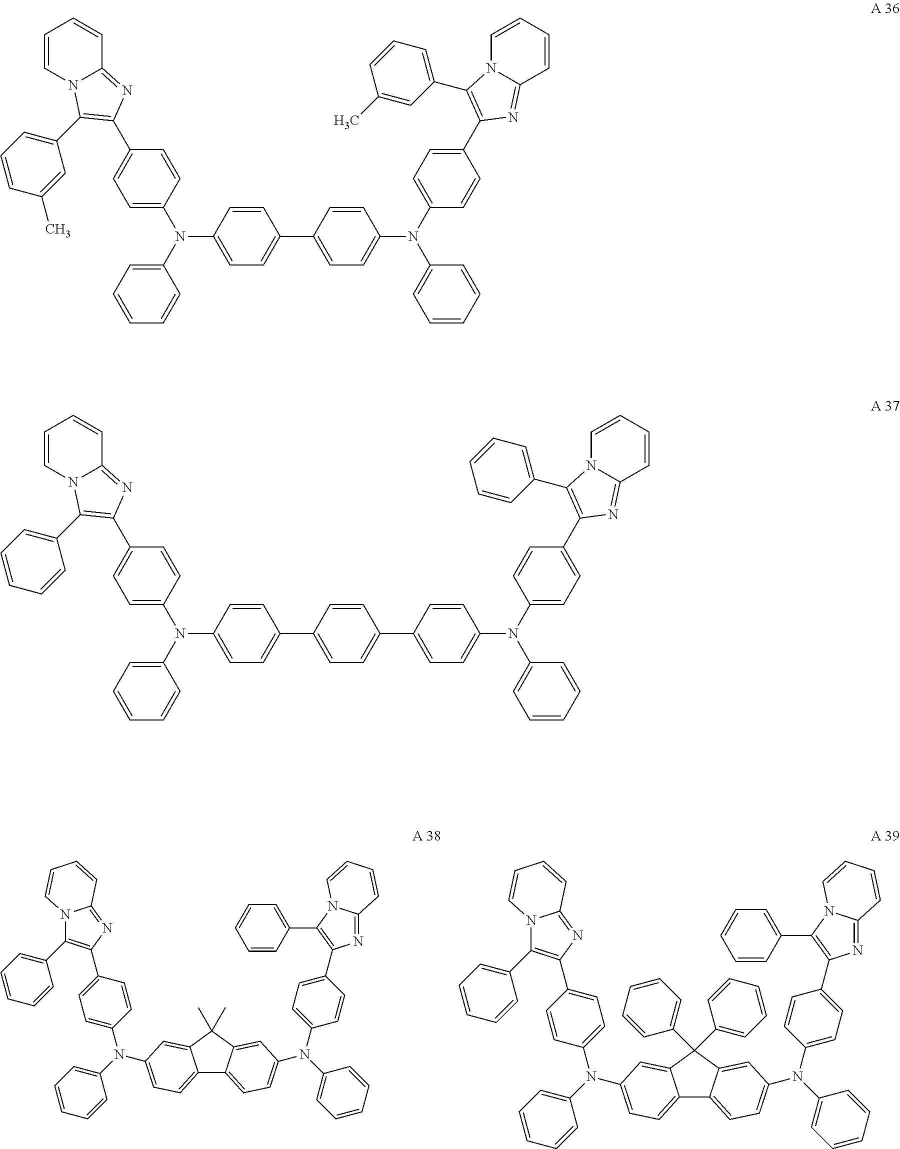

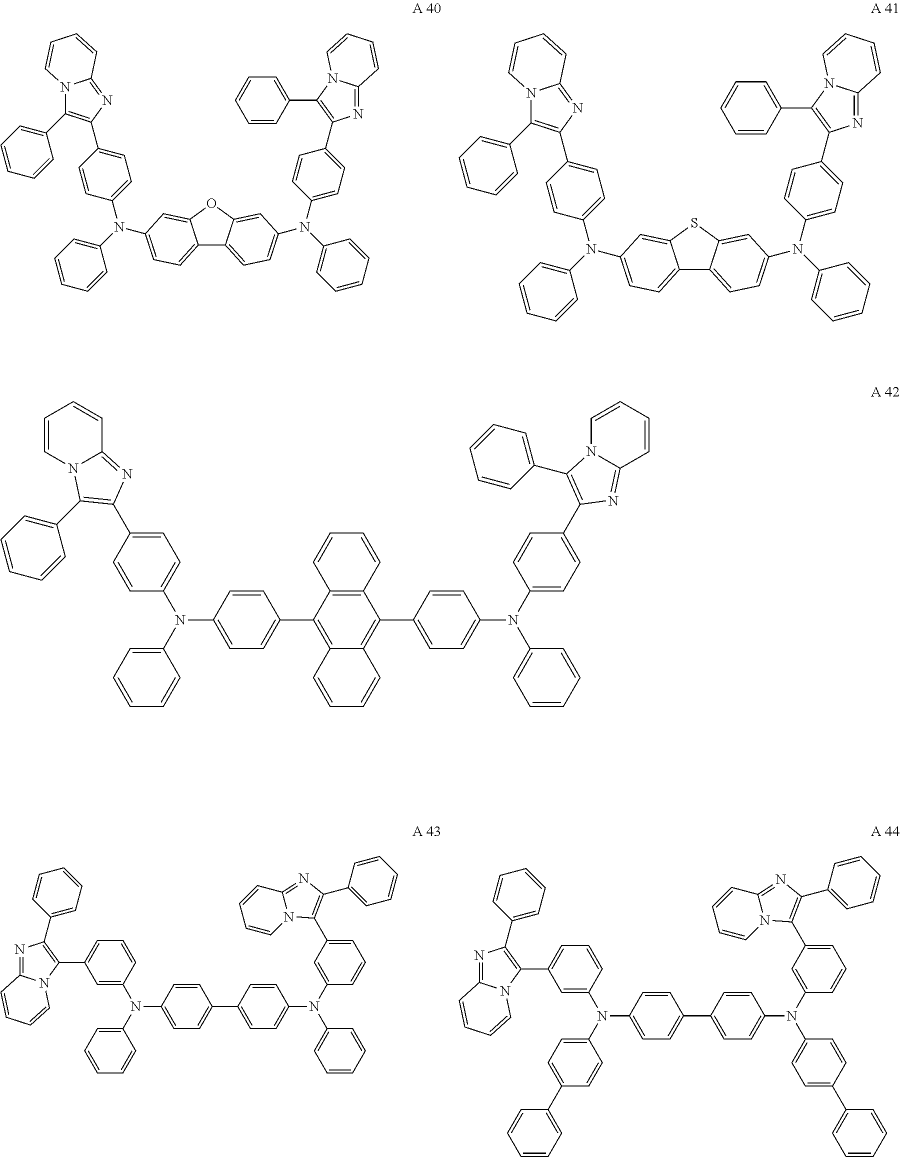

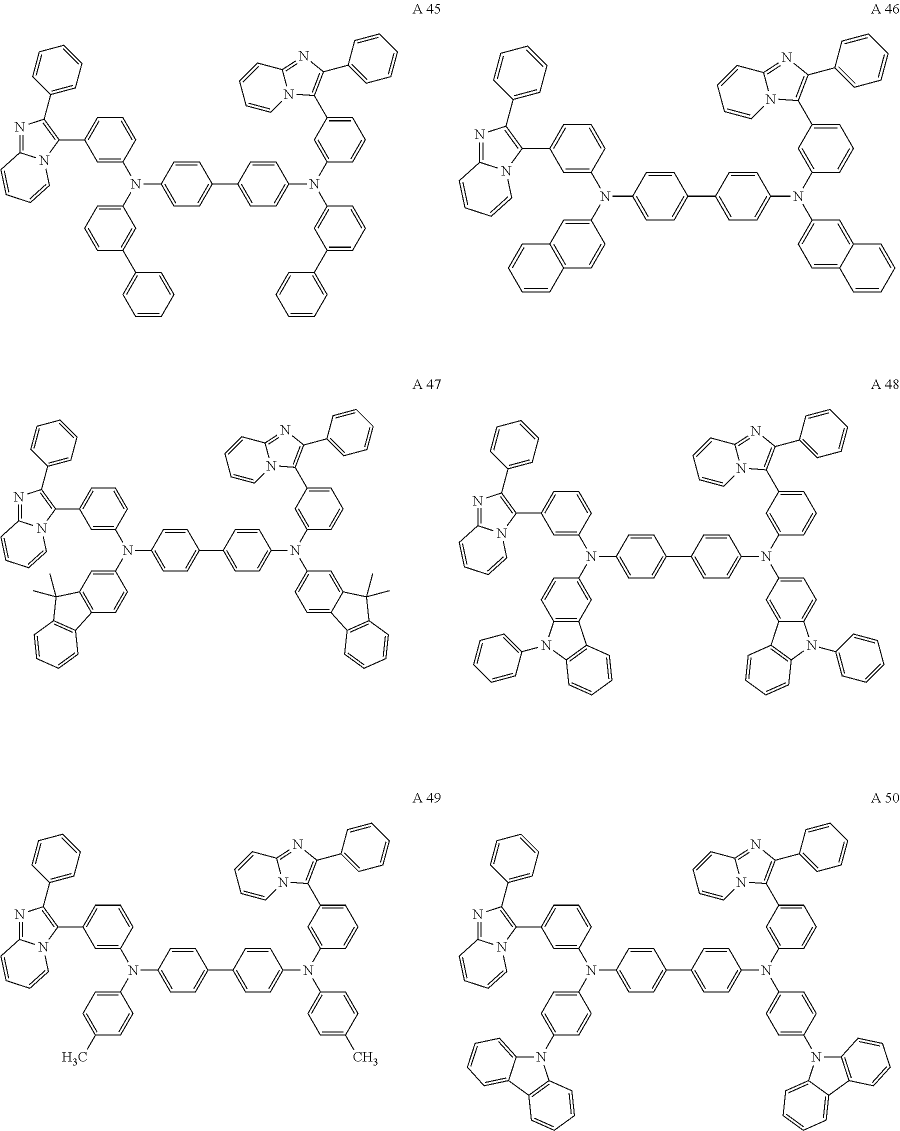

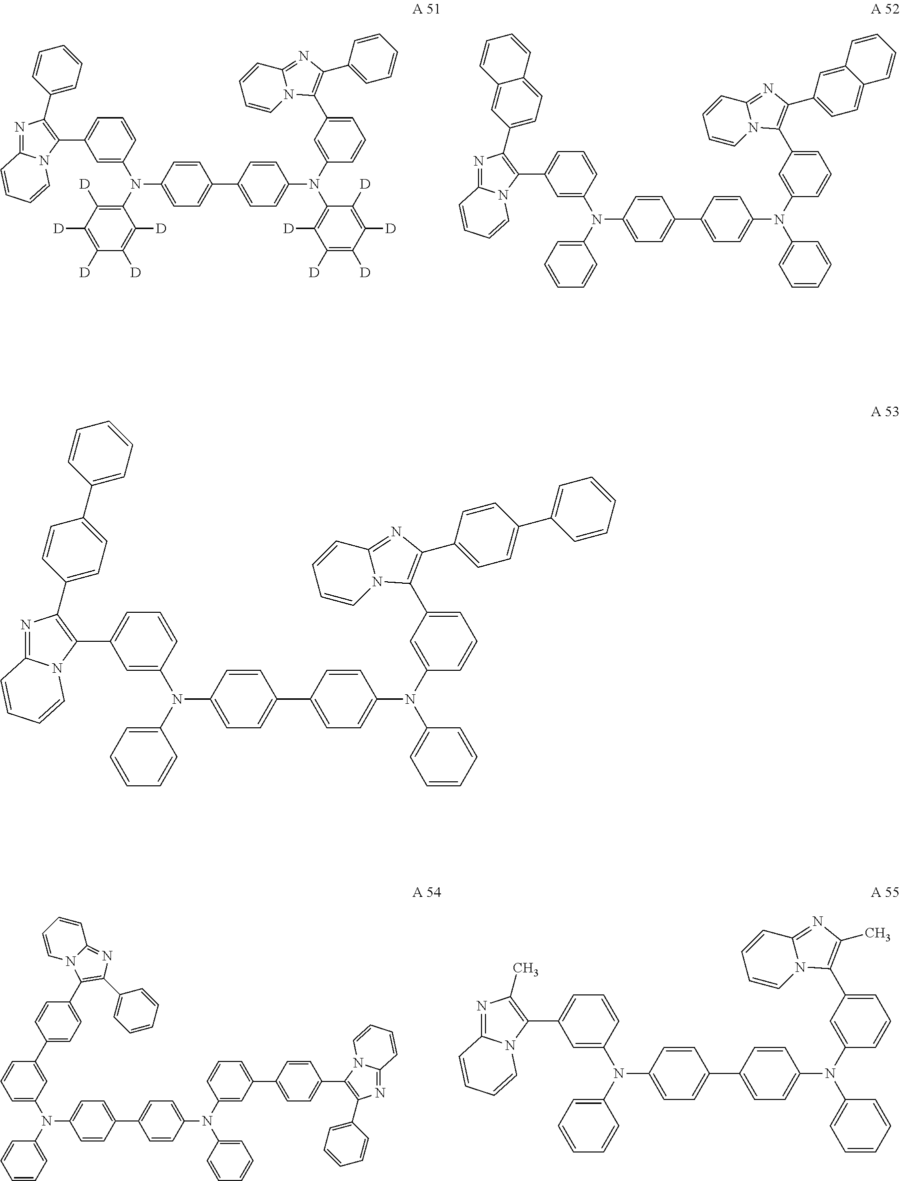

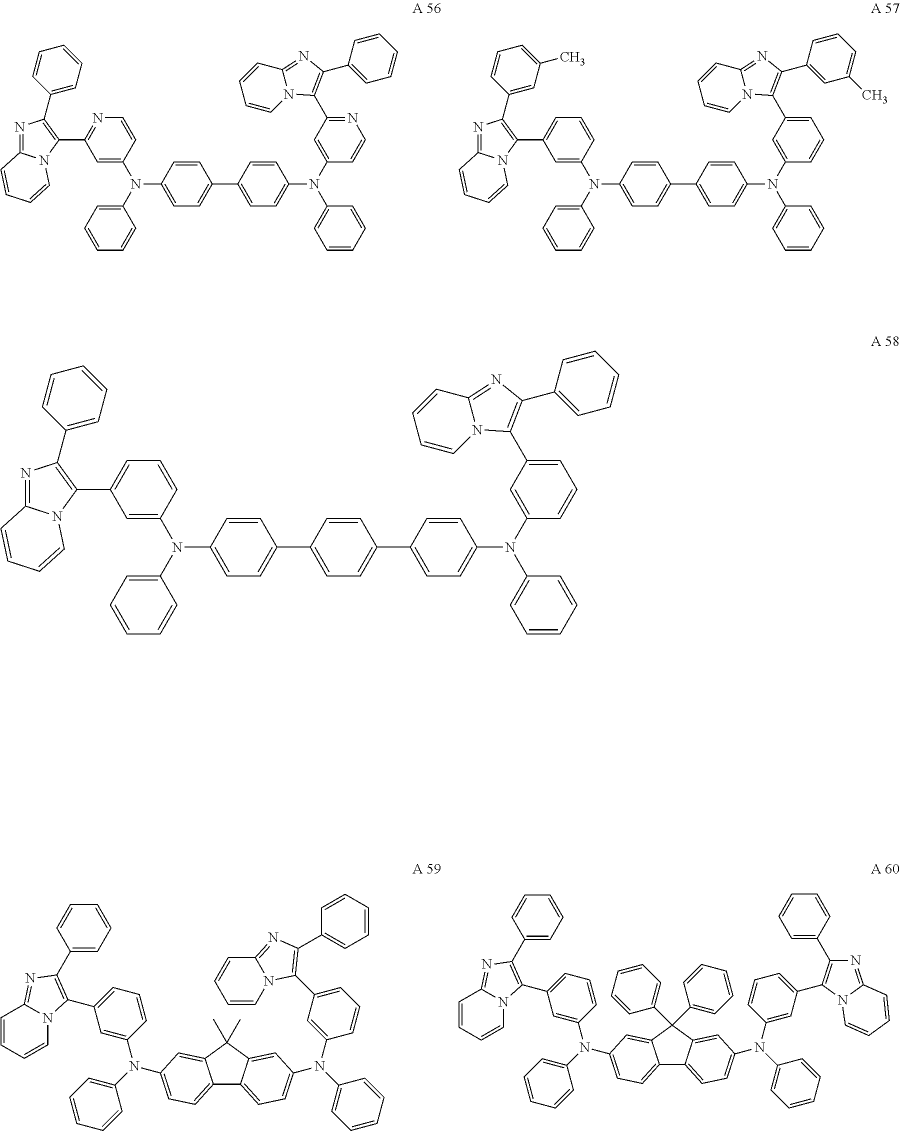

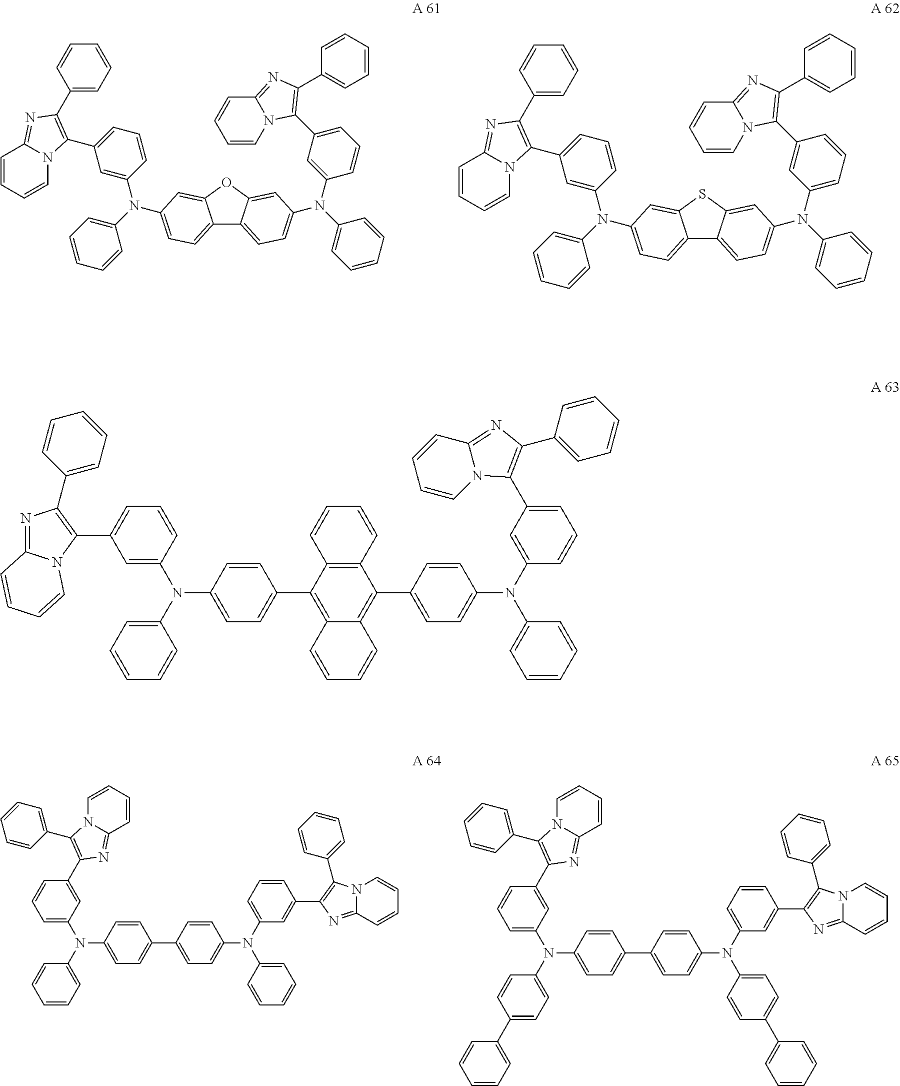

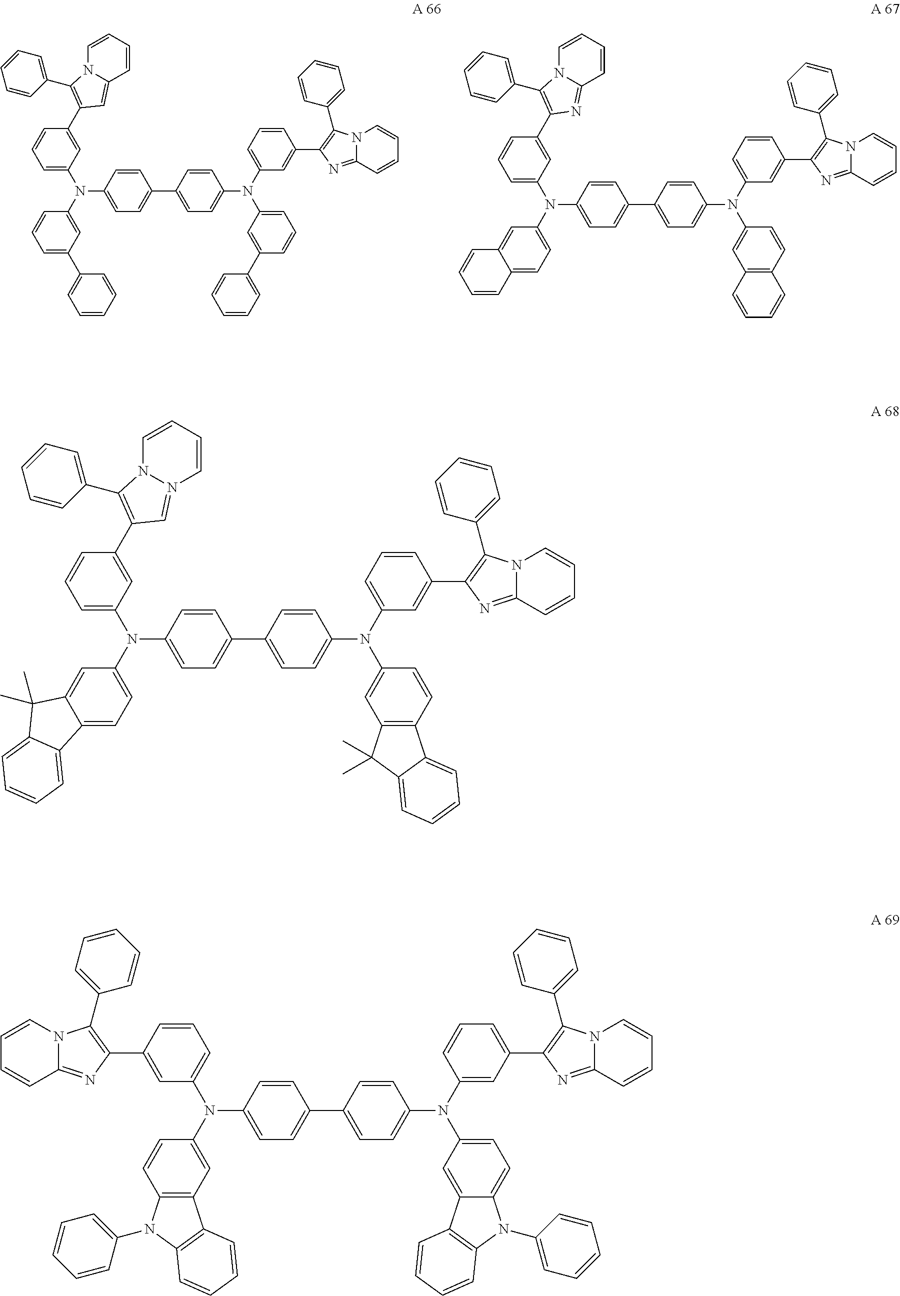

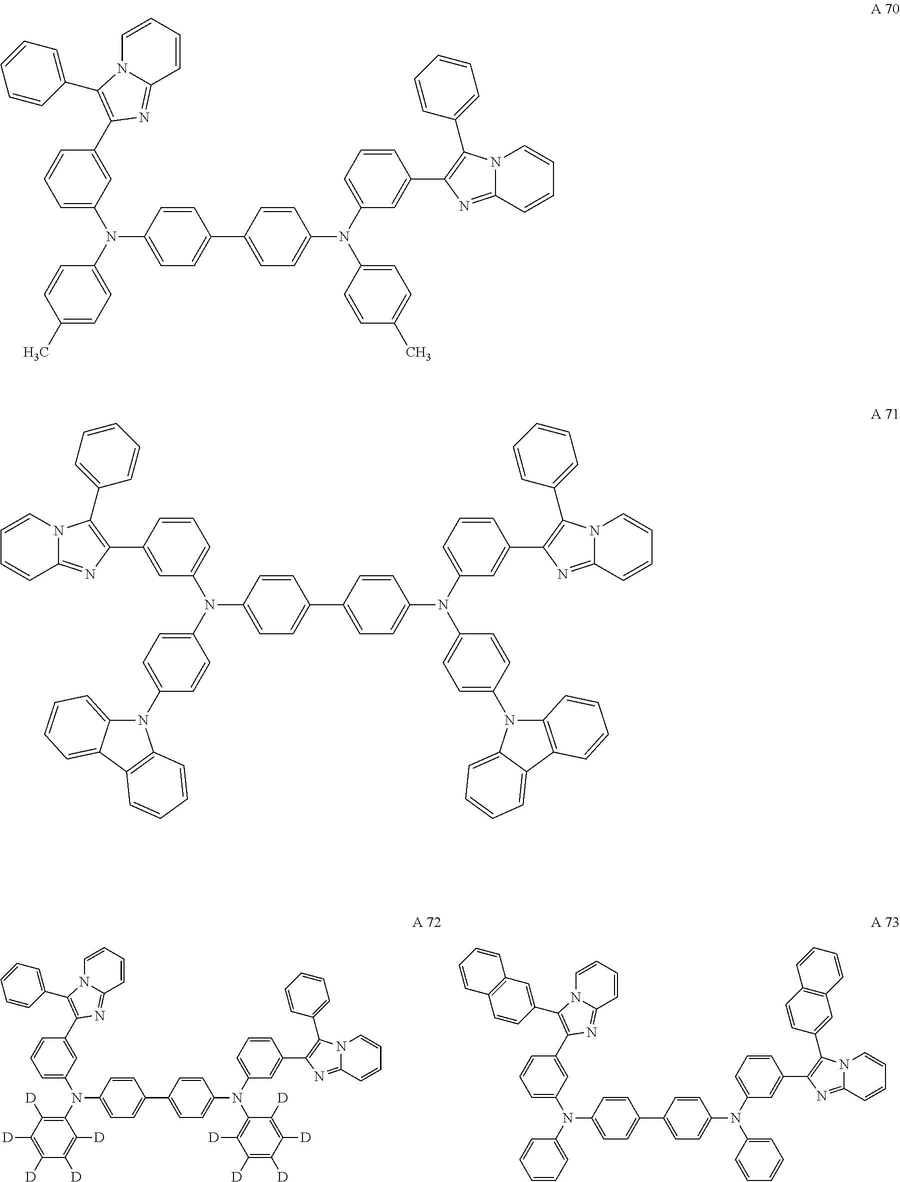

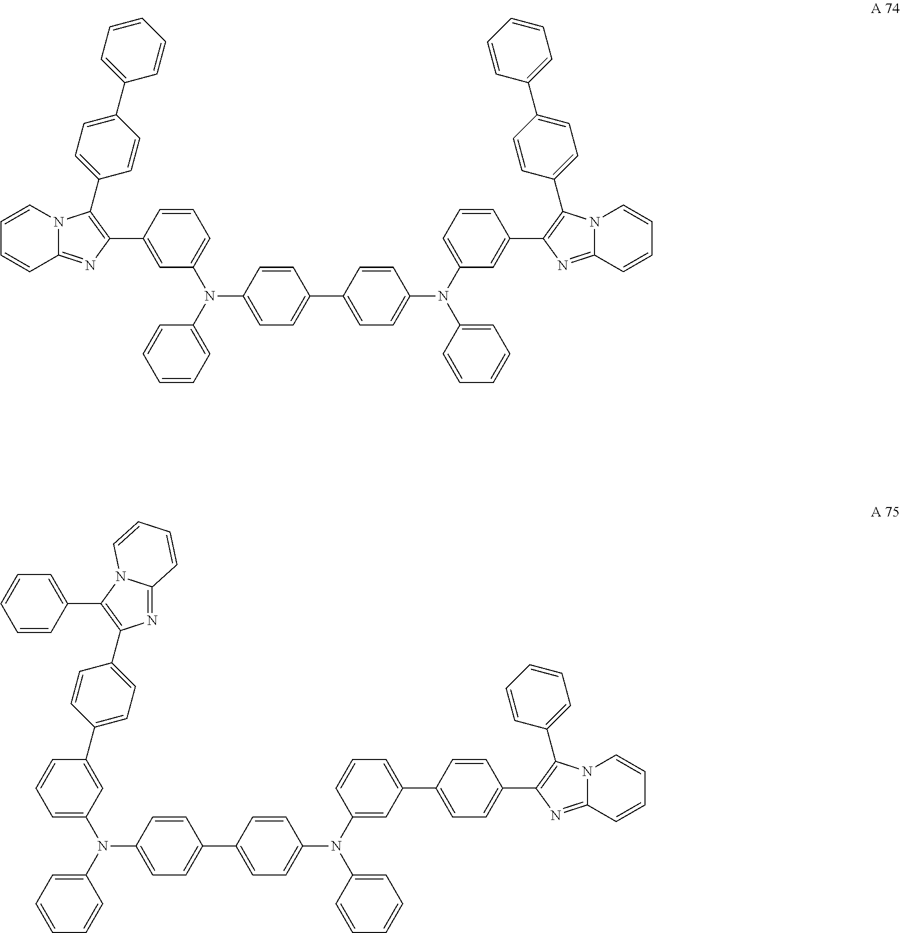 [SMILES: c1ccc(-c2ccc(-c3c(-c4cccc(N(c5ccccc5)c5ccc(-c6ccc(N(c7ccccc7)c7cccc(-c8nc9ccccn9c8-c8ccc(-c9ccccc9)cc8)c7)cc6)cc5)c4)nc4ccccn34)cc2)cc1 ; c1ccc(-c2c(-c3ccc(-c4cccc(N(c5ccccc5)c5ccc(-c6ccc(N(c7ccccc7)c7cccc(-c8ccc(-c9nc%10ccccn%10c9-c9ccccc9)cc8)c7)cc6)cc5)c4)cc3)nc3ccccn23)cc1]

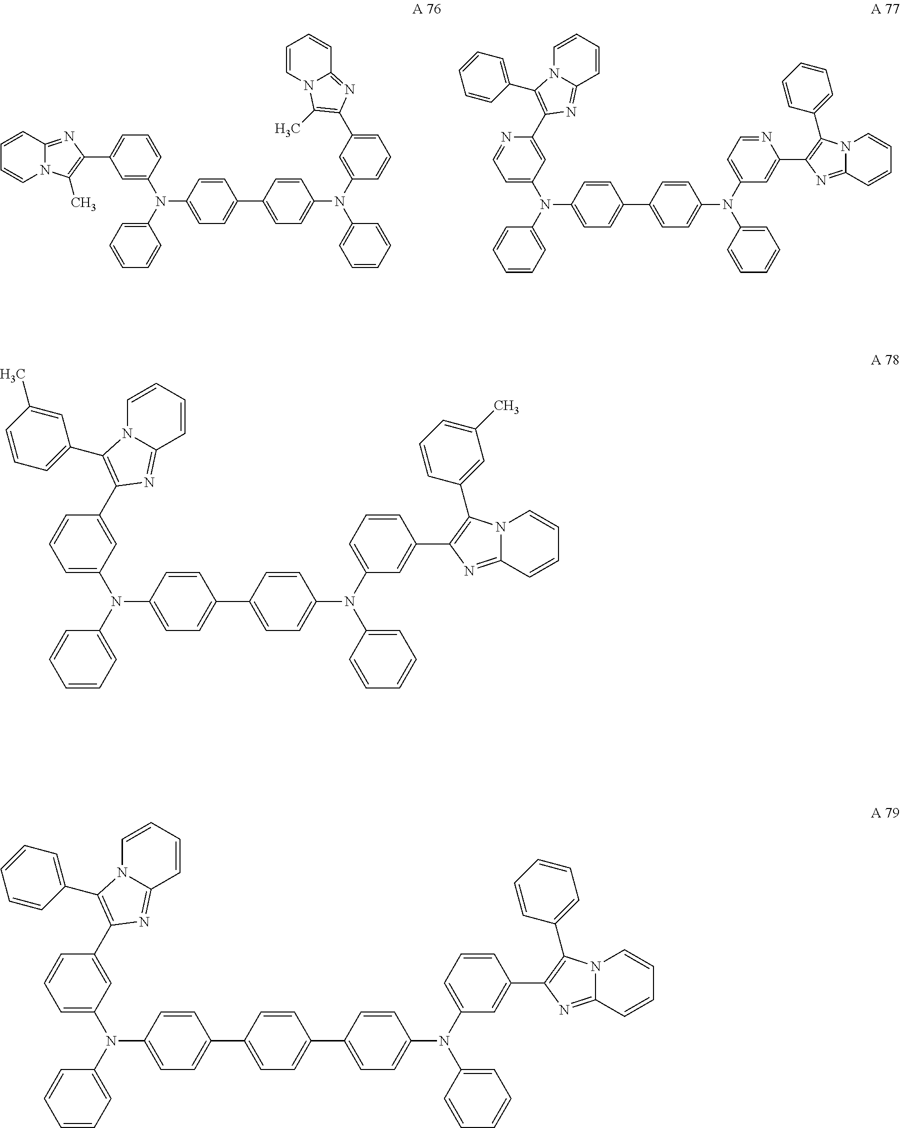 [SMILES: Cc1c(-c2cccc(N(c3ccccc3)c3ccc(-c4ccc(N(c5ccccc5)c5cccc(-c6nc7ccccn7c6C)c5)cc4)cc3)c2)nc2ccccn12 ; c1ccc(-c2c(-c3cc(N(c4ccccc4)c4ccc(-c5ccc(N(c6ccccc6)c6ccnc(-c7nc8ccccn8c7-c7ccccc7)c6)cc5)cc4)ccn3)nc3ccccn23)cc1 ; Cc1cccc(-c2c(-c3cccc(N(c4ccccc4)c4ccc(-c5ccc(N(c6ccccc6)c6cccc(-c7nc8ccccn8c7-c7cccc(C)c7)c6)cc5)cc4)c3)nc3ccccn23)c1 ; c1ccc(-c2c(-c3cccc(N(c4ccccc4)c4ccc(-c5ccc(-c6ccc(N(c7ccccc7)c7cccc(-c8nc9ccccn9c8-c8ccccc8)c7)cc6)cc5)cc4)c3)nc3ccccn23)cc1]

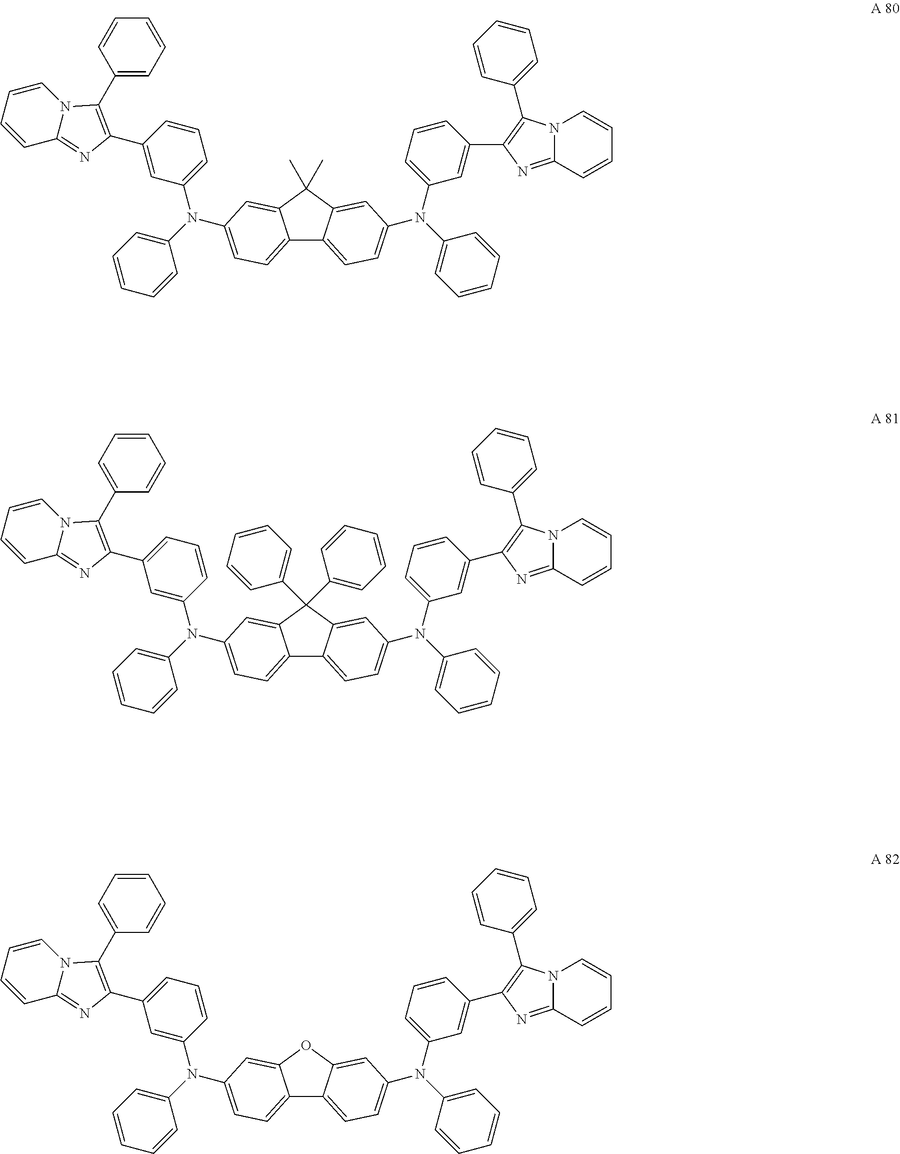 [SMILES: CC1(C)c2cc(N(c3ccccc3)c3cccc(-c4nc5ccccn5c4-c4ccccc4)c3)ccc2-c2ccc(N(c3ccccc3)c3cccc(-c4nc5ccccn5c4-c4ccccc4)c3)cc21 ; c1ccc(-c2c(-c3cccc(N(c4ccccc4)c4ccc5c(c4)C(c4ccccc4)(c4ccccc4)c4cc(N(c6ccccc6)c6cccc(-c7nc8ccccn8c7-c7ccccc7)c6)ccc4-5)c3)nc3ccccn23)cc1 ; c1ccc(-c2c(-c3cccc(N(c4ccccc4)c4ccc5c(c4)oc4cc(N(c6ccccc6)c6cccc(-c7nc8ccccn8c7-c7ccccc7)c6)ccc45)c3)nc3ccccn23)cc1]

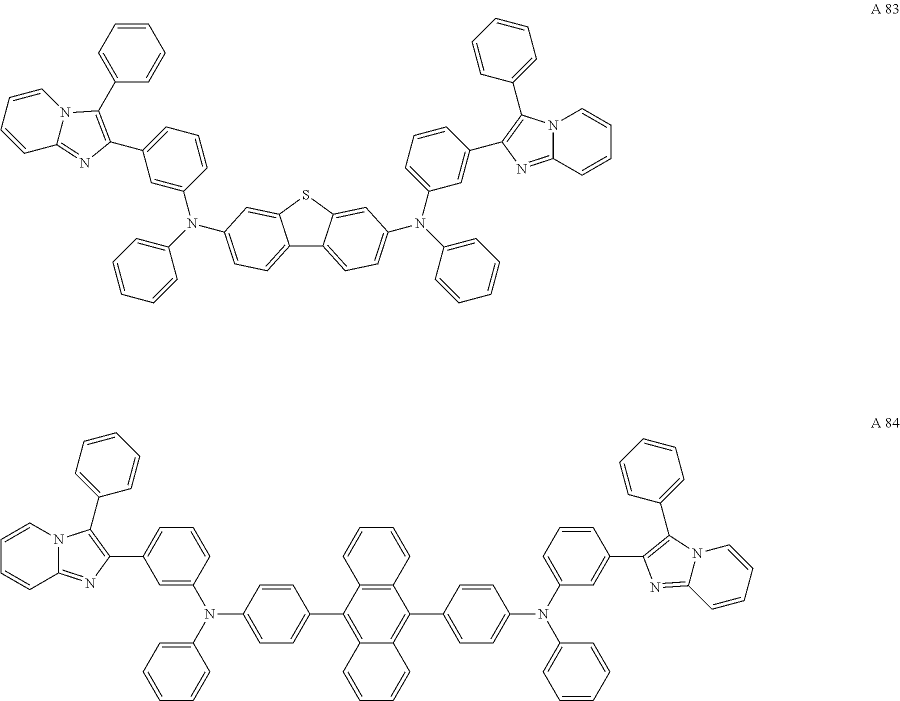 [SMILES: c1ccc(-c2c(-c3cccc(N(c4ccccc4)c4ccc5c(c4)sc4cc(N(c6ccccc6)c6cccc(-c7nc8ccccn8c7-c7ccccc7)c6)ccc45)c3)nc3ccccn23)cc1 ; c1ccc(-c2c(-c3cccc(N(c4ccccc4)c4ccc(-c5c6ccccc6c(-c6ccc(N(c7ccccc7)c7cccc(-c8nc9ccccn9c8-c8ccccc8)c7)cc6)c6ccccc56)cc4)c3)nc3ccccn23)cc1]

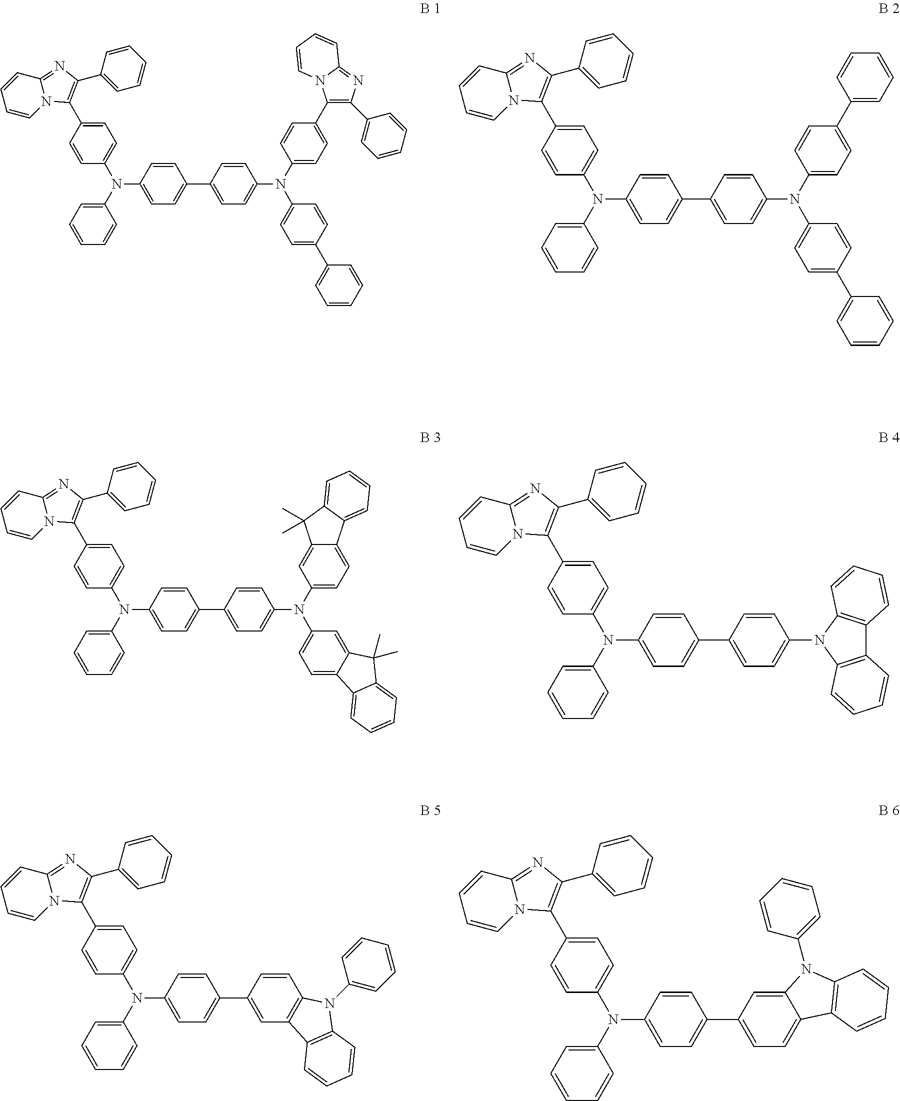

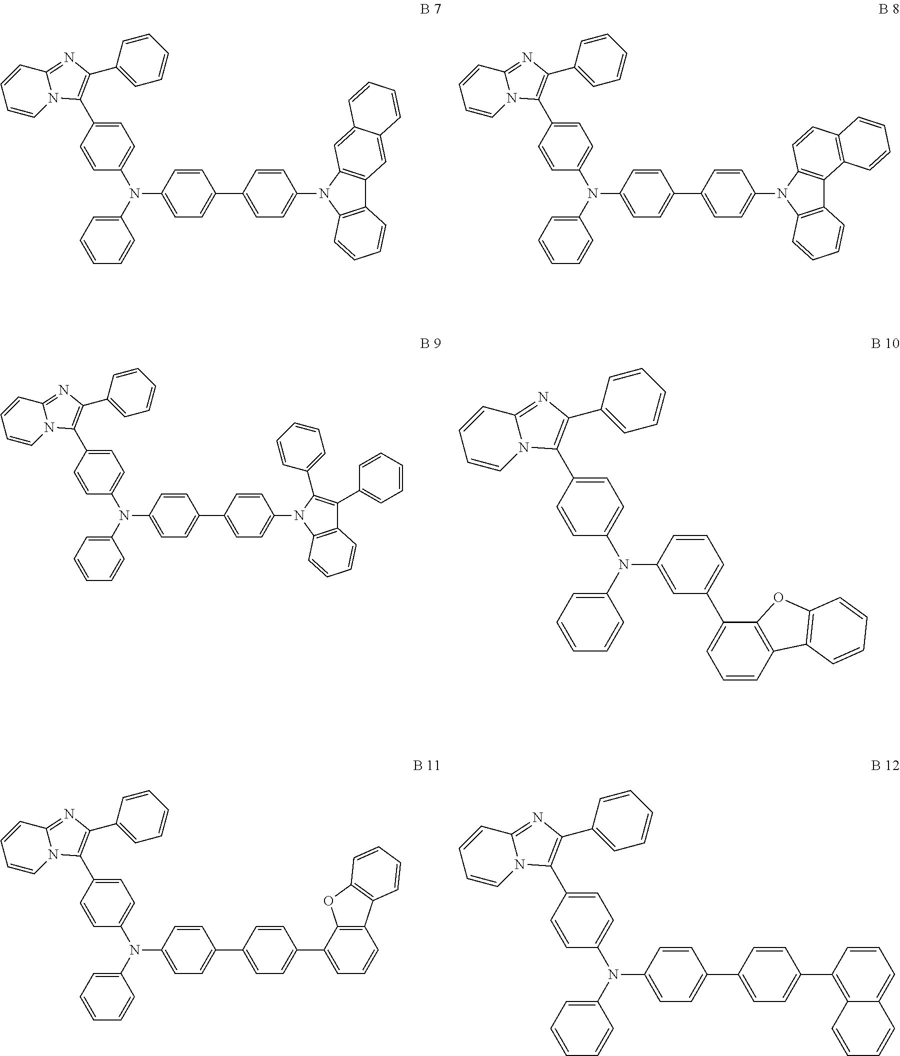

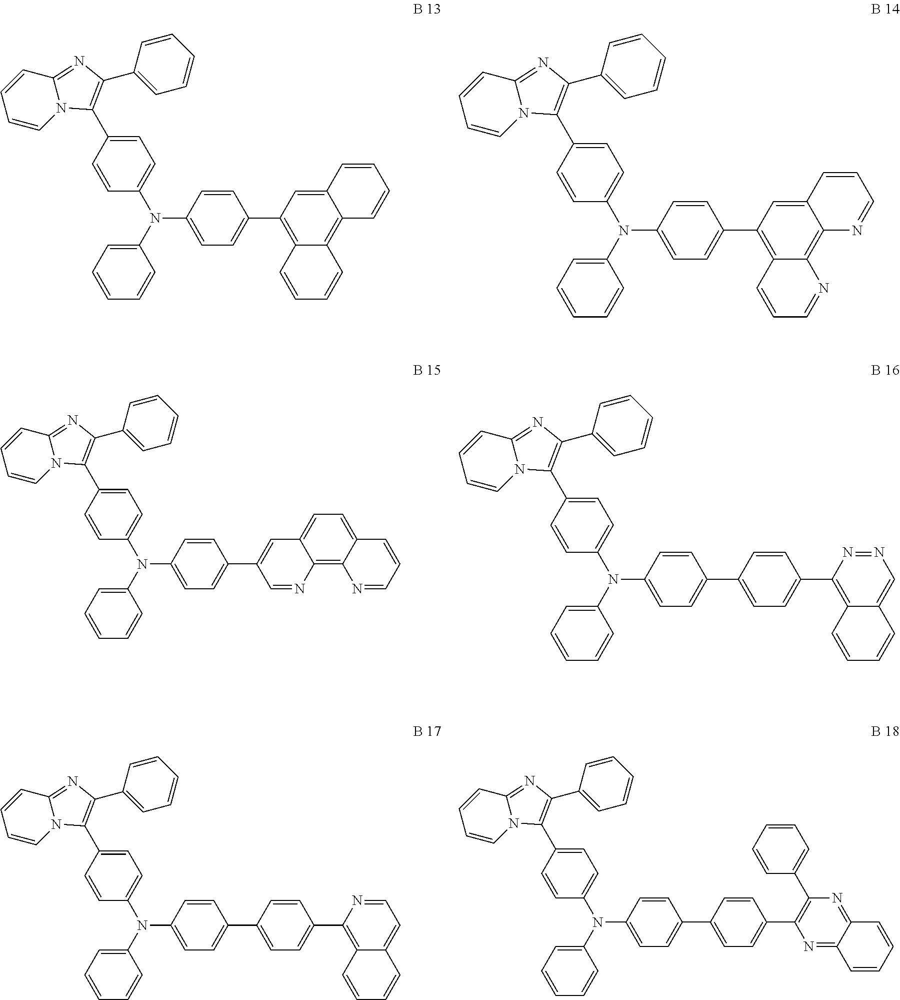

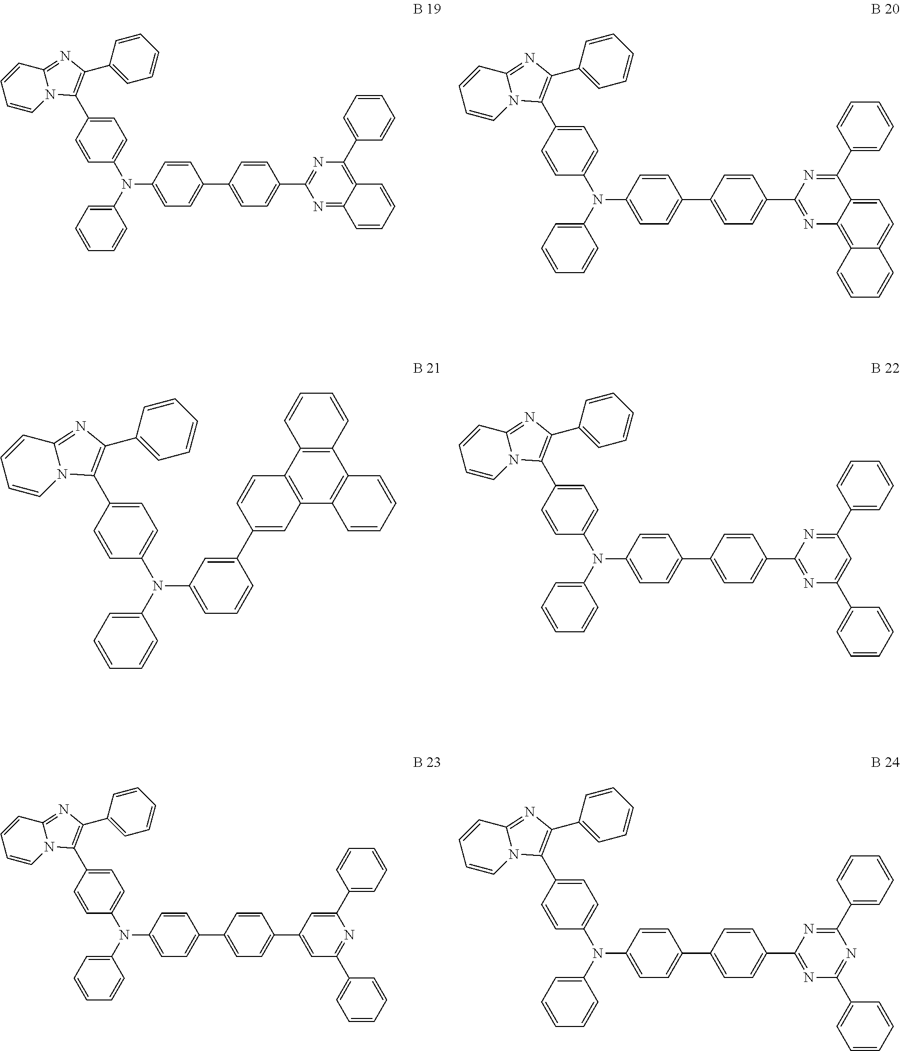

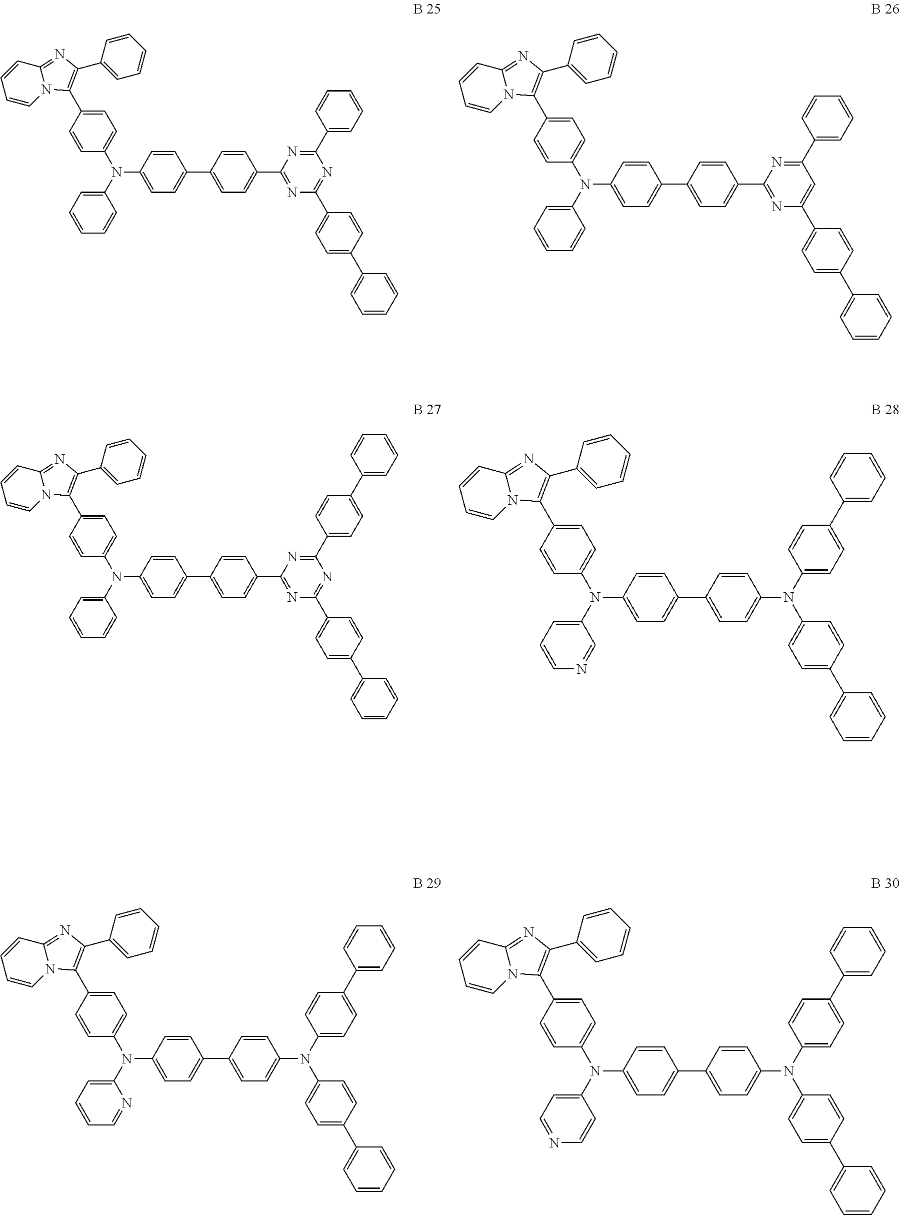

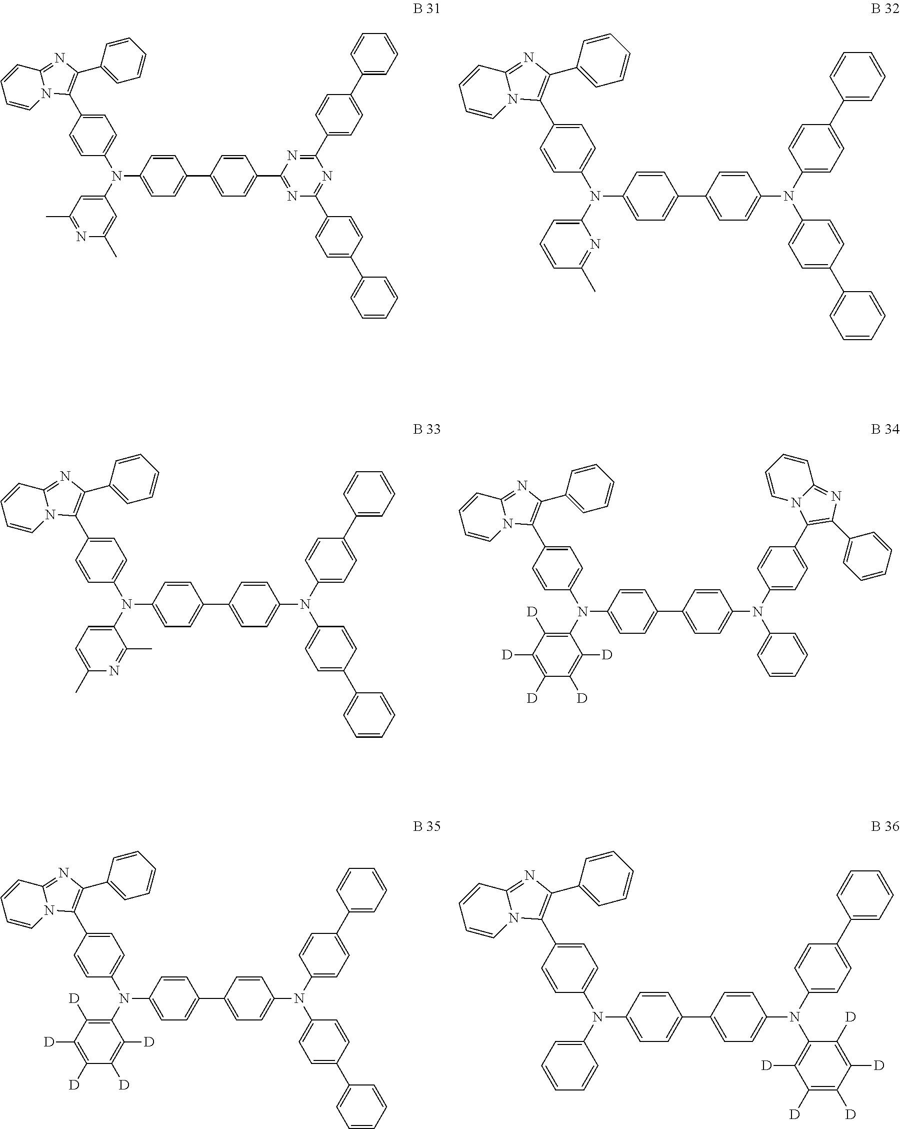

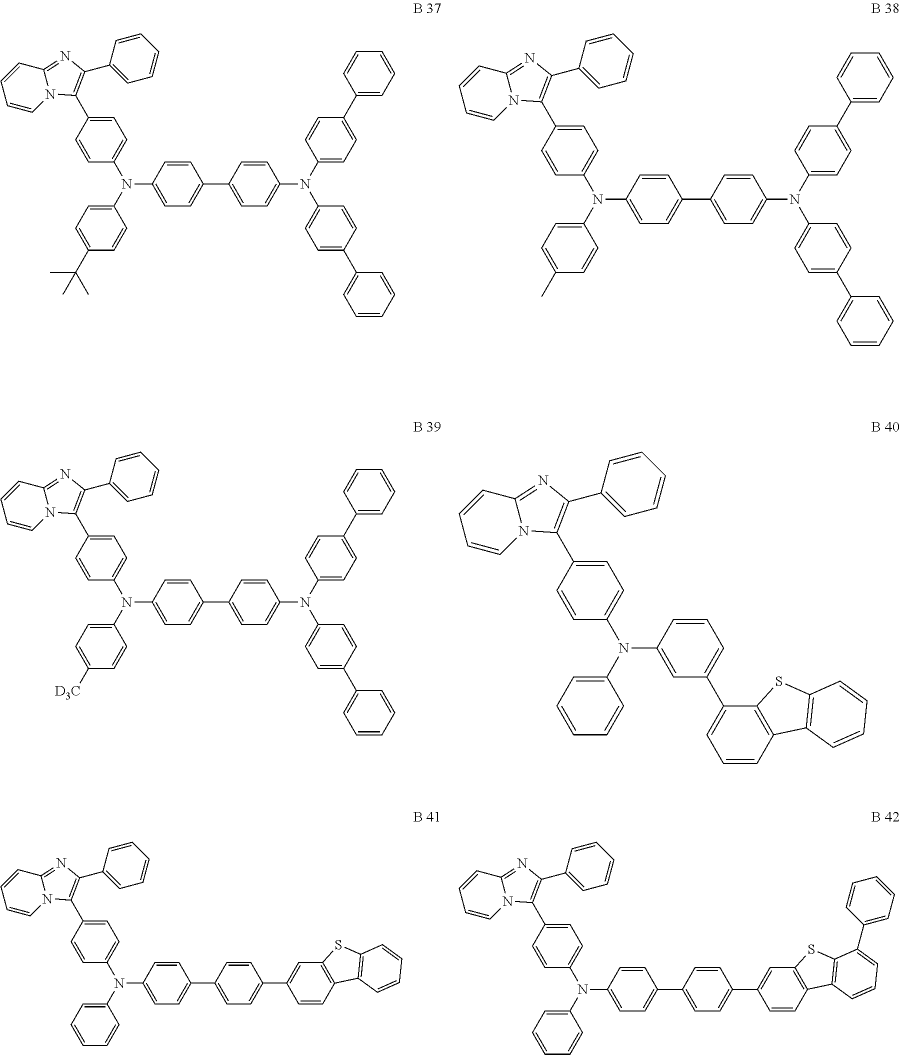

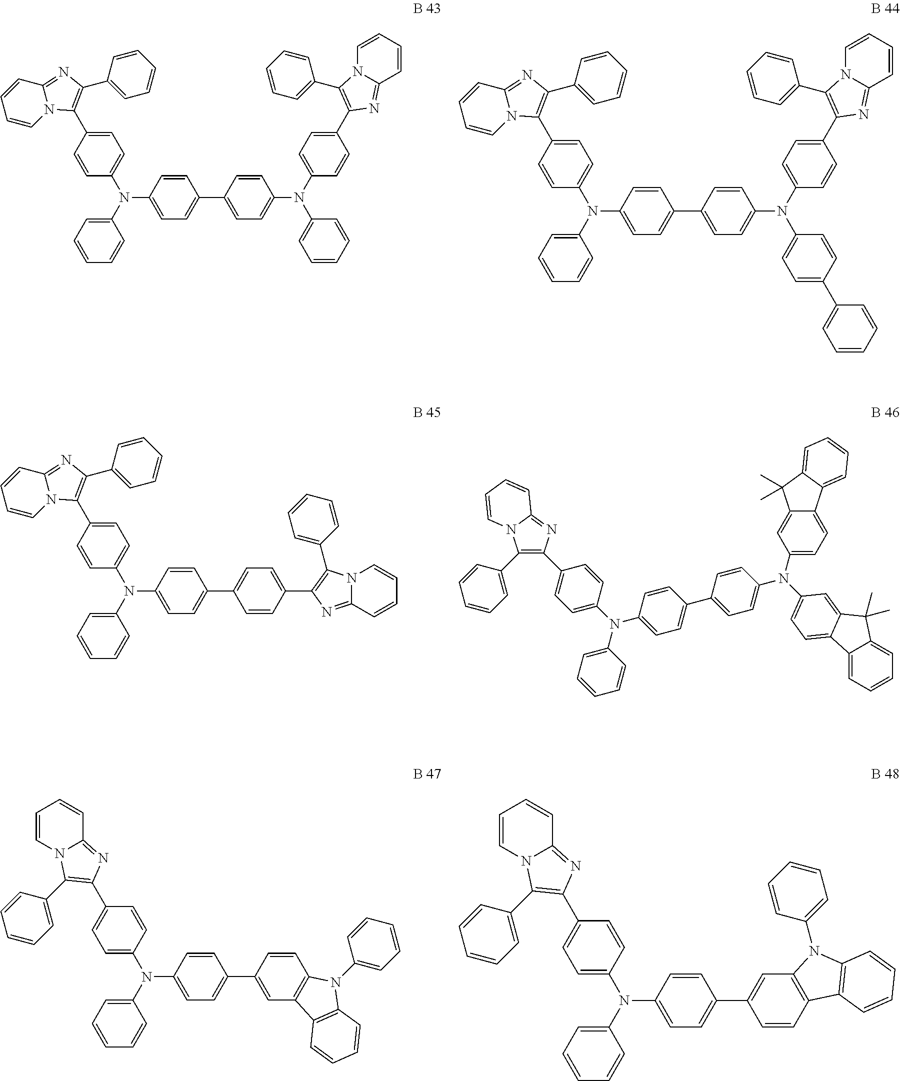

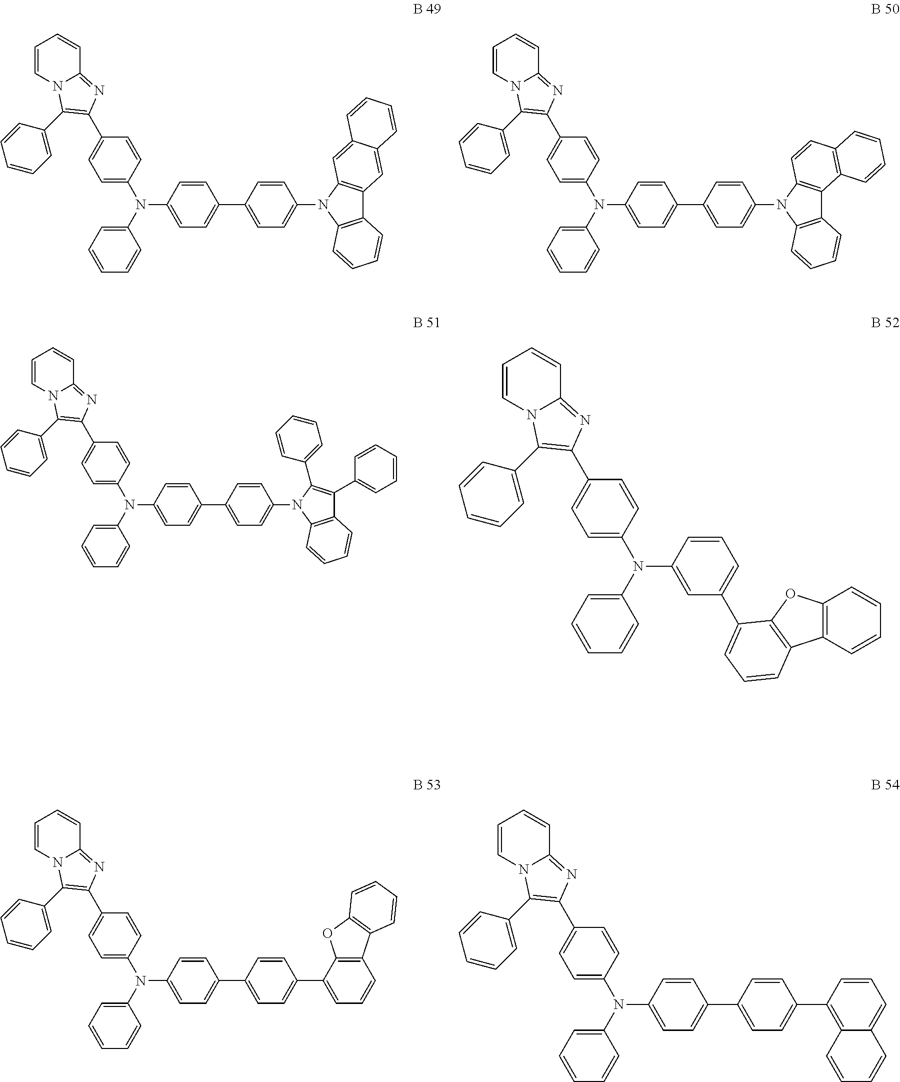

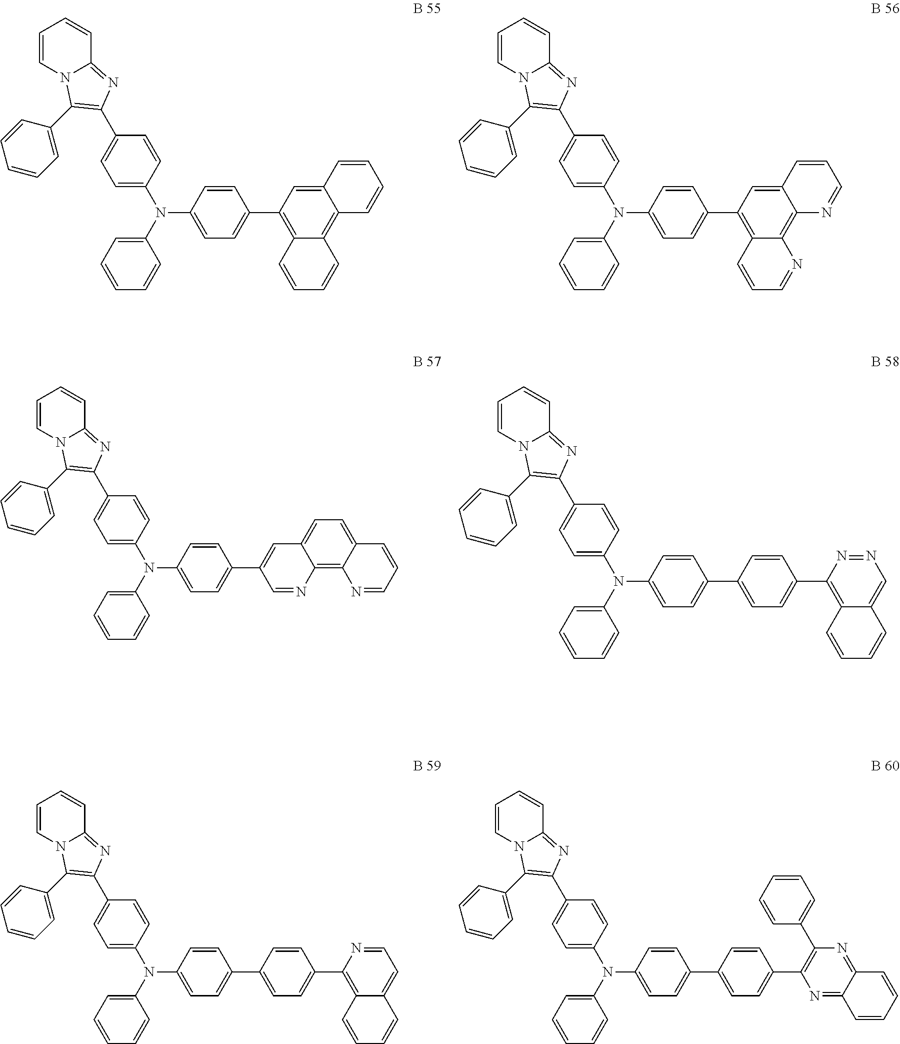

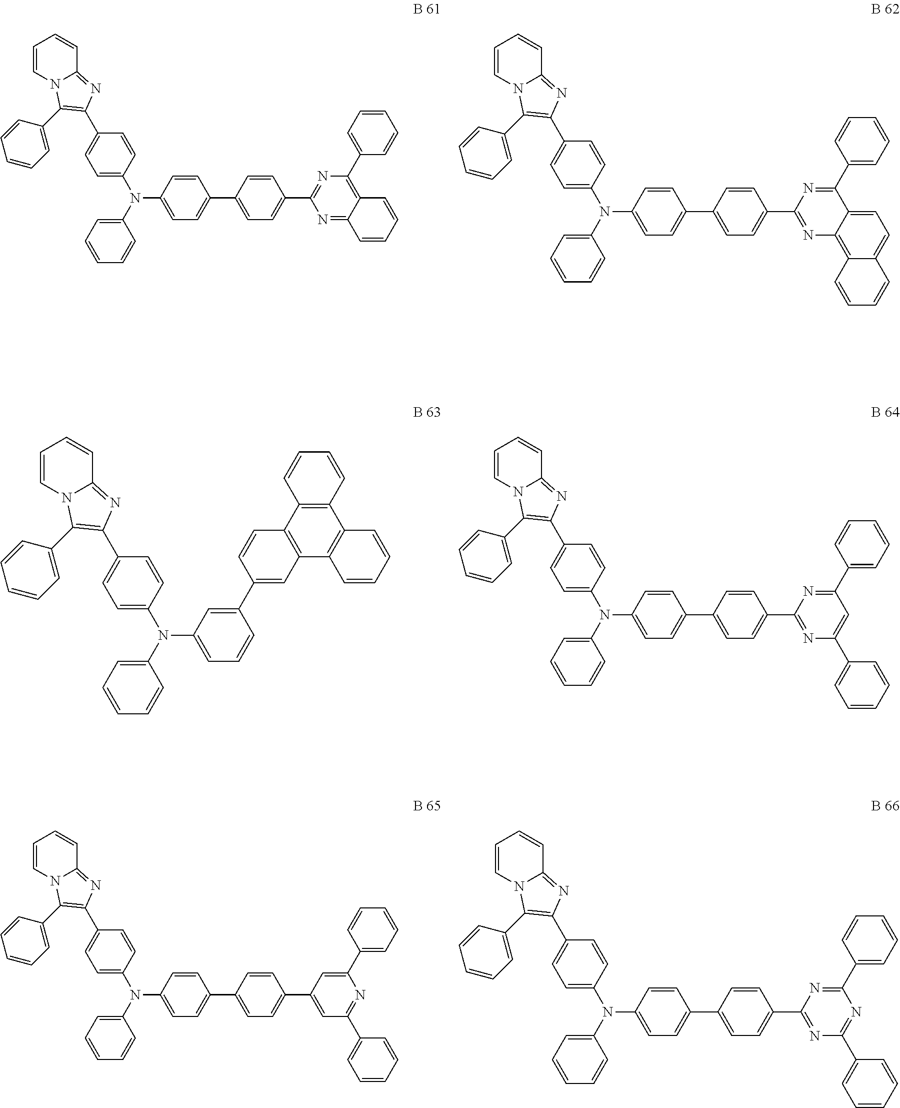

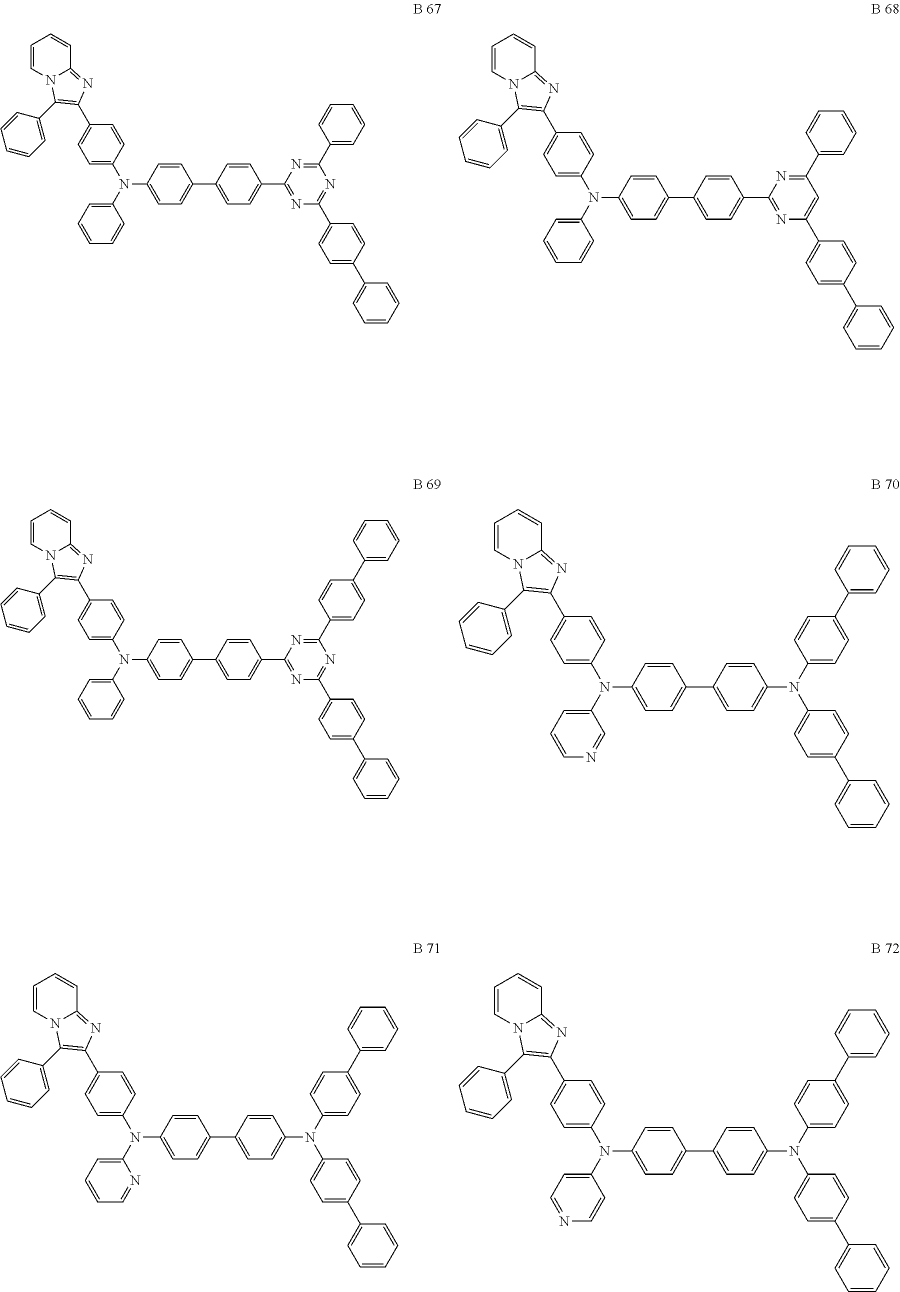

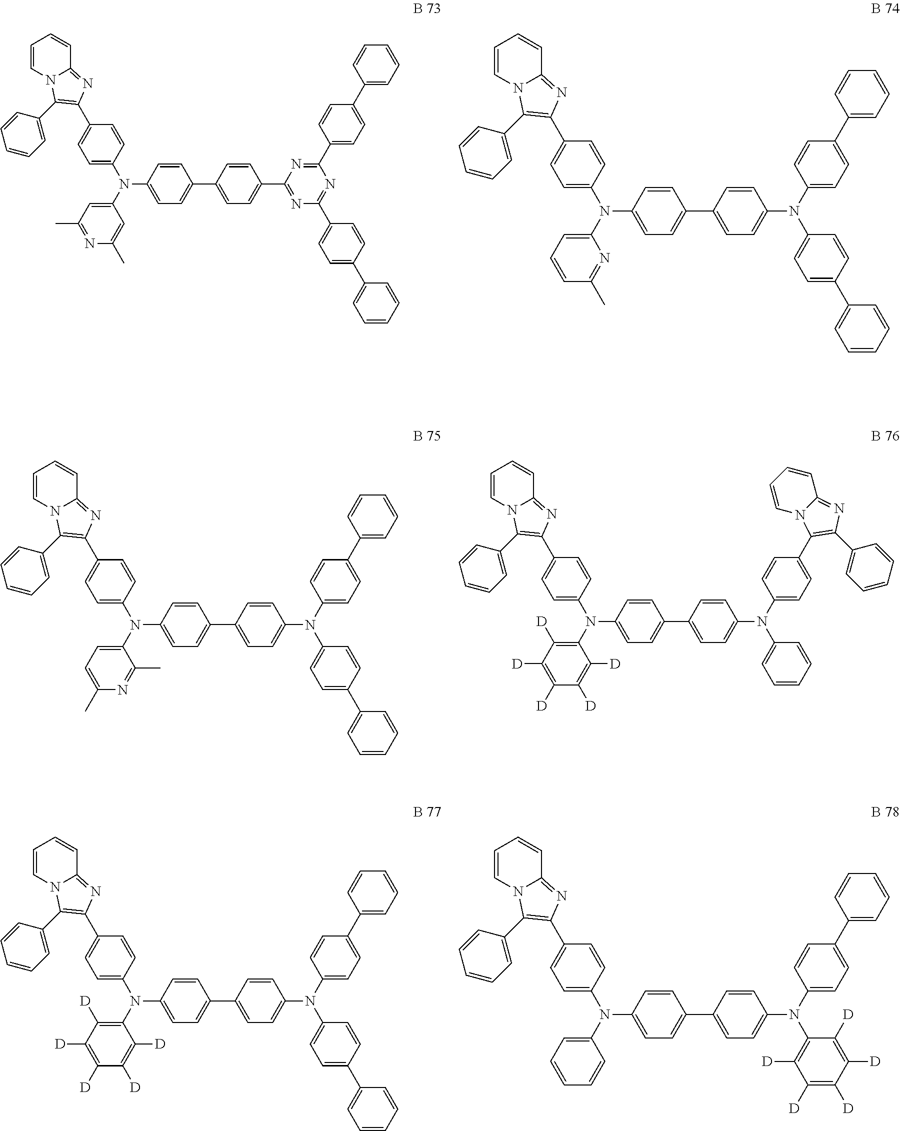

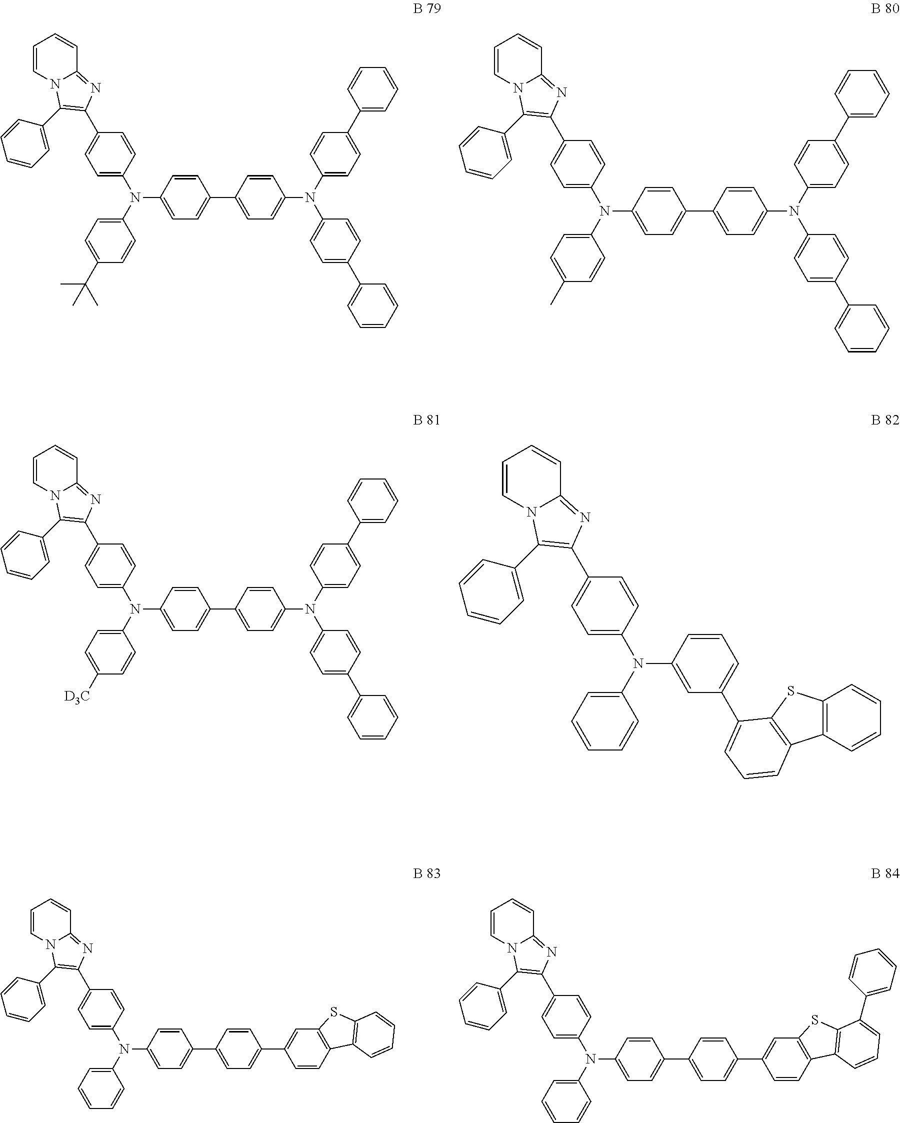

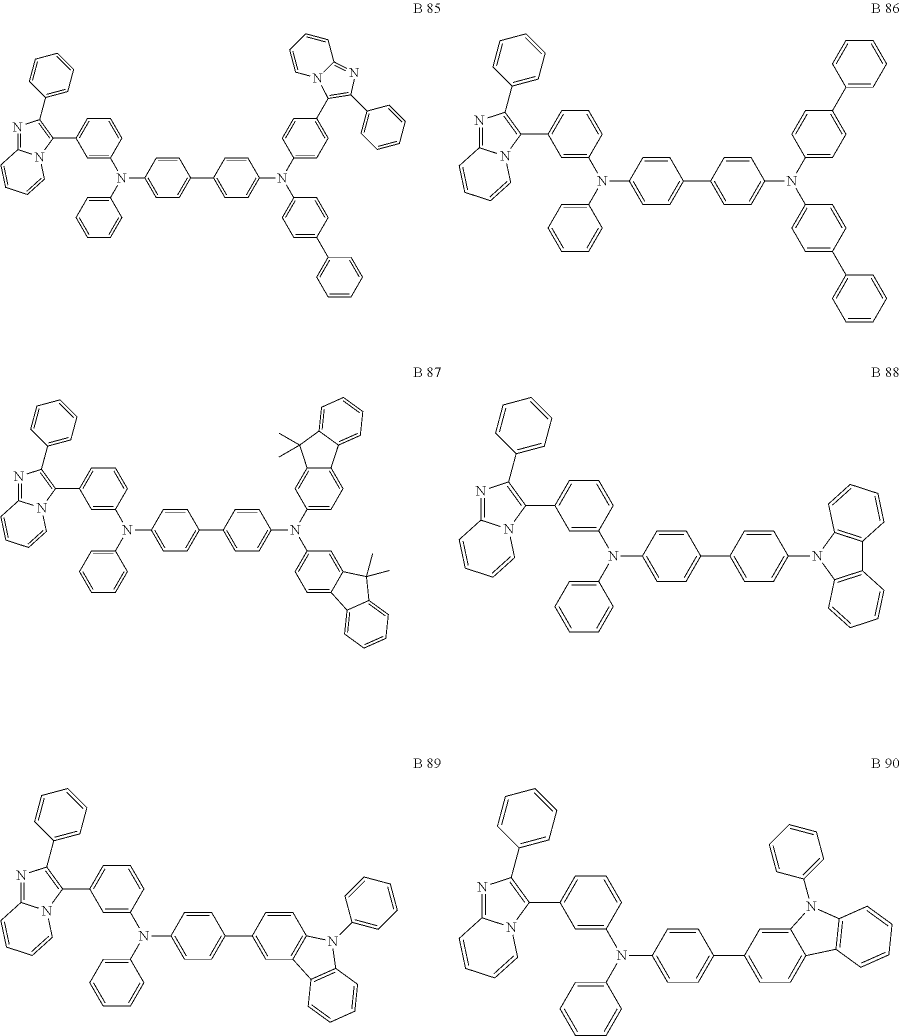

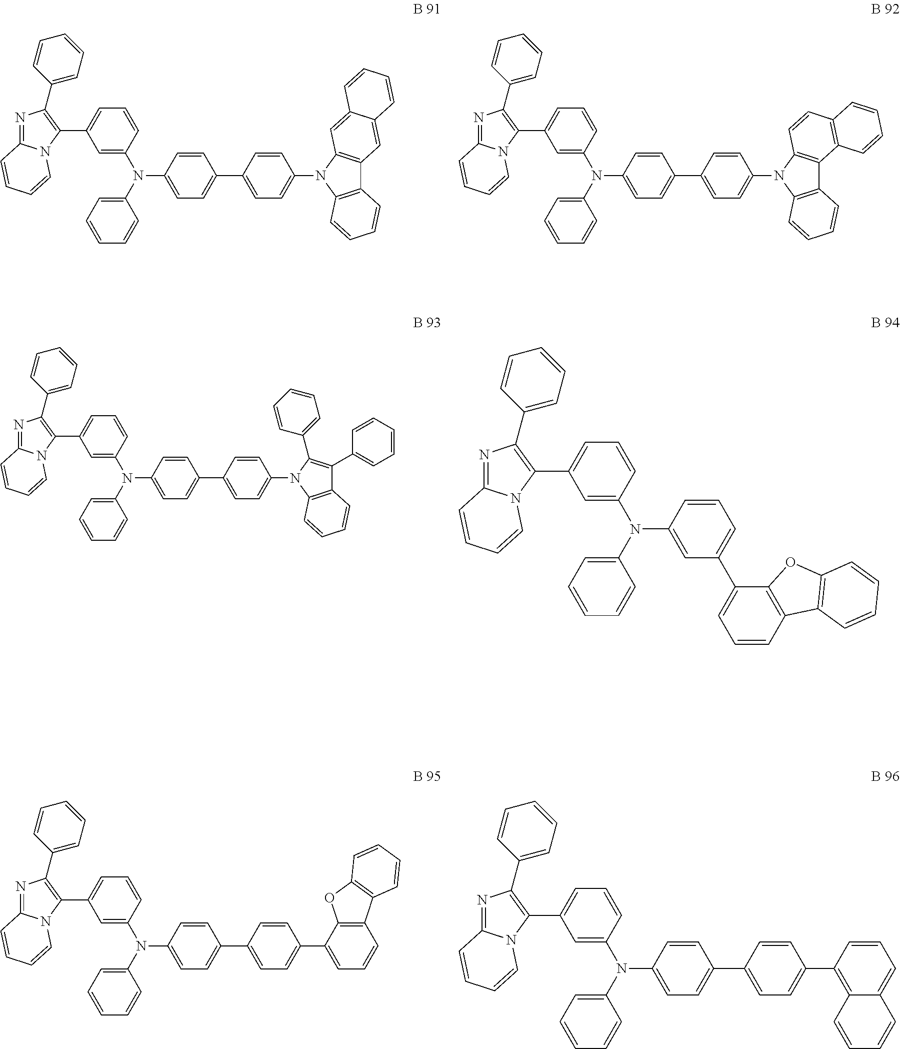

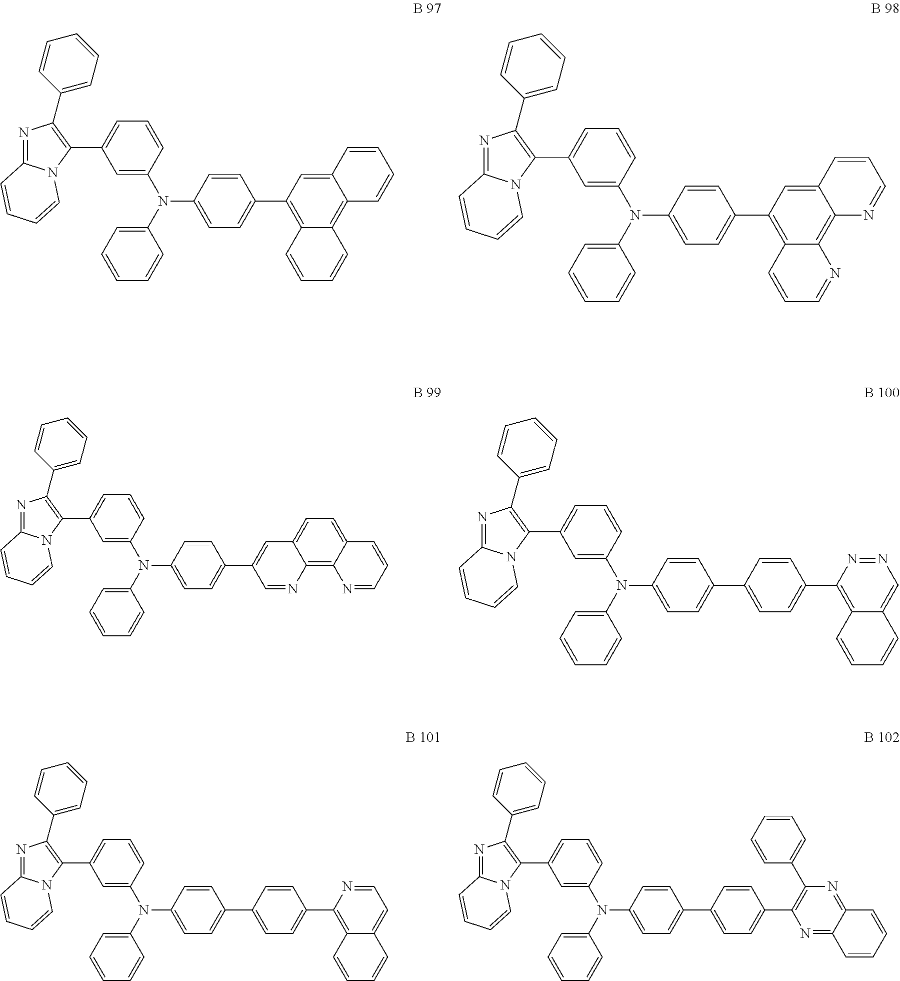

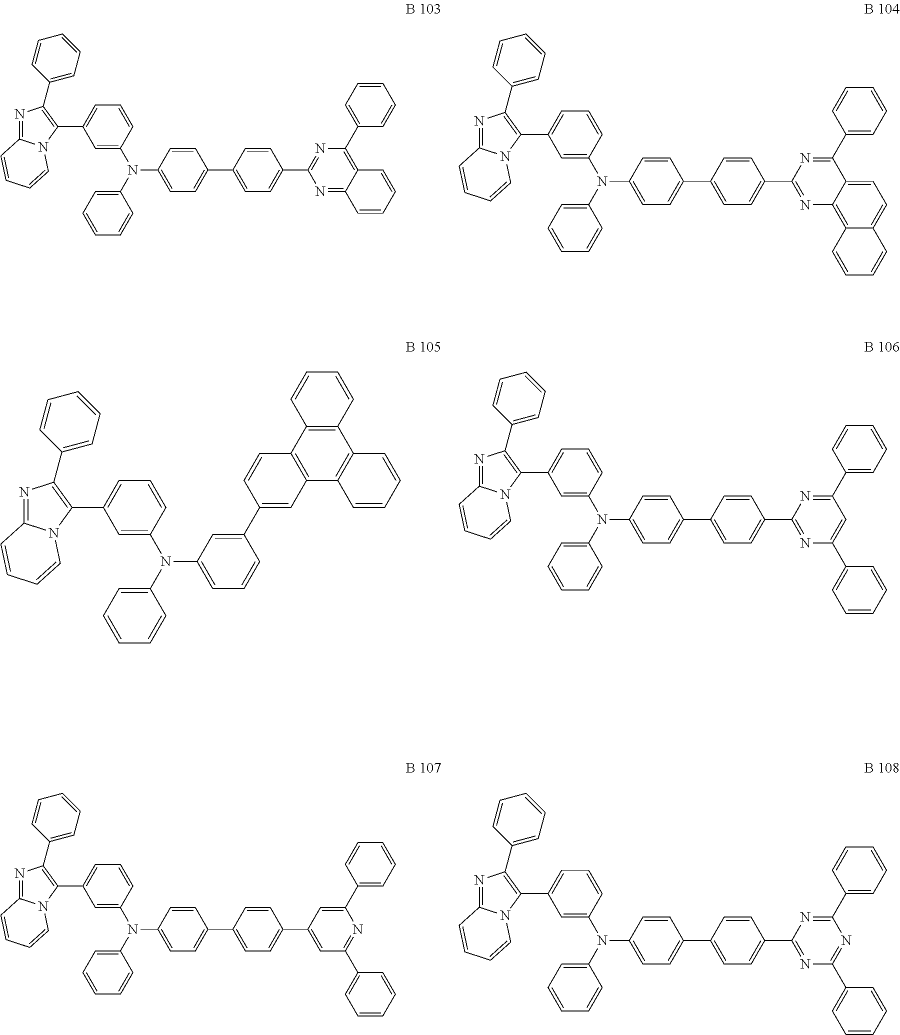

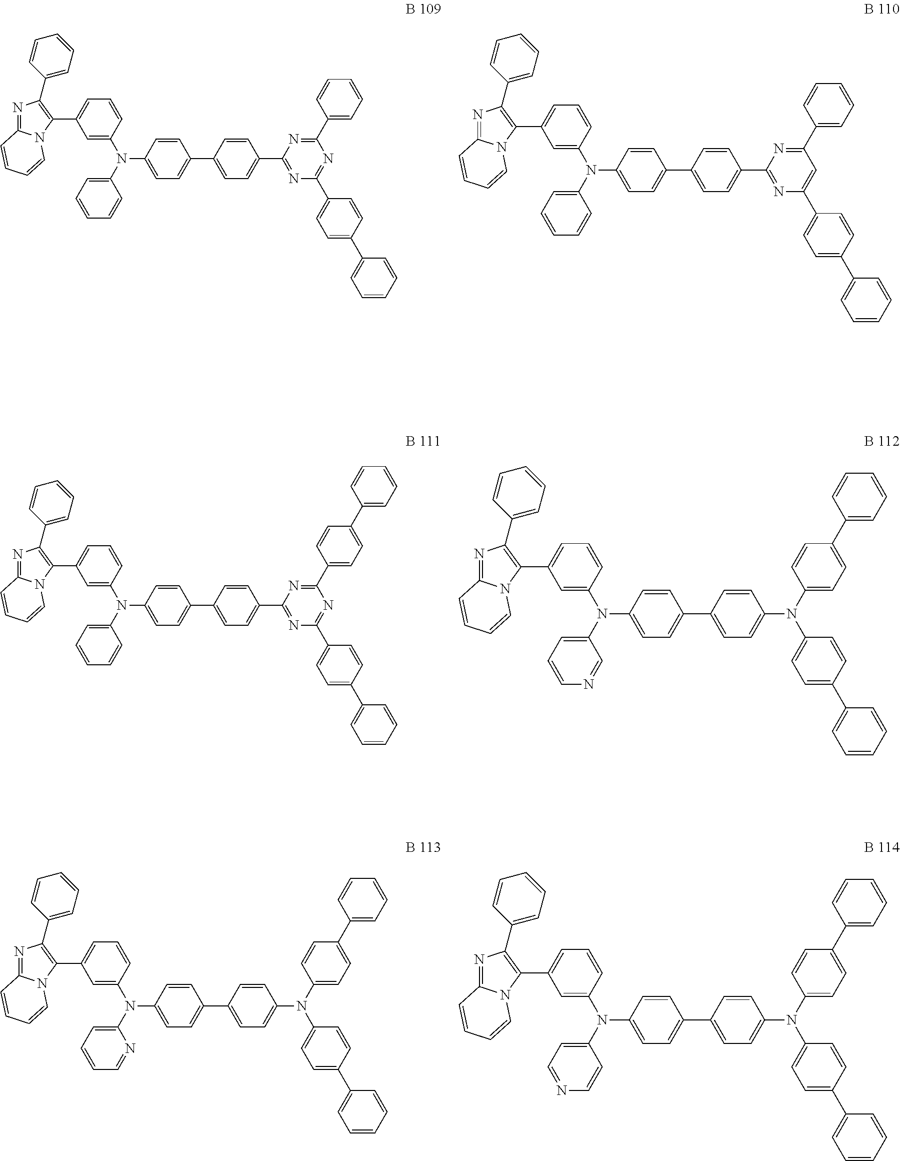

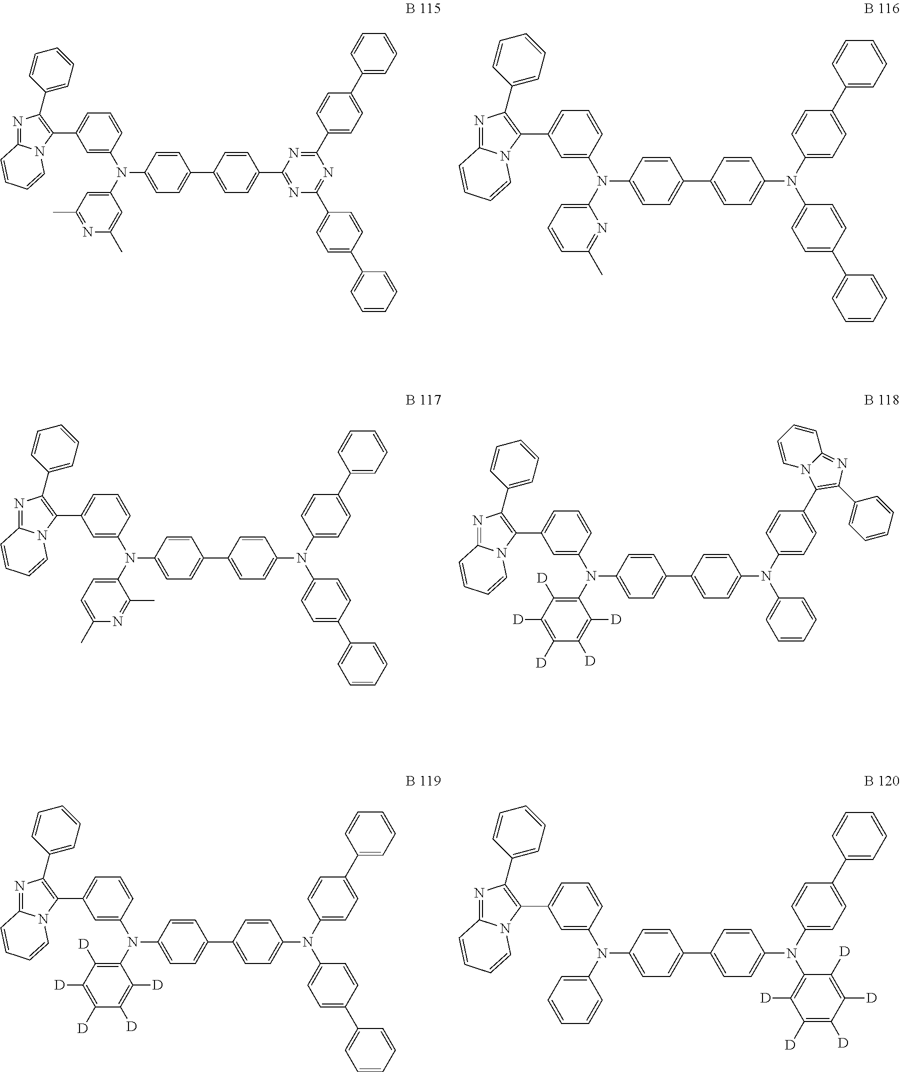

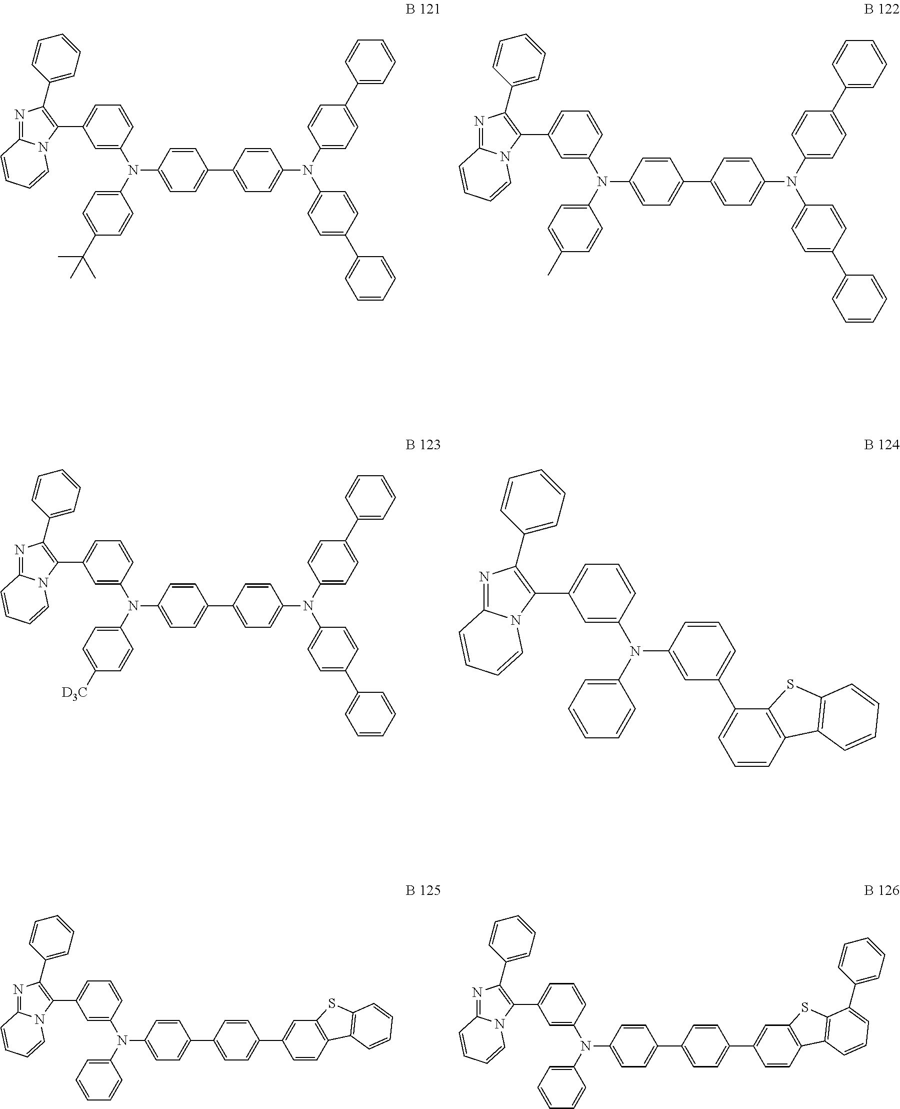

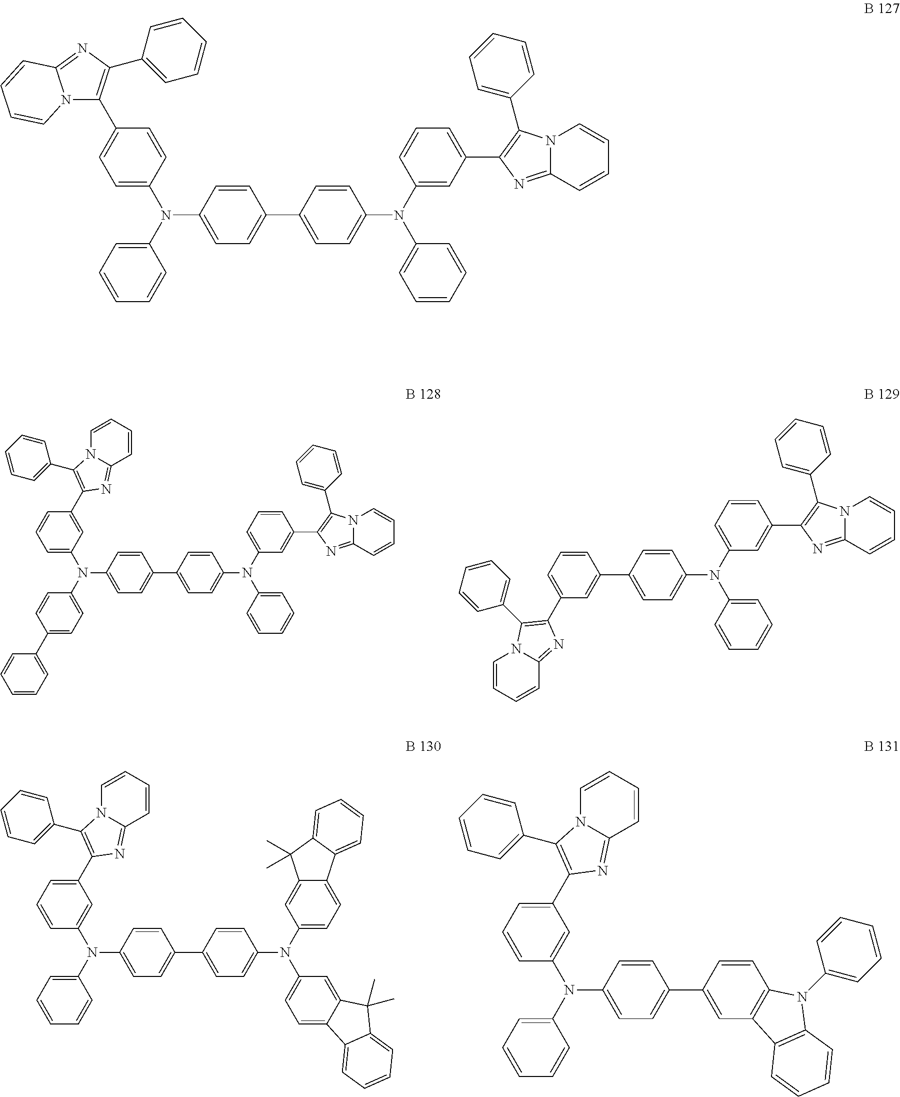

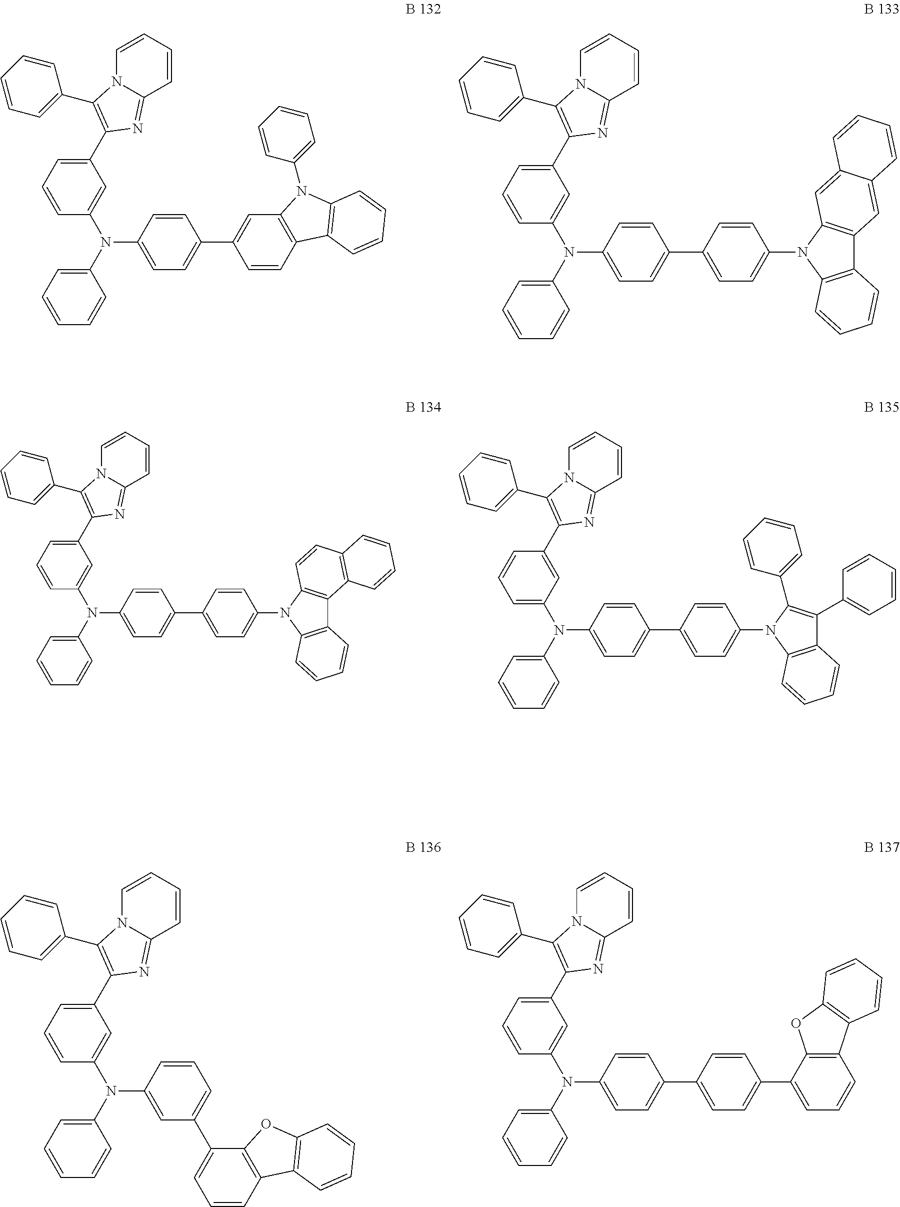

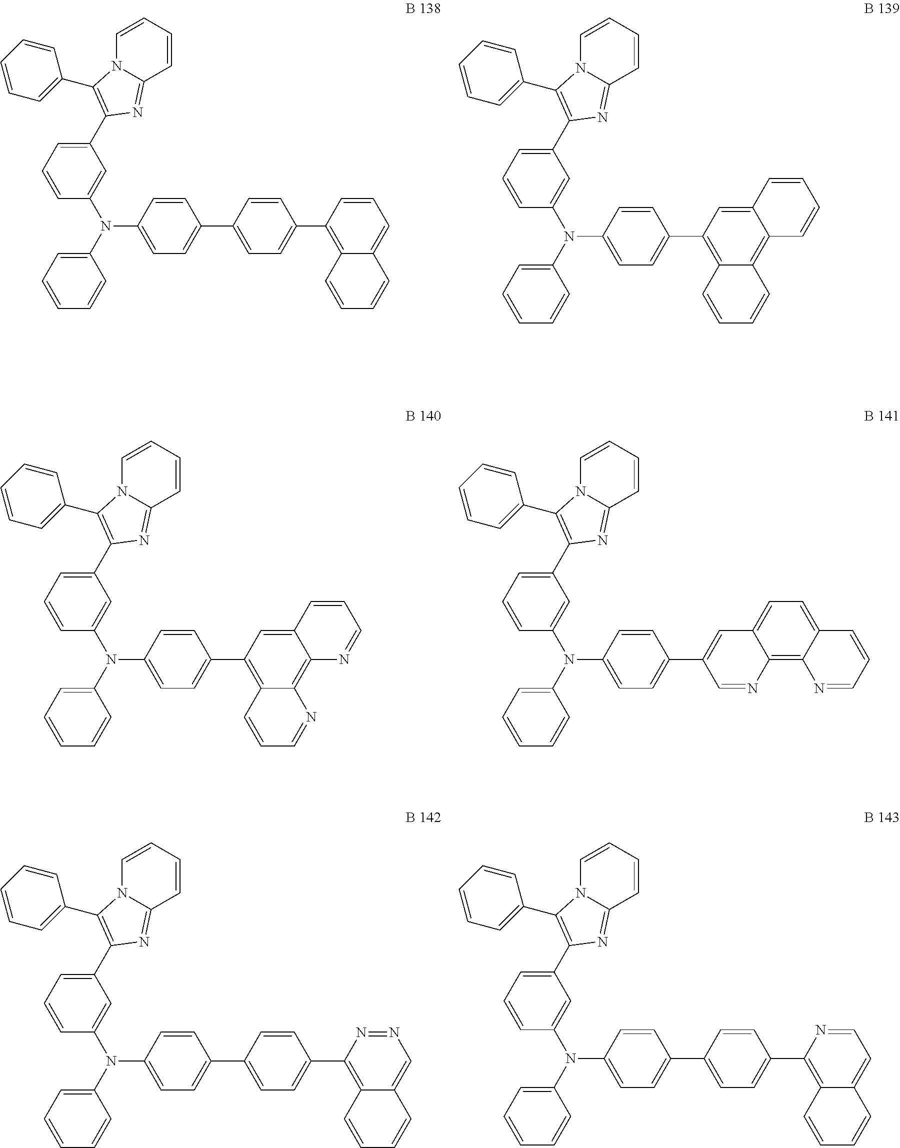

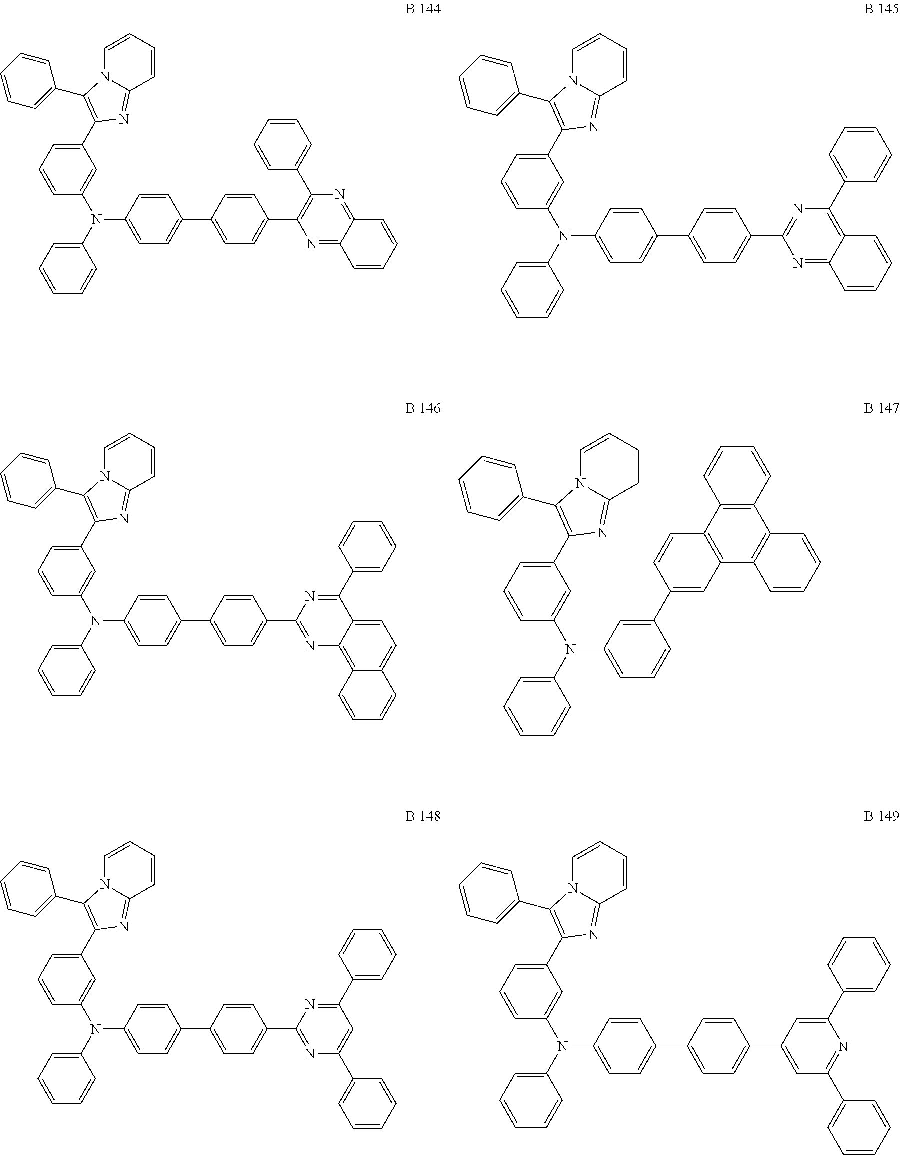

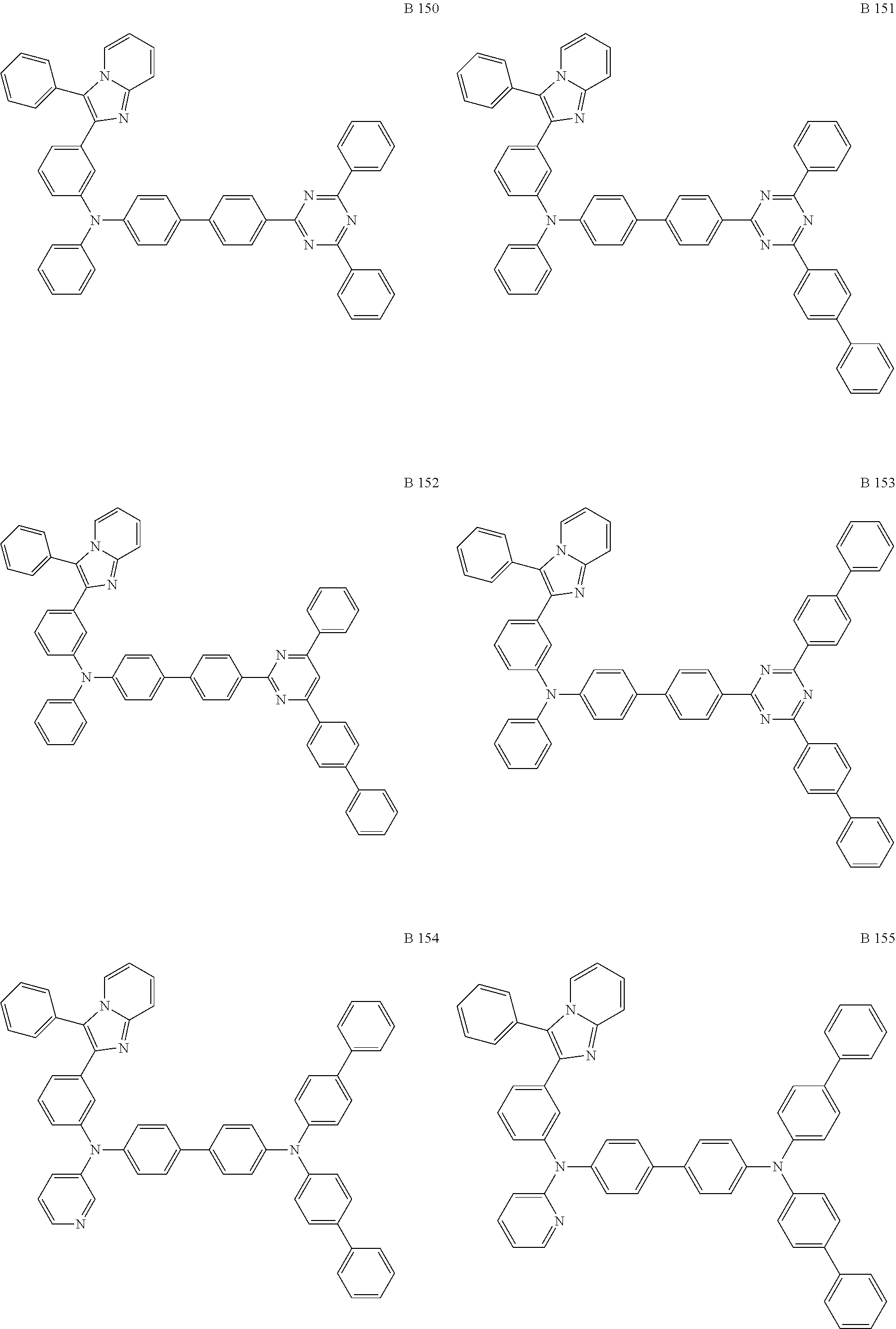

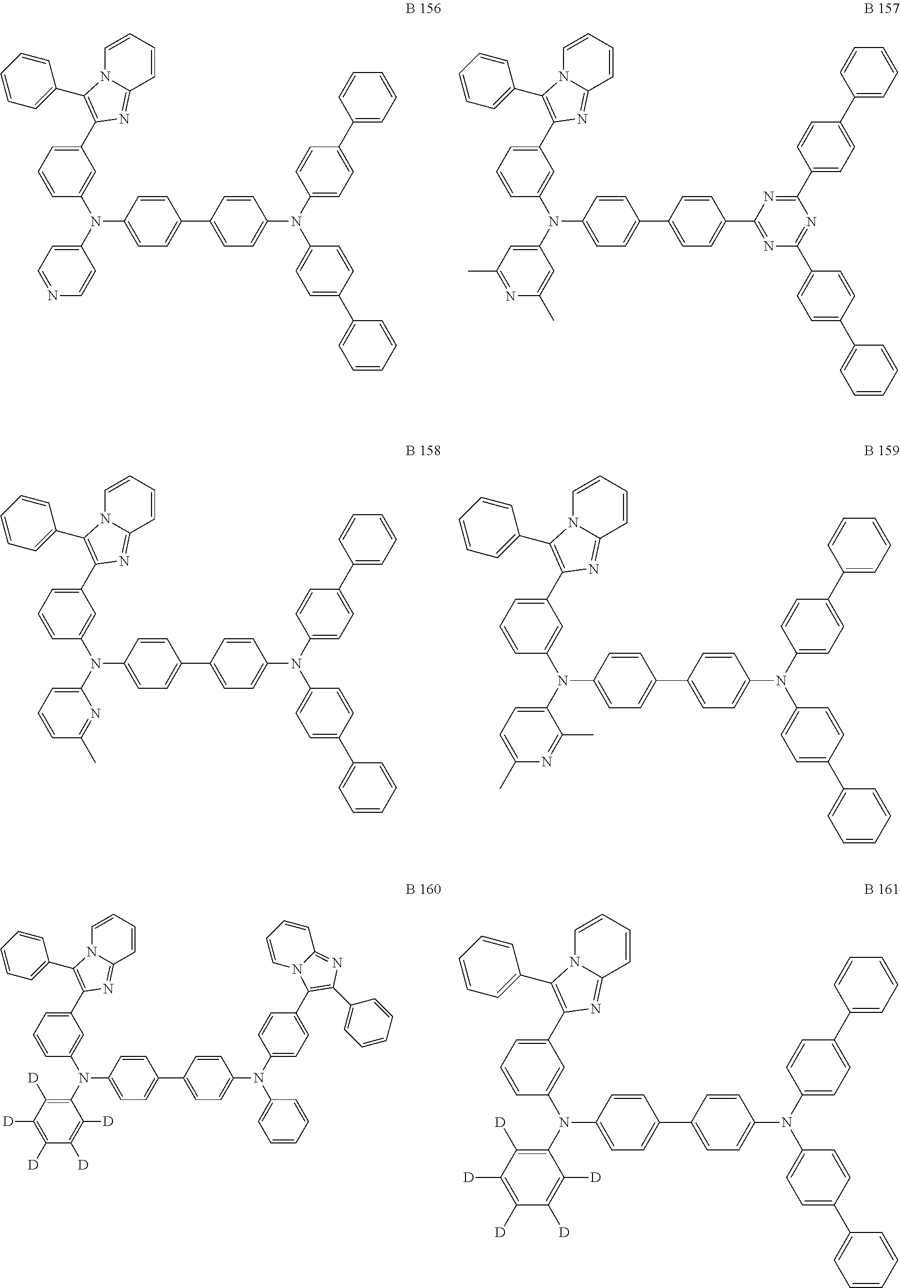

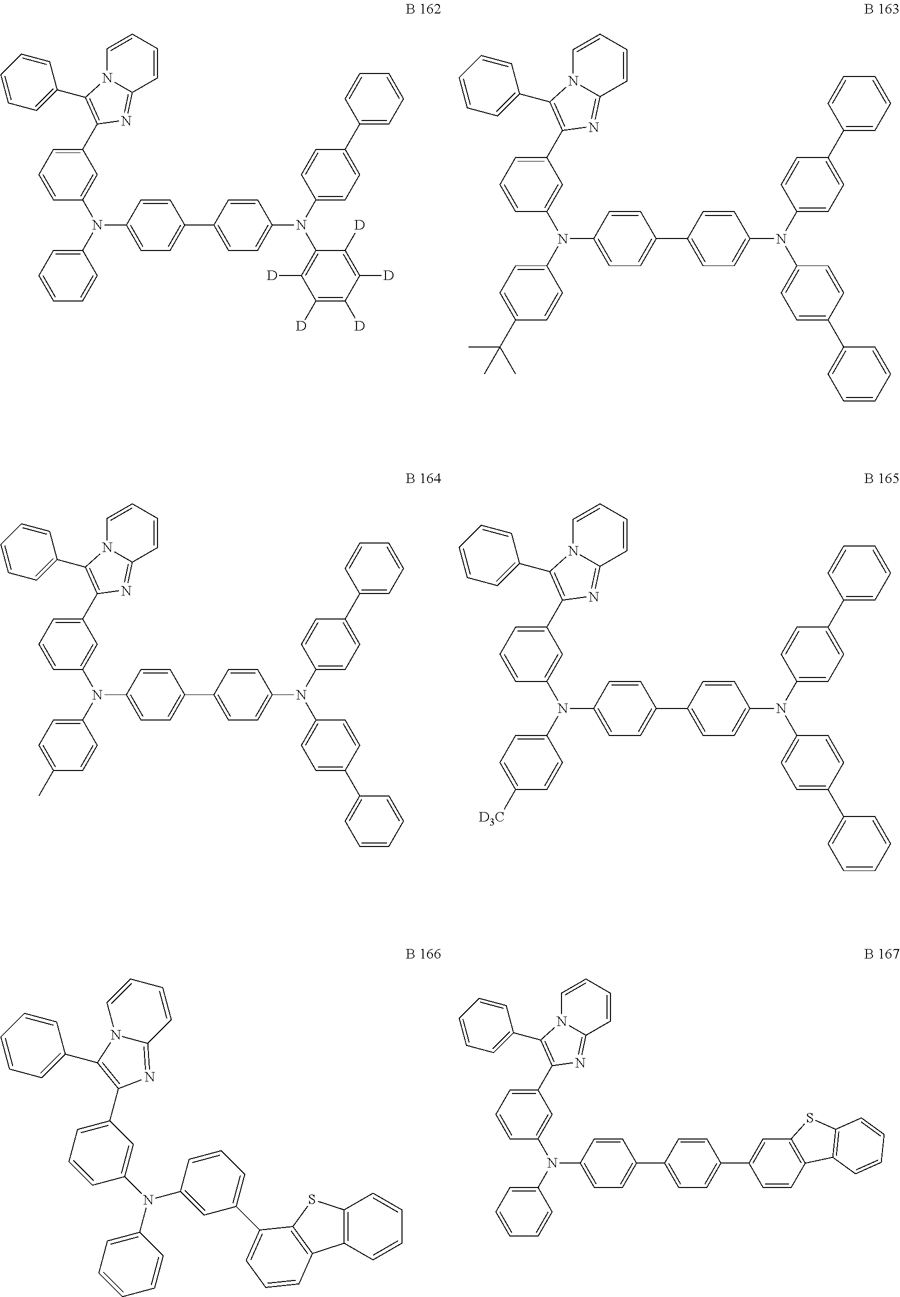

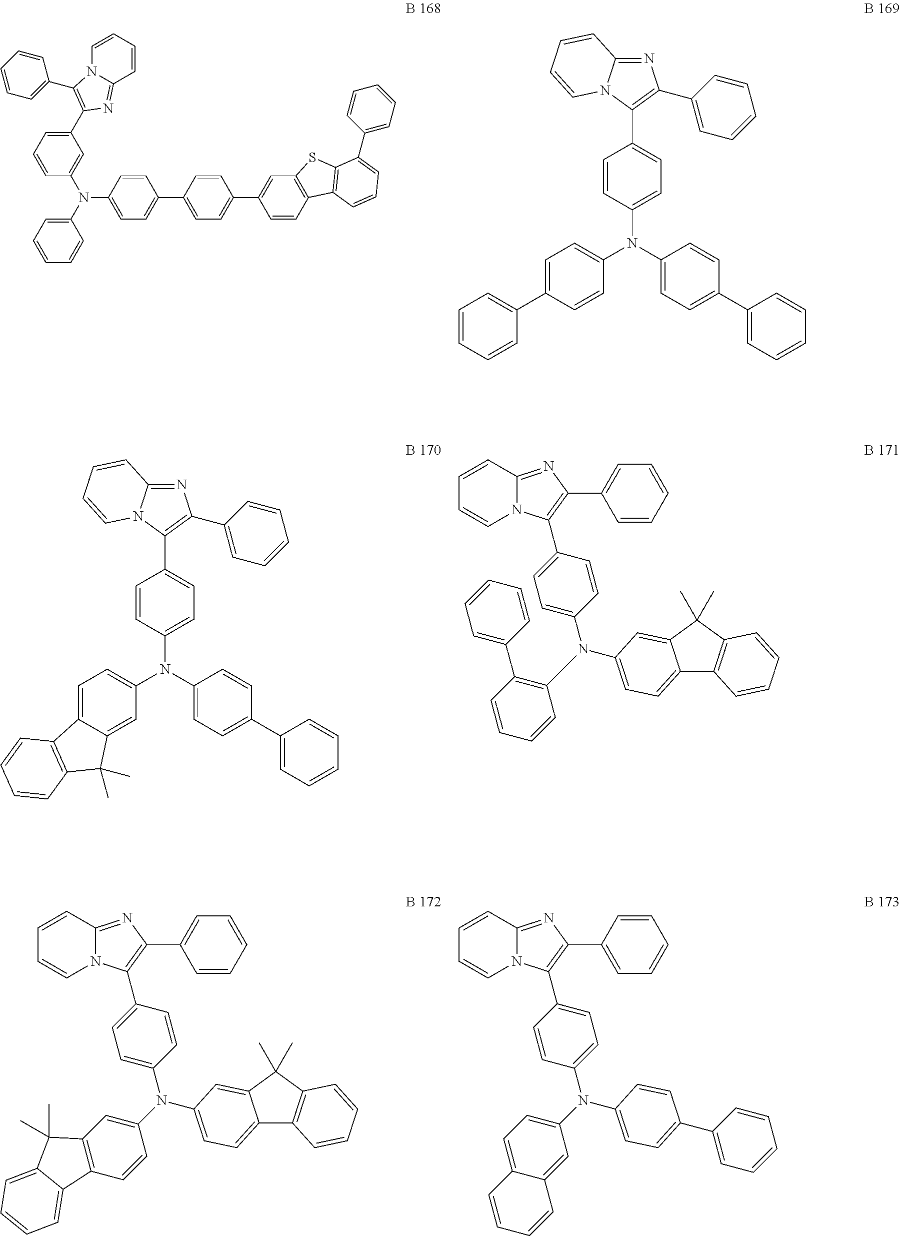

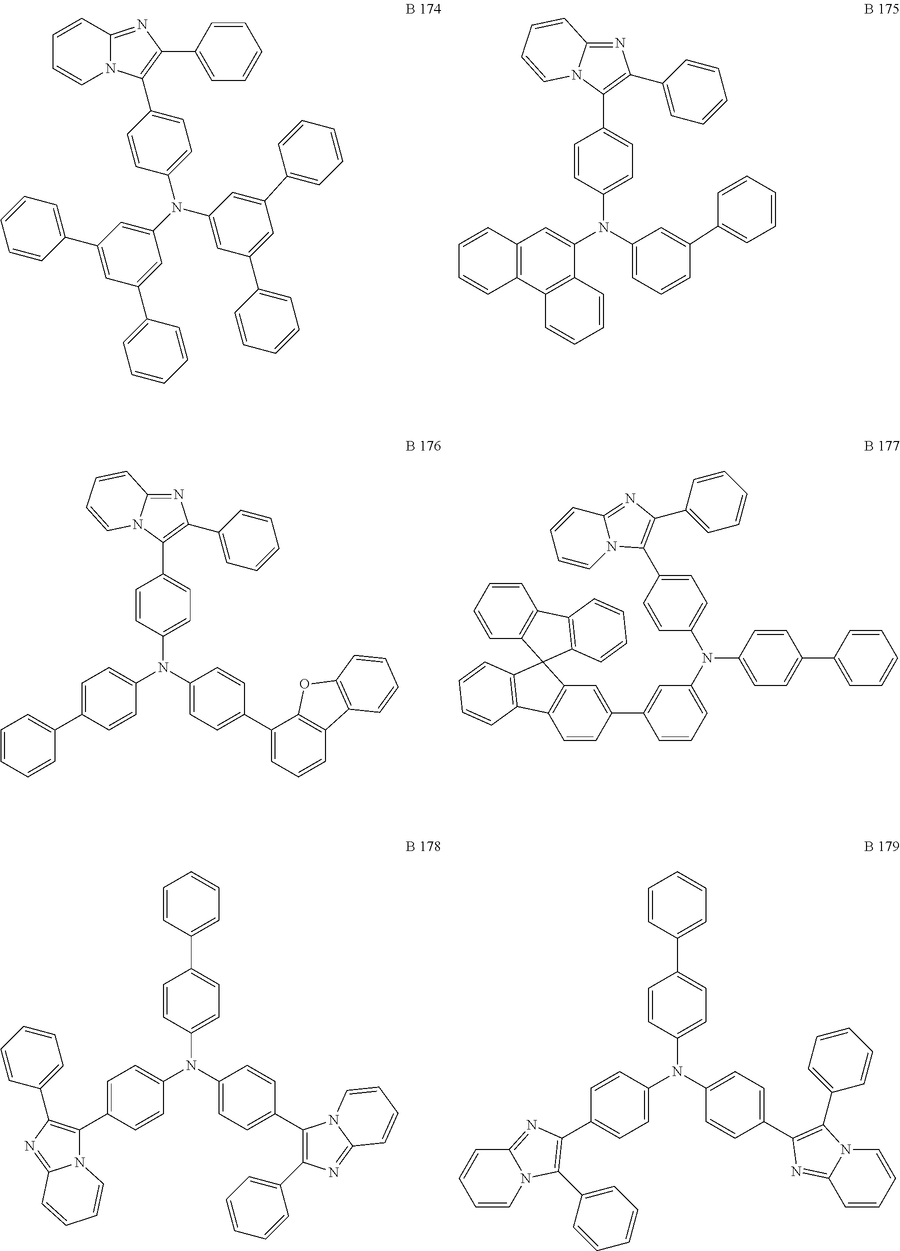

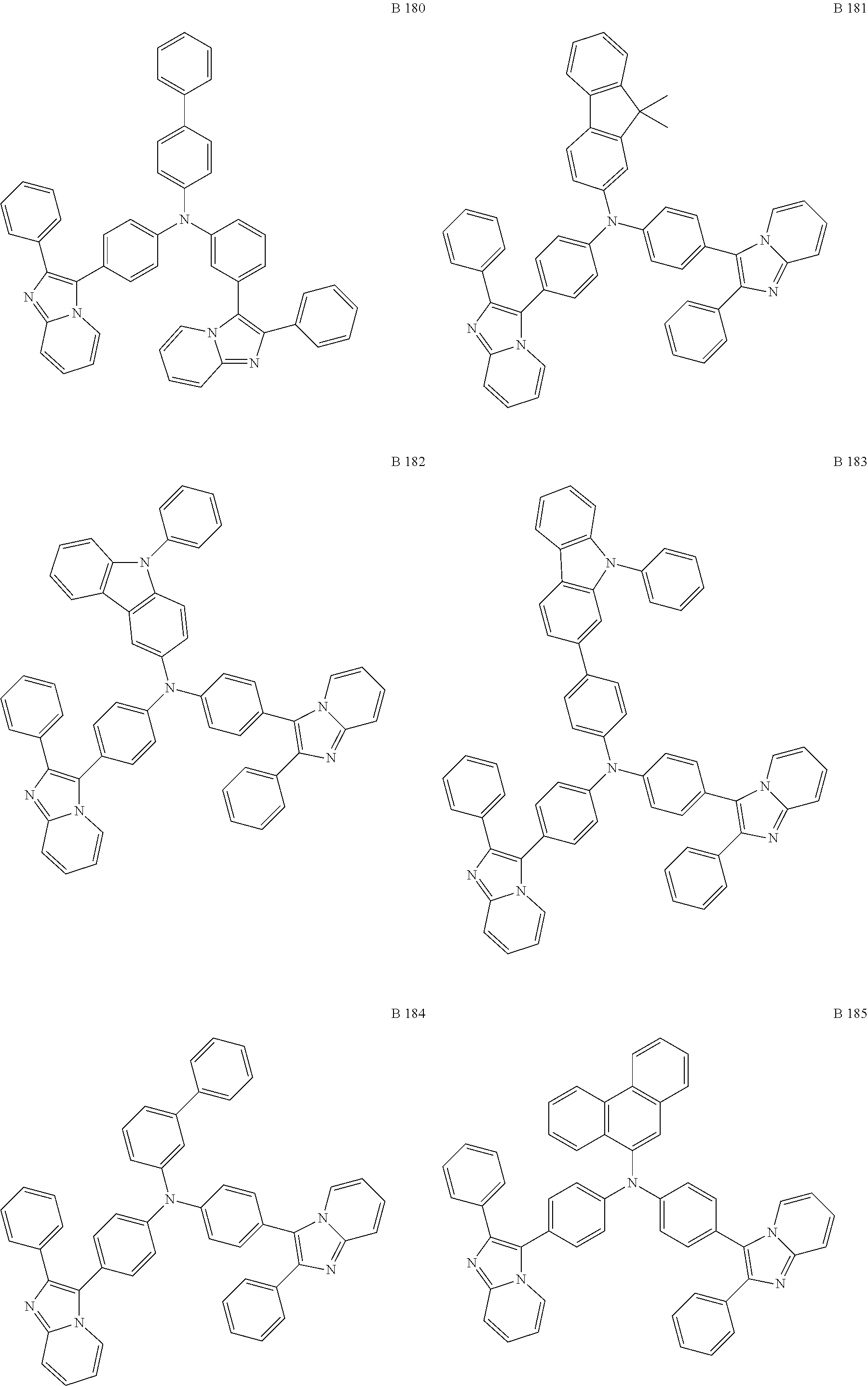

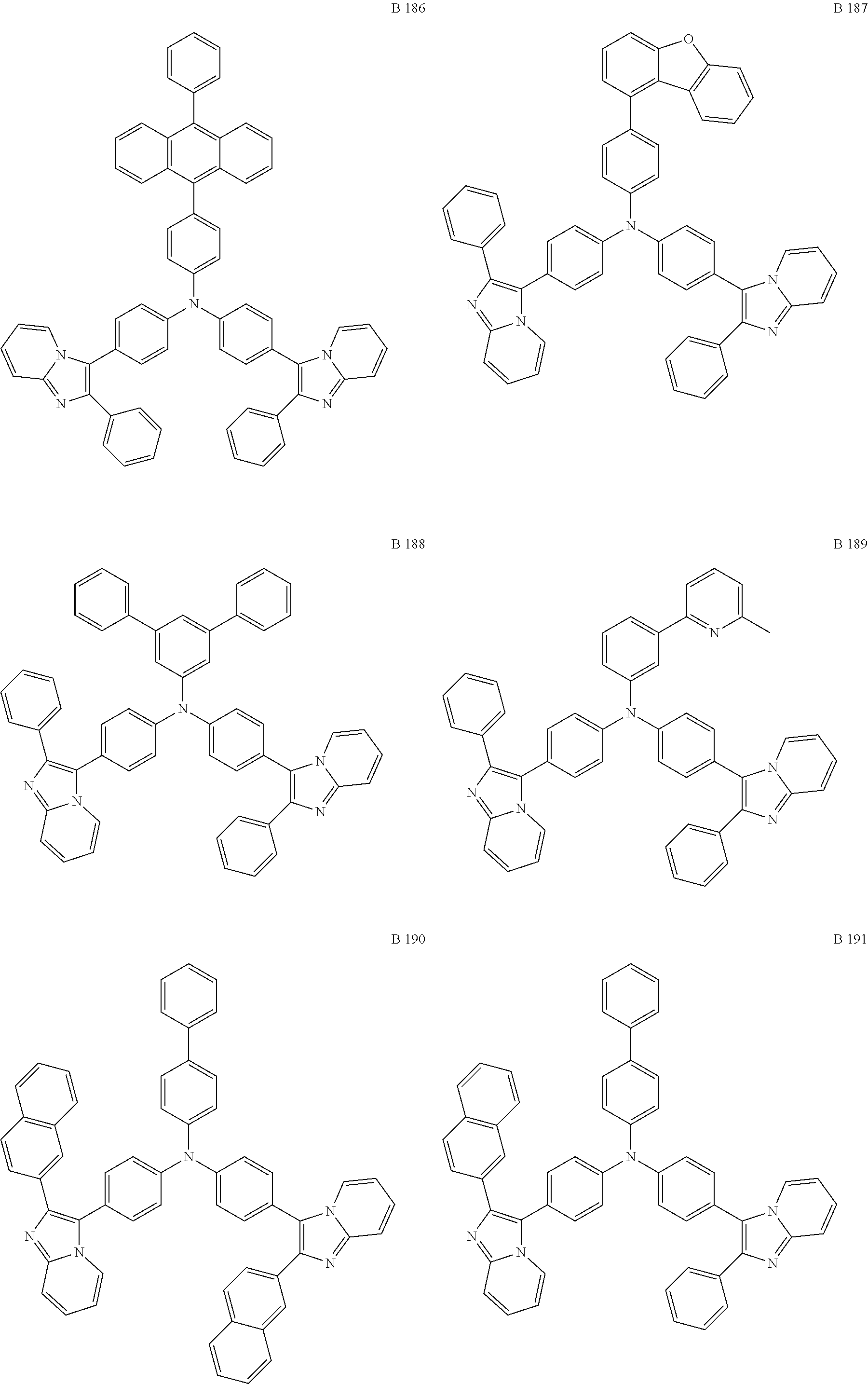

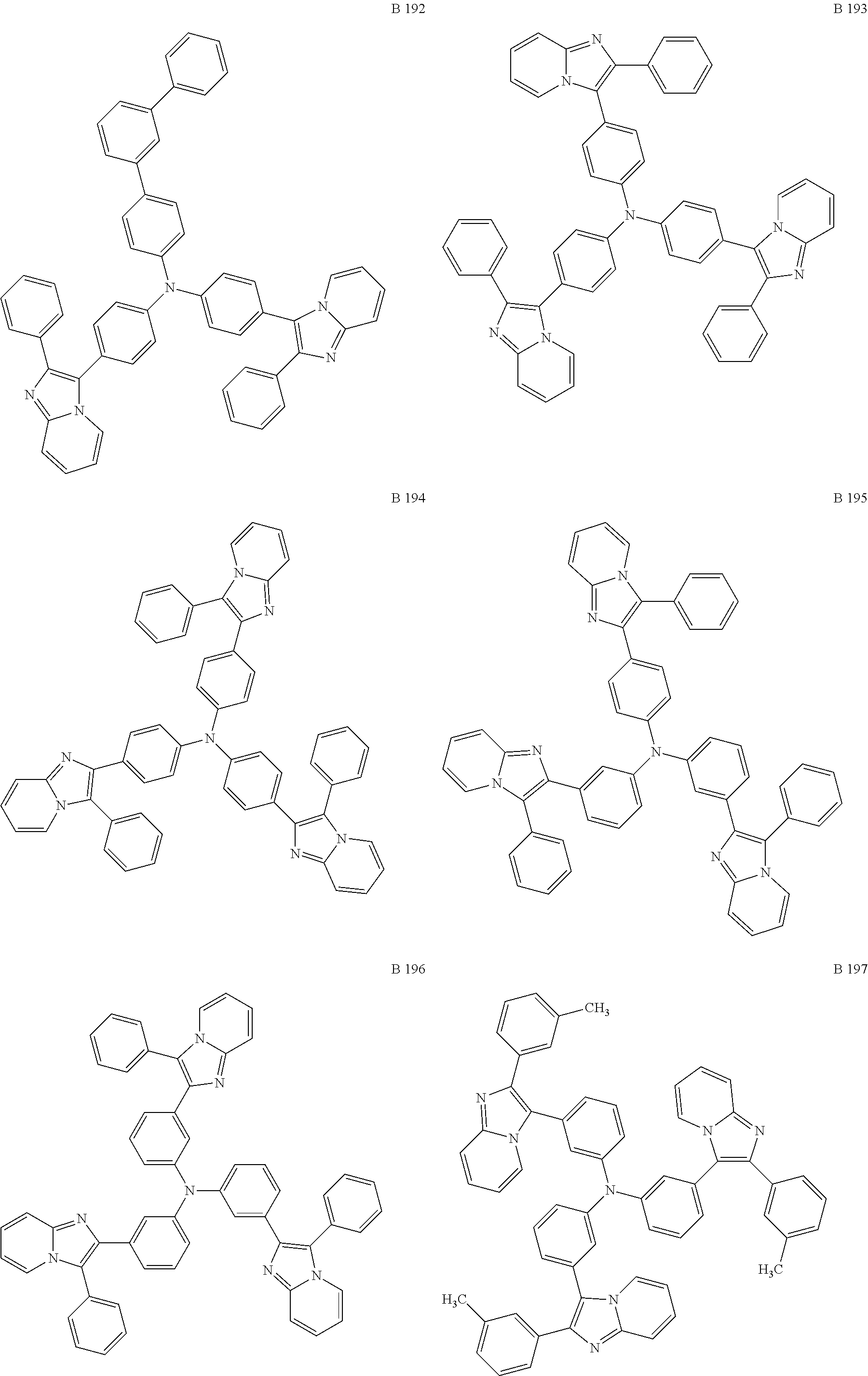

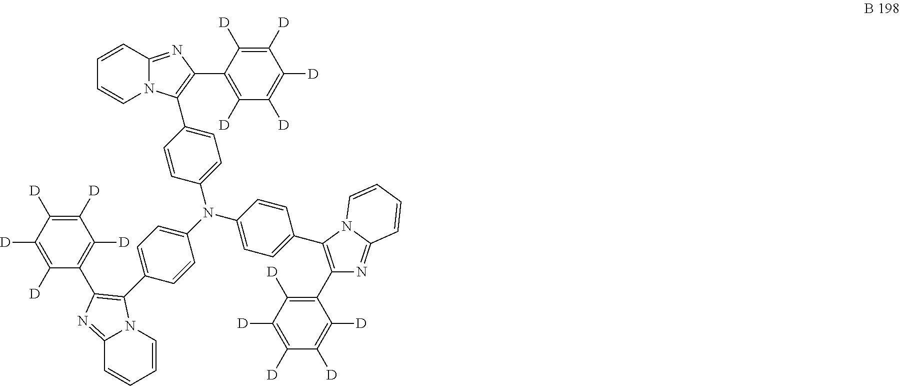 [SMILES: [2H]c1c([2H])c([2H])c(-c2nc3ccccn3c2-c2ccc(N(c3ccc(-c4c(-c5c([2H])c([2H])c([2H])c([2H])c5[2H])nc5ccccn45)cc3)c3ccc(-c4c(-c5c([2H])c([2H])c([2H])c([2H])c5[2H])nc5ccccn45)cc3)cc2)c([2H])c1[2H]]

10. An application of the compound of any one of claims 1-9 in an OLED device, wherein the compound of any one of claims 1-9 serves as a light extraction layer material of the OLED device.
